(19) United States
(12) Patent Application Publication
(10) Pub. No.: US 2022/0324997 A1
(43) Pub. Date: Oct. 13, 2022

(54) SSEA-4 BINDING MEMBERS

(71) Applicant: Scancell Limited, Oxford Oxfordshire (GB)

(72) Inventors: Linda Gillian Durrant, Nottingham Nottinghamshire (GB); Ji-aXin Chua, Nottingham Nottinghamshire (GB)

(21) Appl. No.: 17/640,245

(22) PCT Filed: Sep. 4, 2020
(86) PCT No.: PCT/EP2020/074878

§ 371 Date: Mar. 3, 2022

(30) Foreign Application Priority Data
Sep. 6, 2019 (GB) 1912882.6

(51) Int. Cl. C07K 16/30; C07K 16/44

(52) U.S. Cl. CPC C07K 16/3084; C07K 16/44; C07K 2317/24; C07K 2317/92; C07K 2317/732; C07K 2317/41; C07K 2317/734

(57) ABSTRACT

The disclosure relates to the expression of stage-specific embryonic antigen 4 (SSEA-4) on stem memory T-cells (TSCM), which can then be used as a target to isolate, activate and expand this T cell subset both in vivo and in vitro. It also relates to the pharmaceutical antibody composition binding SSEA-4 targeting TSCM, as well as methods for use thereof. The antibody of the disclosure recognises the SSEA-4 glycolipid and induces proliferation of TSCM which could be used to sort this unique population from blood for clinical expansion for adoptive T-cell transfer of T-cell receptor (TCR) transduced, chimeric antigen receptor (CAR)-T transduced or cells for haematopoietic stem cell transplant. Methods of use include, without limitation, in cancer therapies and diagnostics. Examples related to the antibody with the designation F2811.72.

### BACKGROUND

The present invention relates to the expression of stage-specific embryonic antigen 4 (SSEA-4) on stem memory T-cells (T_{SCM}), which can then be used as a target to isolate, activate and expand this T cell subset both in vivo and in vitro. It also relates to the pharmaceutical antibody composition binding SSEA-4 targeting T_{SCM}, as well as methods for use thereof. The antibody of the disclosure recognises the SSEA-4 glycolipid and induces proliferation of T_{SCM} which could be used to sort this unique population from blood for clinical expansion for adoptive T-cell transfer of T-cell receptor (TCR) transduced, chimeric antigen receptor (CAR)-T transduced or cells for haematopoietic stem cell transplant. Methods of use include, without limitation, in cancer therapies and diagnostics; as an agonist (IgG2) chimeric monoclonal antibody (mAb) for in vivo stimulation of T_{SCM} in either cancer or chronically virally infected patients or following chemotherapy.

SSEAs are globoseries glycolipids and are composed of 3 species: SSEA-1, SSEA-3 and SSEA-4 (Suzuki et al. 2013). Sialyl galactosyl globoside (sialyl Gb5Cer, SGG, MSGG) or SSEA-4 is a globo-series ganglioside synthesized from SSEA-3 by the enzyme ST3 beta-galactoside alpha-2,3-sialyltransferase 2 (ST3GAL2) (Saito et al. 2003). Due to the complexity in purifying and the number of genes involved in their synthesis, expression of these globosides has mainly been defined by mAbs. The main limitation of this approach is that most of these mAbs are of low specificity, making interpretation of individual globosides expression difficult to interpret. With this caveat the expression of SSEA-4 has been defined as: SSEA-4 is a component of glycosynapses of the plasma membrane. During human preimplantation development, SSEA-4 is first observed on the pluripotent cells of the inner cell mass and then is lost upon differentiation (Tondeur et al. 2008). After birth, human germ stem cells in the testis and ovary (Harichandan, Sivasubramaniyan, and Buhring 2013) as well as mesenchymal (Gang et al. 2007) and cardiac stem cells (Sandstedt et al. 2014) express SSEA-4 (Gang et al. 2007). It was identified via immunisation of animals with human embryonic carcinoma cells (human teratocarcinoma cells; tumours containing tissue derivatives of all three germ-layers) (Shevinsky et al. 1982; Kannagi et al. 1983; Wright and Andrews 2009) and is widely used as a cell surface marker to define human embryonic stem cells as well as their malignant counterparts, embryonic carcinoma cells (Kannagi et al. 1983; Lou et al. 2014; Henderson et al. 2002). In solid tumours, the overexpression of SSEA-4 has been found on glioblastoma (˜55% of grade I, ˜55% of grade II, ˜60% of grade III and ˜69% of grade IV astrocytoma) (Lou et al. 2014), renal cell carcinomas (Saito et al. 1997), breast cancer cells and breast cancer stem cells (Huang et al. 2013), basaloid lung cancer (Gottschling et al. 2013), epithelial ovarian carcinoma (Ye et al. 2010), and oral cancer (Noto et al. 2013). It is of great interest to identify ultra-specific glycan markers associated with and/or predictive of cancers, and develop antibodies against the markers for use in diagnosing and treating a broad spectrum of cancers. SSEA-4 is a glycan that is expressed on embryonic stem cells and is down regulated on adult stem cells. However, the inventors have unexpectedly shown its expression is retained on both human and mouse T_{SCM}. This is the first time a unique marker has been described on T_{SCM}.

Memory T-cells (including CD4⁺ and CD8⁺ memory T-cells) include several subsets: T_{SCM}, central memory (T_{CM}), transitional memory (T_{TM}) (described only in CD4⁺ memory T-cells), effector memory (T_{EM}), and terminal effector (T_{TE}) T-cells (Mateus et al. 2015; Takeshita et al. 2015). There is an on-going debate as to which methodology should be used to induce the generation of T_{SCM} cells from naïve, central memory or tumour infiltrating lymphocytes (TILs) to generate more potent anti-tumour cells for human clinical trials (Klebanoff, Gattinoni, and Restifo 2012).

Human T_{SCM} cells have been described as a long-lived memory T-cell population, sharing phenotypic similarities with naïve T-cells (CD45RO⁻, CCR7⁺, CD45RA⁺, CD62L⁺, CD27⁺, CD28⁺ and IL-7Rα⁺) whilst also highly expressing CD95, IL-2Rβ (CD122) and CXCR3 (Gattinoni et al. 2011). T_{SCM} cells are a clonally expanded primordial memory T subset which arises following antigenic stimulation and exhibit significantly enhanced proliferative and reconstitution capacities (Gattinoni et al. 2011).

Maintenance of long-lasting immunity is thought to depend on T_{SCM} cells, which constitute a small proportion, and are the least differentiated memory T-cell subset, approximately 2-4% of the total CD4⁺ and CD8⁺ T-cell population in the blood (Gattinoni et al. 2011; Lugli, Gattinoni, et al. 2013). T_{SCM} cells were first observed in a murine model of graft-versus-host disease (GVHD) by Zhang et al. (Zhang et al. 2005) who reported a new subset of post-mitotic CD44^{low}CD62^{high}CD8⁺ T-cells expressing Sca-1 (stem cell antigen 1), CD122 and Bcl-2. This population of T-cells was able to generate and sustain all allogeneic T-cell subsets in GVHD reactions. These alloreactive CD8⁺ T-cells were demonstrated to have enhanced self-renewal capacity and multipotency, and were capable of differentiating into T_{CM}, T_{EM}, and T_{TE} cells (Chahroudi, Silvestri, and Lichterfeld 2015; Zhang et al. 2005). In humans, an example came from the identification of a population of naïve yellow fever (YF)-specific CD8⁺ T-cells after vaccination, which were stably maintained for more than 25 years and were capable of ex vivo self-renewal (Fuertes Marraco et al. 2015). T_{SCM} cells can be identified by flow cytometry based on the simultaneous expression of several naïve markers together with the marker CD95 (Mahnke et al. 2013). There have been limited reports on antigen-specific T_{SCM} cells as the low frequency of these cells limits detailed characterisation. For example, <1% of total human T-cells are defined as CD8⁺CD45RA⁺CCR7⁺CD127⁺CD95⁺ viral-specific T_{SCM} cells. Human CMV-specific T_{SCM} cells can be detected at frequencies similar to those observed in other subsets, with a frequency of around ˜1/10,000 T-cells (Schmueck-Henneresse et al. 2015; Di Benedetto et al. 2015). Antigen-specific T_{SCM} cells have been shown to preferentially reside in the lymph nodes and less so in the spleen and bone marrow (Lugli, Dominguez, et al. 2013).

T_{SCM} cells may play a major role in specific anti-tumour response and long-term immune surveillance directed against tumours (Darlak et al. 2014; Coulie et al. 2014; Martin 2014). T_{SCM} cells with superior persistence capacity are also emerging as important players in the maintenance of long-lived T-cell memory and are thus considered an attractive population to be used in the adoptive transfer-based immunotherapy of cancer. However, the molecular signals regulating their generation remain poorly defined. Experiments conducted in the setting of adoptive immunotherapy revealed that T-cells deficient for two key transcription factors governing T cell differentiation, T-box transcription factor (T-bet) and Eomesodermin (eomes) were unable to trigger an anti-tumour response and expressed markers consistent with T_{SCM}. Therefore, the anti-tumour potential of T_{SCM} seems to rely more on their further differentiation into effector memory cells than in their intrinsic activity (Li et al. 2013).

Adoptive T-cell therapy is an effective strategy for cancer immunotherapy but the infused T-cells frequently become functionally exhausted and consequently offer a poor prognosis after transplantation into patients. Adoptive transfer of tumour antigen-specific T_{SCM} cells overcomes this shortcoming as T_{SCM} cells are close to naïve T-cells, but are also highly proliferative, long-lived, and produce a large number of effector T-cells in response to antigen stimulation. Adoptive cellular therapy using T-cells with tumour specificity derived from either natural TCRs or an artificial CAR has reached late phase clinical testing. Immunotherapeutic treatment of cancer using CAR-expressing T-cells is a relatively new approach in adoptive cell therapy. CAR-T cells have shown remarkable success in certain B cell malignancies, however, response rates against solid cancer shave been less successful to date. The strategy is based on genetically equipping T-cells with novel synthetic receptors that consist of an antibody-like recognition extracellular domain and a T-cell signalling intracellular domain. The direct identification of intact antigens that is provided by the antibody-derived binding domain of the receptor enables T-cells to bypass restrictions of the major histocompatibility complex (MHC)-mediated antigen recognition, so that a given CAR can be used in any patient regardless of its MHC haplotype. The MHC independence endows the CAR-T cells with a fundamental anti-tumour advantage, as some tumour cells downregulate the MHC expression to escape the TCR-mediated immune response (Garrido et al. 1993). However, the T-cells engineered to express the CAR of interest are still able to recognise and eradicate tumour cells. Moreover, by using CAR-T cells, the range of potential tumour targets can be broadened to epitopes that are beyond the scope of TCR-based recognition, e.g. it is possible to include not only proteins but also carbohydrates (Mezzanzanica et al. 1998) and glycolipids (Yvon et al. 2009) for tumour targeting.

The characteristics of T-cells selected for expansion and adoptive transfer are crucial in determining the persistence of transferred cells. Antigen-specific T-cells in the presence of infections or cancer can expand and differentiate into effector T-cells devoted to rapidly clearing pathogens, as well as memory T-cells that can persist long-term and defend against recurrence of disease. The memory T-cell compartment is heterogeneous and encompasses multiple subsets with distinctive properties. The immunological memory spectrum includes T_{SCM} cells which, like naïve T-cells express CD45RA, CCR7 and CD62L, but also CD95. While T_{SCM} cells can differentiate into central memory T-cells (T_{CM}) and effector memory T-cells (T_{EM} cells), and terminal effector T-cells (T_{TE}) they also have a marked potential for self-renewal as shown by serial transplantation experiments (Cieri et al. 2013). The contribution of different memory subsets to the maintenance of the overall memory compartment of antigen-specific T-cells has not been fully elucidated with the low frequency of T_{SCM} cells limiting their detailed characterisation (Schmueck-Henneresse et al. 2015). Strategies to generate, expand, and enable the redirection of T_{SCM} cells against cancer cells needs to be fully defined. Cieri and colleagues have described the generation of a large number of T_{SCM} cells, by priming naïve T-cells with anti-CD3/CD28 and low doses of IL-7 and IL-15 suggesting it is possible to generate, expand, and genetically engineer T_{SCM} cells in vitro from naïve precursors. However, the expanded cells no longer expressed CD45RA but expressed CD45RO so they could be T_{CM}. Further, the in vitro-generated T_{SCM} cells displayed enhanced proliferative capacity upon adoptive transfer into immunodeficient mice, a finding consistent with those naturally occurring T_{SCM} cells (Gattinoni and Restifo 2013; Cieri et al. 2013). Among the known memory T-cell populations, the T_{SCM} cell subset has profound implications for the design and development of effective vaccines as well as T-cell-based therapies (Restifo and Gattinoni 2013; Gattinoni et al. 2011; Lugli, Dominguez, et al. 2013). T_{SCM} cells may facilitate clinical development of cellular (CAR-T) immunotherapies (Han et al. 2013; Akinleye, Awaru, et al. 2013; Breton et al. 2014; Akinleye, Chen, et al. 2013; Novero et al. 2014; Suresh et al. 2014), however, the low number of T_{SCM} cells in circulating lymphocytes is limiting their application (Gattinoni and Restifo 2013).

Altered glycosylation is a feature of cancer cells, and several glycan structures are well-known tumour markers (Meezan et al. 1969; Hakomori 2002). These aberrant changes can include the overall increase in branching of N-linked glycans (Lau and Dennis 2008) and sialic acid content (van Beek, Smets, and Emmelot 1973), loss or overexpression of certain glycan epitopes (Sell 1990; Hakomori and Zhang 1997; Taylor-Papadimitriou and Epenetos 1994), persistence of truncated or emergence of novel glycans (Huang et al. 2013). Indeed, many tumours exhibit increased expression of certain glycolipids, especially the gangliosides, glycosphingolipids (GSLs) and sialic acid(s) attached to the glycan chain. Numerous studies have indicated that aberrant glycosylation is responsible for initial oncogenic transformation, as well as playing a key role in the induction of tumour invasion and metastasis (Hakomori 2002). Overexpression of a broad range of GSLs have been identified in various types of human malignancies: GD4 in melanoma (Nudelman et al. 1982), GD2 in neuroectodermal tumours (Cahan et al. 1982), fucosyl-GM1 in small cell lung carcinoma (Nilsson et al. 1986), Globo-H in breast and ovarian carcinomas (Chang et al. 2008), and stage-specific embryonic antigen (SSEA)-3 and SSEA-4 in breast and breast cancer stem cells (Chang et al. 2008).

Successful cancer immunotherapy is dependent on the generation of mAbs with good specificity and potent killing. The complexity of the glycome and altered expression of glycosyl transferases associated with malignant transformation make cancer cell-associated carbohydrates excellent targets (Christiansen et al. 2014; Dalziel et al. 2014; Daniotti et al. 2013; Hakomori 2002). Glycolipids are particularly attractive due to their dense cell surface distribution, mobility, and association with membrane microdomains, all of which contribute to their participation in a wide range of cellular signalling and adhesion processes (Fuster and Esko 2005; Hakomori 2002; Hakomori 2008).

Generating anti-glycolipid antibodies, however, is a challenging task as they do not provide T-cell help and the mAbs are usually low affinity IgMs.

### SUMMARY OF THE INVENTION

FG2811.72 (also abbreviated FG2811) mAb is a mouse IgG3 mAb, generated from mice immunised with glycol-engineered mouse fibroblast cell line, SSEA-3/-4-LMTK. Interestingly, FG2811 mAb recognised SSEA-4 specifically. SSEA-4 is similar to SSEA-3 in terms of structure, except that it has an additional terminal sialic acid residue. α-2,3-sialyltransferase, which is encoded by ST3GAL2 gene has been suggested to be the main enzyme contributes to the sialylation of SSEA-3 into SSEA-4. The FG2811 mAb binds specifically to SSEA-4 and does not cross react with SSEA-3. This is in contrast to the previously derived mAbs MC813, which the inventors found also bound SSEA-3 and Forssman, and MC613, which bound SSEA-3 and Globo-H. In contrast to MC813, FG2811 does not bind to red blood cells suggesting these cells do not express SSEA-4 and the binding of MC813 may be related to SSEA-3/Forssman expressions (Cooling and Hwang 2005). US2010/0047827 describes a mAb which binds to SSEA-4 but they also show it only binds the terminal disaccharide Neu5Ac(α2-3)Gal which can be expressed by a range of other globosides and also binds to SSEA-3 and Globo-H. US2016/0289340A1 does disclose some new anti-SSEA4 binding mAbs, which seem to be specific but in their assays MC813 is also SSEA-4 specific, in contrast to our results. Screening of binding of normal blood revealed that FG2811 but not MC813 recognised a small population of lymphocytes. This was further characterised as T_{SCM} In contrast the previous SSEA-4 mAb (MC813) which cross-reacts with SSEA-3 and Forssman antigens, this disclosure describes a highly SSEA-4 specific mAb, FG2811, which can stimulate the proliferation and maintenance of T_{SCM}.

In one aspect the present invention provides a specific binding member that binds specifically to SSEA-4 Neu5Ac(α2-3)Gal(β1-3)GalNAc(β1-3)Gal(α1-4)Gal(β1-4)Glc.

In a further aspect the present invention provides a method of identifying stem memory T-cells (T_{SCM}) by detecting the presence of SSEA-4 Neu5Ac(α2-3)Gal(β1-3)GalNAc(β1-3)Gal(α1-4)Gal(β1-4)Glc on the cell using a specific binding member of the invention.

In a further aspect the present invention provides a method of purifying stem memory T-cells (T_{SCM}) by detecting the presence of SSEA-4 Neu5Ac(α2-3)Gal(β1-3)GalNAc(β1-3)Gal(α1-4)Gal(β1-4)Glc on the cell using a specific binding member of the invention.

In a further aspect the invention provides a specific binding member capable of targeting stem memory T-cells (T_{SCM}). In a further aspect the invention provides a specific binding member capable of specifically binding to stem memory T-cells (T_{SCM}). In some aspects of the invention the specific binding member is capable of inducing proliferation of stem memory T-cells (T_{SCM}).

In a further aspect the present invention provides a specific binding member that binds to SSEA-4 Neu5Ac(α2-3)Gal(β1-3)GalNAc(β1-3)Gal(α1-4)Gal(β1-4)Glc wherein the isolated antibody or a binding fragment or member thereof is ultra-specific.

In a further aspect the present invention provides a specific binding member that binds to SSEA-4 Neu5Ac(α2-3)Gal(β1-3)GalNAc(β1-3)Gal(α1-4)Gal(β1-4)Glc wherein the specific binding member is capable of stimulating proliferation of stem memory T-cells (T_{SCM}).

In a further aspect the present invention provides a specific binding member that binds to SSEA-4 Neu5Ac(α2-3)Gal(β1-3)GalNAc(β1-3)Gal(α1-4)Gal(β1-4)Glc wherein the specific binding member is capable of activating stem memory T-cells (T_{SCM}).

In some aspects of the invention, the specific binding member of the invention is capable of stimulating proliferation of stem memory T-cells (T_{SCM}) by at least about 10%, at least about 20%, at least about 30%, at least about 40%, at least about 50%, at least about 60%, at least about 70%, at least about 80%, at least about 90% or at least about 100% compared to stem memory T-cells (T_{SCM}) in the absence of the specific binding member.

In some aspects of the invention, the activation of stem memory T-cells (T_{SCM}) can be measured by the production of a specific marker or by an increased functional effect of the cells. In some aspects of the invention, the specific binding member of the invention is capable of activating stem memory T-cells (T_{SCM}) by at least about 10%, at least about 20%, at least about 30%, at least about 40%, at least about 50%, at least about 60%, at least about 70%, at least about 80%, at least about 90% or at least about 100% compared to stem memory T-cells (T_{SCM}) in the absence of the specific binding member.

In some aspects of the invention the specific binding member may be capable of binding to glycolipid-presented Neu5Ac(α2-3)Gal(β1-3)GalNAc(β1-3)Gal(α1-4)Gal(β1-4)Glc with an affinity (K_{d}) of less than about 10⁻⁸M. The specific binding member may be capable of binding glycolipid-presented with an affinity (K_{d}) of about 10⁻⁹M. The specific binding member may be capable of binding glycolipid-presented with an affinity (K_{d}) of less than about 10⁻⁸M, 10⁻⁹M, 10⁻¹⁰M, 10⁻¹¹M or 10⁻¹²M.

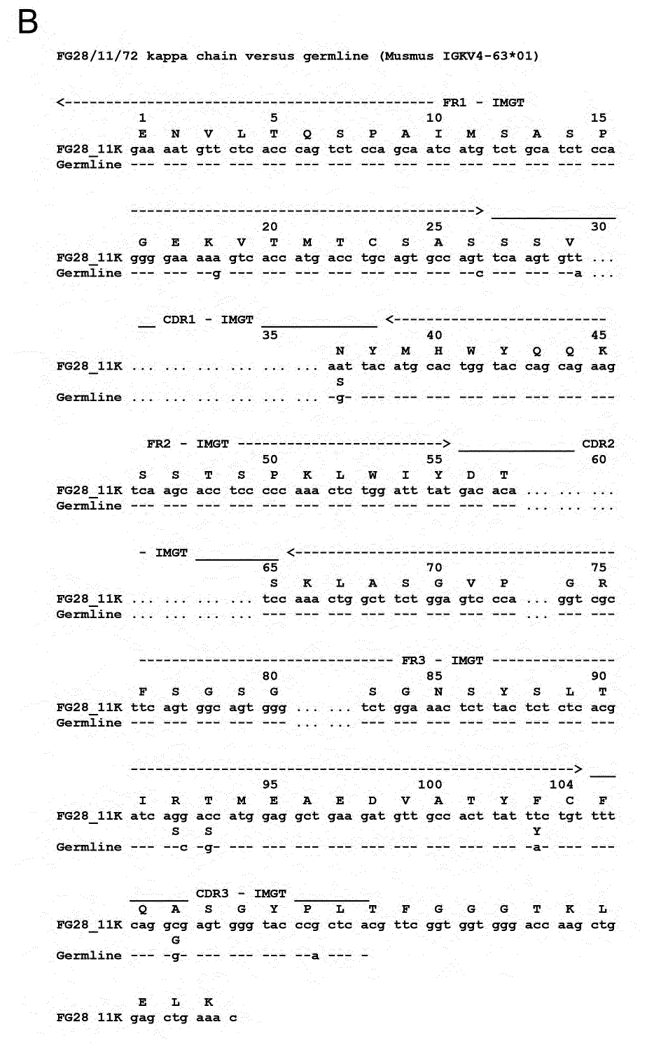

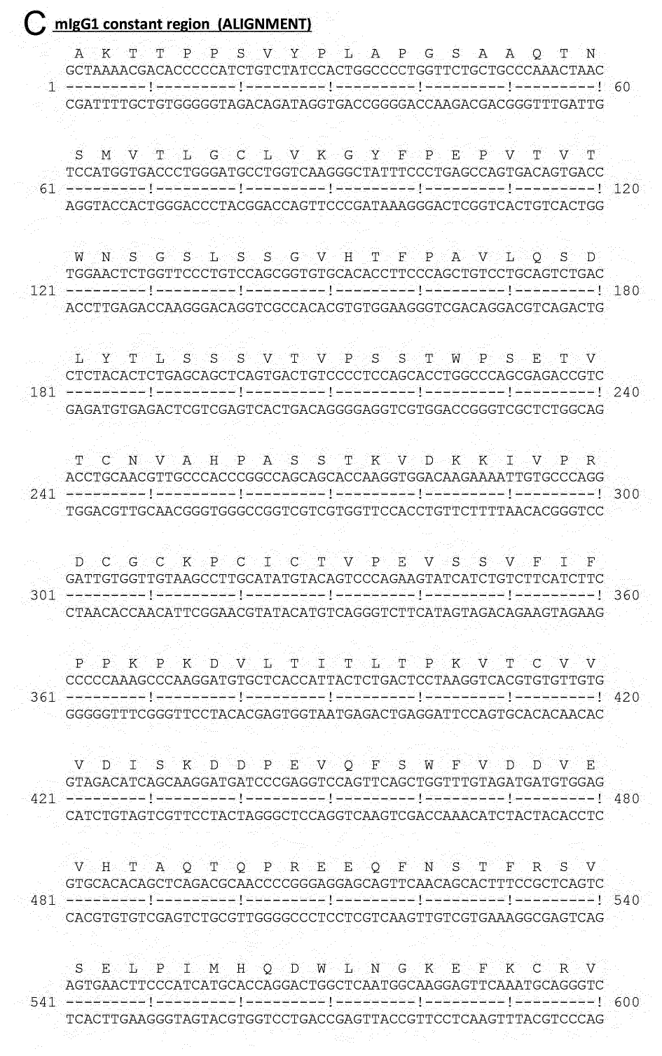

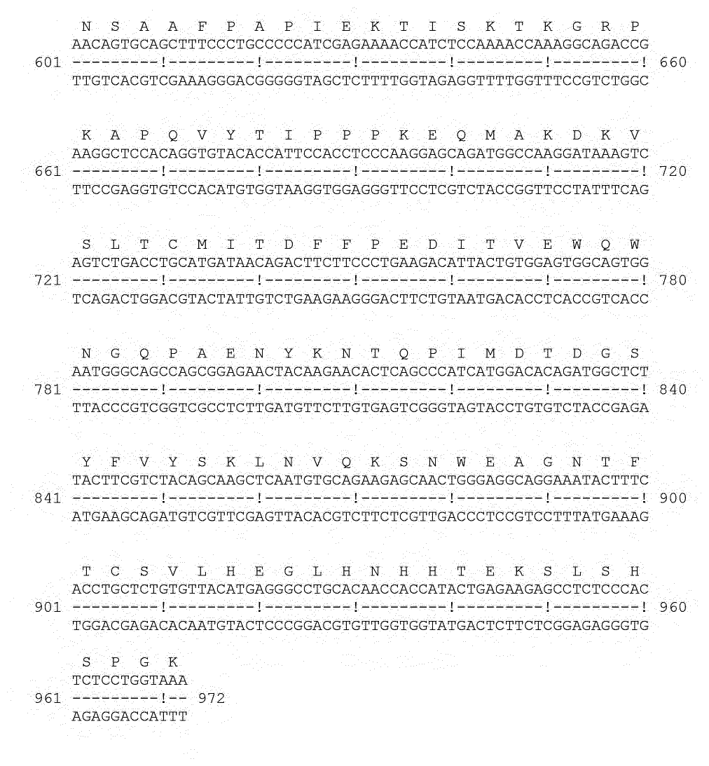

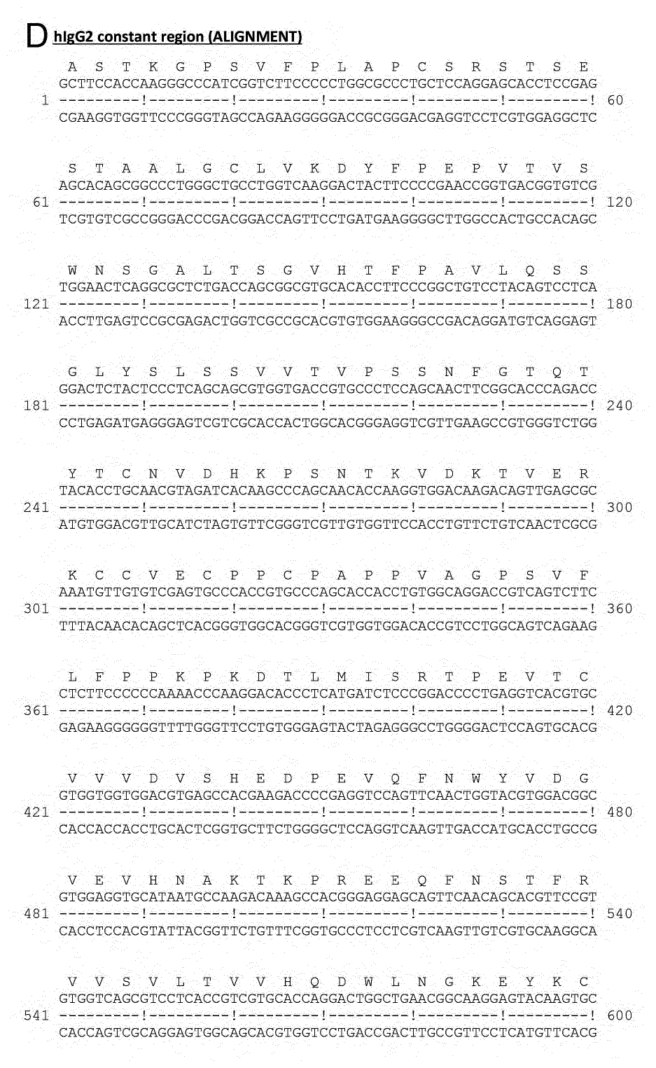

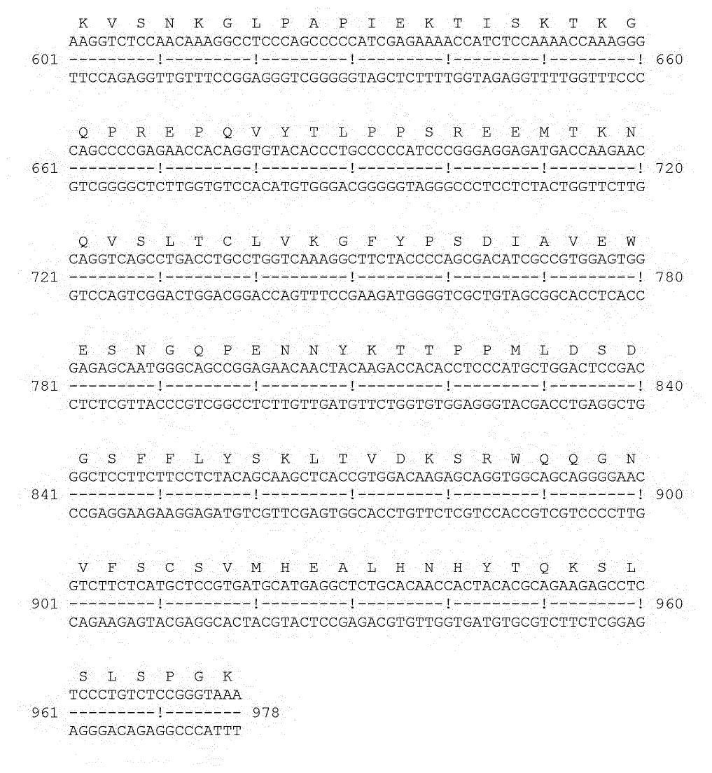

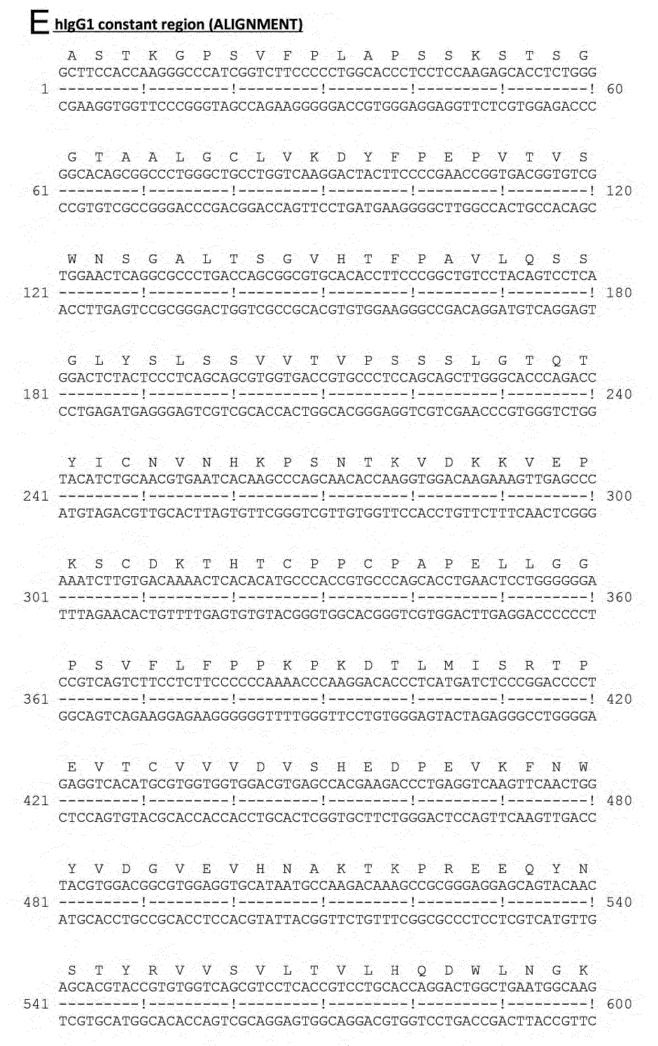

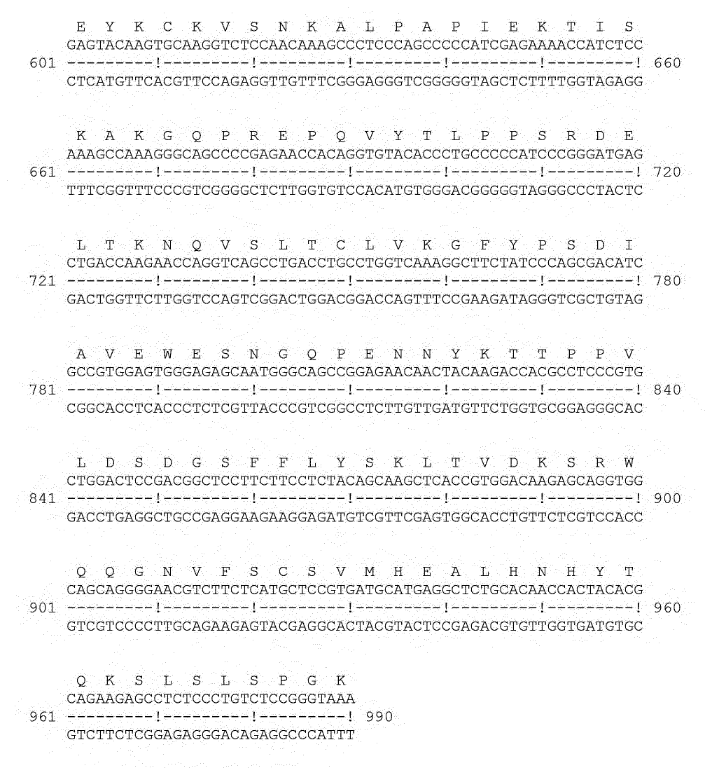

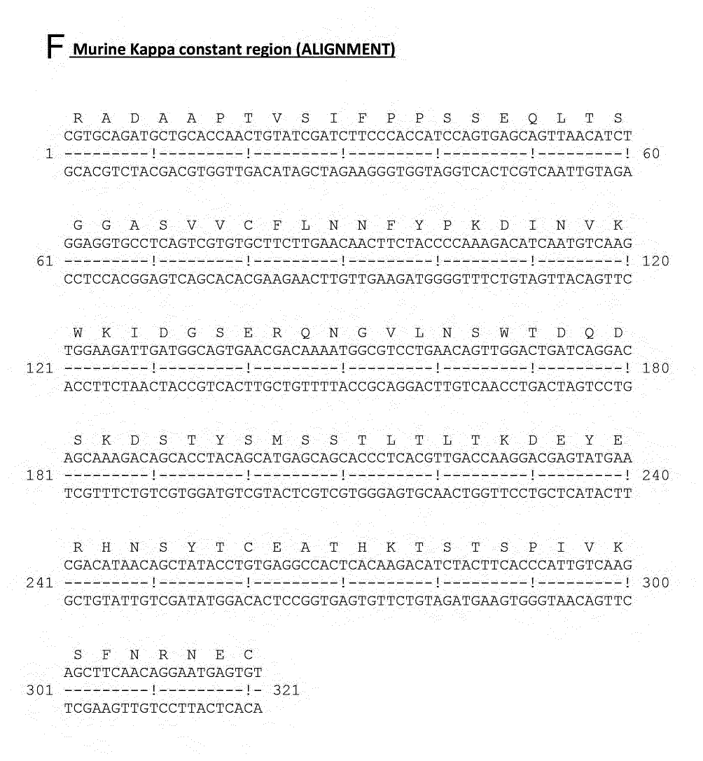

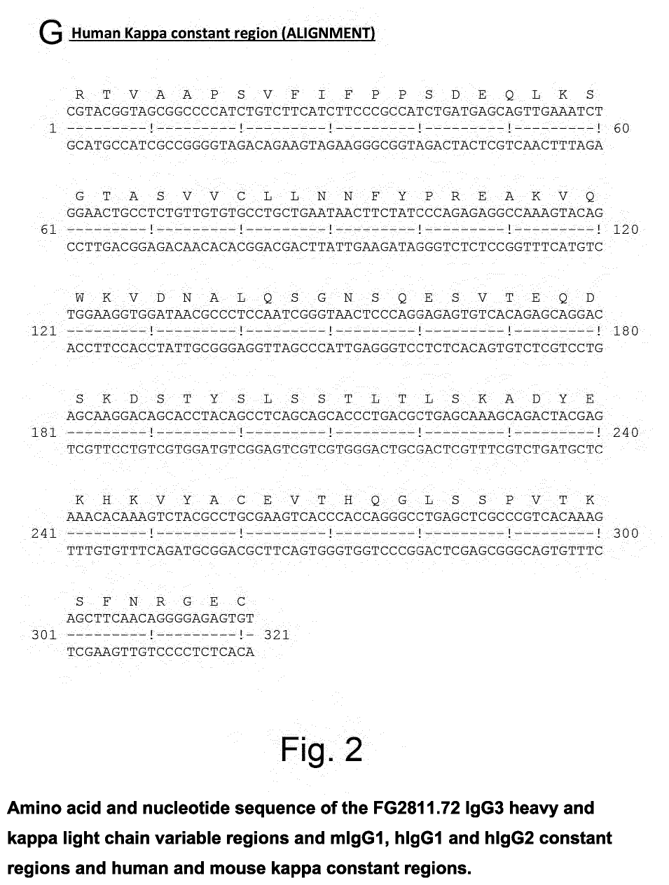

A further aspect of the invention provides a specific binding member comprising heavy chain binding domains CDR1, CDR2 and CDR3, and light chain binding domains CDR1, CDR2 and CDR3. The invention may provide a specific binding member comprising one or more binding domains selected from the amino acid sequence of residues 27 to 38 (CDRH1), 56-65 (CDRH2) and 105 to 113 (CDRH3) of FIGS. 2a and 2b.

The specific binding member of the invention may comprise an amino acid sequence substantially as set out as 1 to 126 (VH) of FIG. 2a. In one embodiment of the invention, the specific binding member of the invention comprises a binding domain, which comprises an amino acid sequence substantially as set out as residues 105 to 113 (CDRH3) of the amino acid sequence FIG. 2a. In this embodiment of the invention, the specific binding member may additionally comprise one or both, preferably both, of the binding domains substantially as set out as residues 27 to 38 (CDRH1) and residues 56 to 65 (CDRH2) of the amino acid sequence shown in FIG. 2a.

In another aspect, the present invention provides a specific binding member comprising one or more binding domains selected from the amino acid sequence of residues 27 to 38 (CDRL1), 56-65 (CDRL2) and 105 to 113 (CDRL3) of FIG. 2b.

In one aspect of the invention, the binding domain may comprise an amino acid sequence substantially as set out as residues 105 to 113 (CDRL3) of the amino acid sequence of FIG. 2b. In this embodiment, the specific binding member may additionally comprise one or both, preferably both, of the binding domains substantially as set out as residues 27 to 38 (CDRL1) and residues 56 to 65 (CDRL2) of the amino acid sequence shown in FIG. 2b.

In some embodiments of the invention, the variable heavy and/or light chain may comprise HCDR1-3 and LCDR1-3 of antibody FG2811. In some embodiments of the invention the variable heavy and/or light chain may comprise HCDR1-3 and LCDR1-3 of antibody FG2811, and framework regions of FG2811.

Specific binding members which comprise a plurality of binding domains of the same or different sequence, or combinations thereof, are included within the present invention. Each binding domain may be carried by a human antibody framework. For example, one or more framework regions may be substituted for the framework regions of a whole human antibody or of the variable region thereof.

One isolated specific binding member of the invention comprises the sequence substantially as set out as residues 1 to 123 (VL) of the amino acid sequence shown in FIG. 2b.

In some embodiments specific binding members having sequences of the CDRs of FIG. 2a may be combined with specific binding members having sequences of the CDRs of FIG. 2b.

In one embodiment, the specific binding member may comprise a light chain variable sequence comprising one or more (i.e. 1, 2 or 3) of LCDR1, LCDR2 and LCDR3, wherein:

LCDR1 comprises SSVNY

LCDR2 comprises DTS, and

LCDR3 comprises FQASGYPLT; and

a heavy chain variable sequence comprising one or more (i.e. 1, 2 or 3) of HCDR1, HCDR2 and HCDR3, wherein

HCDR1 comprises GFSLNSYG

HCDR2 comprises IWGDGST, and

HCDR3 comprises TKPGSGYAF.

In a further aspect, the invention provides a specific binding member comprising a VH domain comprising residues 1 to 126 of the amino acid sequence of FIG. 2a, and a VL domain comprising residues 1 to 123 of the amino acid sequence of FIG. 2b.

In certain embodiments, the specific binding member is a human antibody, chimeric antibody, or humanised antibody. In some aspects of the invention, the specific binding member is a monoclonal antibody. In some aspects of the invention, the specific binding member is a polyclonal antibody.

The invention also encompasses specific binding members as described above, but in which the sequence of the binding domains are substantially as set out in FIG. 2. Thus, specific binding members as described above are provided, but in which in one or more binding domains differ from those depicted in FIG. 2 by, from 1 to 5, from 1 to 4, from 1 to 3, 2 or 1 amino acid substitution(s).

The invention also encompasses specific binding members having the capability of binding to the same epitopes as the VH and VL sequences depicted in FIG. 2. The epitope of an isolated antibody or a binding fragment or member thereof is the region of its antigen to which the isolated antibody or a binding fragment or member thereof binds. Two antibodies or a binding fragments or members thereof bind to the same or overlapping epitope if each competitively inhibits (blocks) binding of the other to the antigen. That is, a 1×, 5×, 10×, 20× or 100× excess of one isolated antibody or a binding fragment or member thereof inhibits binding of the other by at least 50% but preferably by at least 75%, 90% or even 99% as measured in a competitive binding assay compared to a control lacking the competing antibody (see, e.g., (Junghans et al. 1990) which is incorporated herein by reference).

In a preferred embodiment of the invention the competing specific binding member competes for binding to SSEA-4, Neu5Ac(α2-3)Gal(β1-3)GalNAc(β1-3)Gal(α1-4)Gal(β1-4)Glc only attached to a glycolipid with an antibody comprising a VH chain having the amino acid sequence of residues 1 to 126 of FIG. 2a and a VL chain having the amino acid sequence of residues 1 to 123 of FIG. 2b.

Preferably, competing specific binding member are antibodies, for example mAbs, or any of the antibody fragments mentioned throughout this document.

Once a single, archetypal mAb, for example an FG2811 mAb, has been isolated that has the desired properties described herein, it is straightforward to generate other mAbs with similar properties, by using art-known methods. For example, the method of e.g., (Jespers et al. 1994), which is incorporated herein by reference, may be used to guide the selection of mAbs having the same epitope and therefore similar properties to the archetypal mAb. Using phage display, first the heavy chain of the archetypal antibody is paired with a repertoire of (preferably human) light chains to select a glycan-binding mAb, and then the new light chain is paired with a repertoire of (preferably human) heavy chains to select a (preferably human) glycan-binding mAb having the same epitope as the archetypal mAb.

MAbs that are capable of binding SSEA-4 only attached to a glycolipid and induce ADCC and/or CDC and are at least 90%, 95% or 99% identical in the VH and/or VL domain to the VH or VL domains of FIG. 2, are included in the invention. Reference to the 90%, 95%, or 99% identity may be to only the framework regions of the VH and/or VL domains. In particular, the CDR regions may be identical, but the framework regions may vary by up to 1%, 5%, or 10%. Preferably such antibodies differ from the sequences of FIG. 2 by a small number of functionally inconsequential amino acid substitutions (e.g., conservative substitutions), deletions, or insertions. In any embodiment of the invention, the specific binding pair may be an antibody or an antibody fragment, Fab, (Fab′)2, scFv, Fv, dAb, Fd or a diabody. In some embodiments the antibody is a polyclonal antibody. In other embodiments the antibody is a monoclonal antibody. Antibodies of the invention may be humanised, chimeric or veneered antibodies, or may be non-human antibodies of any species. In one embodiment the specific binding partner of the invention is mouse antibody FG2811 which comprises a heavy chain as depicted in FIG. 2a and a light chain as depicted in FIG. 2b.

Specific binding members of the invention may carry a detectable or functional label.

In further aspects, the invention provides an isolated nucleic acid encoding a specific binding member of the invention, and methods of preparing specific binding members of the invention which comprise expressing said nucleic acids under conditions to bring about expression of said binding member, and recovering the binding member. Isolated nucleic acids encoding specific binding members that are capable of binding specifically to SSEA-4 and are at least 90%, at least 95% or at least 99% identical to the sequences provided herein are included in the invention.

Specific binding members of the invention may be used in a method of treatment or diagnosis of the human or animal body, such as a method of treatment of a tumour in a patient (preferably human) which comprises administering to said patient an effective amount of a specific binding member of the invention. The invention also provides a specific binding member of the present invention for use in medicine, preferably for use in treating a tumour, as well as the use of a specific binding member of the present invention in the manufacture of a medicament for the diagnosis or treatment of a tumour. The tumour may be a gastric, colorectal, pancreatic, lung, ovarian or breast tumour.

Disclosed herein is the antigen to which the specific binding members of the present invention bind. A SSEA-4 which is capable of being bound, preferably specifically, by a specific binding member of the present invention may be provided. The SSEA-4 may be provided in isolated form, and may be used in a screen to develop further specific binding members therefor. For example, a library of compounds may be screened for members of the library which bind specifically to the SSEA-4.

In a further aspect the invention provides an isolated specific binding member capable of binding SSEA-4 containing glycans, preferably of the first aspect of the invention (i.e. Neu5Ac(α2 3)Gal(β1 3)GalNAc(β1 3)Gal(α1 4)Gal(β1 4)Glc), for use in the diagnosis or prognosis of gastric, colorectal, pancreatic, lung, ovarian and breast tumours.

In a further aspect of the invention there is provided a method of inducing proliferation of stem memory T-cells (T_{SCM}) ex vivo comprising contacting the stem memory T-cells (T_{SCM}) with a specific binding member of the invention.

In a further aspect of the invention there is provided a cell culture medium for inducing proliferation of stem memory T-cells (T_{SCM}) comprising a specific binding member of the invention.

In a further aspect of the invention there is provided a method of inducing proliferation of stem memory T-cells (T_{SCM}) in vivo comprising administering a subject with a specific binding member of the invention.

In a further aspect of the invention there is provided a binding member of the invention for use in therapy. In a further aspect of the invention there is provided a method of treating a patient wherein the method comprises administering a specific binding member of the invention to the patient in need thereof.

In a further aspect of the invention there is provided a specific binding member of the invention for use in a method of treating an autoimmune disease, HIV, adult T-cell leukaemia or graft versus host disease.

In a further aspect of the invention there is provided a method of treating or preventing cancer, comprising administering a specific binding member of the invention to a subject in need of thereof. In a further aspect of the invention there is provided a method of treating or preventing chronically virally infected patients, comprising administering a specific binding member of the invention to a subject in need of thereof.

In a further aspect of the invention there is provided a method of treating or preventing an autoimmune disease, HIV, adult T-cell leukaemia or graft versus host disease, comprising administering a specific binding member of the invention to a subject in need of thereof.

In a further aspect of the invention there is provided a cell culture comprising stem memory T-cells (T_{SCM}) and a specific binding member of the invention wherein the proliferation rate is enhanced by at least about 10% when compared to a corresponding cell culture comprising stem memory T-cells (T_{SCM}) without a specific binding member of the invention.

In some aspects of the invention the proliferation rate of a cell culture comprising stem memory T-cells (T_{SCM}) and a specific binding member of the invention is enhanced by at least about 20%, at least about 30%, at least about 40%, at least about 50%, at least about 60%, at least about 70%, at least about 80%, at least about 90% or at least about 100% when compared to a corresponding cell culture comprising stem memory T-cells (T_{SCM}) without a specific binding member of the invention.

In a further aspect of the invention there is provided a method of purifying stem memory T-cells (T_{SCM}) using a specific binding member of the invention wherein the proportion of stem memory T-cells (T_{SCM}) in the cell population is enhanced by at least about 10% when compared to a corresponding cell population that has not been purified using a specific binding member of the invention.

In some aspects of the invention the proportion of stem memory T-cells (T_{SCM}) in the purified cell population is enhanced by at least about 20%, at least about 30%, at least about 40%, at least about 50%, at least about 60%, at least about 70%, at least about 80%, at least about 90% or at least about 100% when compared to a corresponding cell population that has not been purified using a specific binding member of the invention.

In some aspects of the invention, the specific binding member of the invention is an isolated antibody or a binding fragment or member thereof.

The invention further provides a method for diagnosis of cancer comprising using a specific binding member of the invention to detect SSEA4 containing glycans in a sample from an individual. In some diagnostic methods of the invention, the pattern of glycans detected by the binding member may be used to stratify therapy options for the individual.

These and other aspects of the invention are described in further detail below.

As used herein, a “specific binding member” is a member of a pair of molecules, which have binding specificity for one another. The members of a specific binding pair may be naturally derived or wholly or partially synthetically produced. One member of the pair of molecules has an area on its surface, which may be a protrusion or a cavity, which specifically binds to and is therefore complementary to a particular spatial and polar organisation of the other member of the pair of molecules. Thus, the members of the pair have the property of binding specifically to each other. Examples of types of specific binding pairs are antigen-antibody, biotin-avidin, hormone-hormone receptor,5 receptor-ligand, enzyme-substrate. The present invention is generally concerned with antigen-antibody type reactions, although it also concerns small molecules, which bind to the antigen defined herein.

As used herein, “treatment” includes any regime that can benefit a human or non-human animal, preferably mammal. The treatment may be in respect of an existing condition or may be prophylactic (preventative treatment).

As used herein, a “tumour” is an abnormal growth of tissue. It may be localised (benign) or invade nearby tissues (malignant) or distant tissues (metastatic). Tumours include neoplastic growths, which cause cancer and include oesophageal, colorectal, gastric, breast, ovarian and endometrial tumours, as well as cancerous tissues or cell lines including, but not limited to, leukaemic cells. As used herein, “tumour” also includes within its scope endometriosis.

The term “antibody” as used herein refers to immunoglobulin molecules and immunologically active portions of immunoglobulin molecules, i.e., molecules that contain an antigen binding site that specifically binds an antigen, whether natural or partly or wholly synthetically produced. The term also covers any polypeptide or protein having a binding domain, which is or is homologous to, an antibody binding domain. These can be derived from natural sources, or they may be partly or wholly synthetically produced. Examples of antibodies of the invention are the immunoglobulin isotypes (e.g., IgG, IgE, IgM, IgD and IgA) and their isotypic subclasses; fragments which comprises an antigen binding domain such as Fab, scFv, Fv, dAb, Fd; and diabodies. Antibodies may be polyclonal or monoclonal. A monoclonal antibody may be referred to as a “mAb”.

It is possible to take monoclonal and other antibodies and use techniques of recombinant DNA technology to produce other antibodies or chimeric molecules, which retain the specificity of the original antibody. Such techniques may involve introducing DNA encoding the immunoglobulin variable region, or the CDRs, of an antibody to the constant regions, or constant regions plus framework regions, of a different immunoglobulin. See, for instance, EP-A-184187, GB 2188638A or EP-A-239400. A hybridoma or other cell producing an antibody may be subject to genetic mutation or other changes, which may or may not alter the binding specificity of antibodies produced.

As antibodies can be modified in a number of ways, the term “antibody” should be construed as covering any specific binding member or substance having a binding domain with the required specificity. Thus, this term covers antibody fragments, derivatives, functional equivalents and homologues of antibodies, humanised antibodies, including any polypeptide comprising an immunoglobulin binding domain, whether natural or wholly or partially synthetic. Chimeric molecules comprising an immunoglobulin binding domain, or equivalent, fused to another polypeptide are therefore included. Cloning and expression of chimeric antibodies are described in EP-A-0120694 and EP-A-0125023. A humanised antibody may be a modified antibody having the variable regions of a non-human, e.g., murine, antibody and the constant region of a human antibody. Methods for making humanised antibodies are described in, for example, U.S. Pat. No. 5,225,539.

It has been shown that fragments of a whole antibody can perform the function of binding antigens. Examples of binding fragments are (i) the Fab fragment consisting of VL, VH, CL and CH1 domains; (ii) the Fd fragment consisting of the VH and CH1 domains; (iii) the Fv fragment consisting of the VL and VH domains of a single antibody; (iv) the dAb fragment (Ward et al. 1989) which consists of a VH domain; (v) isolated CDR regions; (vi) F(ab′)2 fragments, a bivalent fragment comprising two linked Fab fragments; (vii) single chain Fv molecules (scFv), wherein a VH domain and a VL domain are linked by a peptide linker which allows the two domains to associate to form an antigen binding site (Bird et al. 1988; Huston et al. 1988); (viii) bispecific single chain Fv dimers (PCT/US92/09965) and; (ix) “diabodies”, multivalent or multispecific fragments constructed by gene fusion (WO94/13804; (Holliger, Prospero, and Winter 1993)).

Diabodies are multimers of polypeptides, each polypeptide comprising a first domain comprising a binding region of an immunoglobulin light chain and a second domain comprising a binding region of an immunoglobulin heavy chain, the two domains being linked (e.g., by a peptide linker) but unable to associated with each other to form an antigen binding site: antigen binding sites are formed by the association of the first domain of one polypeptide within the multimer with the second domain of another polypeptide within the multimer (WO94/13804).

Where bispecific antibodies are to be used, these may be conventional bispecific antibodies, which can be manufactured in a variety of ways (Holliger and Winter 1993), e.g., prepared chemically or from hybrid hybridomas, or may be any of the bispecific antibody fragments mentioned above. It may be preferable to use scFv dimers or diabodies rather than whole antibodies. Diabodies and scFv can be constructed without an Fc region, using only variable domains, potentially reducing the effects of anti-idiotypic reaction. Other forms of bispecific antibodies include the single chain “Janusins” described in (Traunecker, Lanzavecchia, and Karjalainen 1991).

Bispecific diabodies, as opposed to bispecific whole antibodies, may also be useful because they can be readily constructed and expressed in E. coli. Diabodies (and many other polypeptides such as antibody fragments) of appropriate binding specificities can be readily selected using phage display (WO94/13804) from libraries. If one arm of the diabody is to be kept constant, for instance, with a specificity directed against antigen X, then a library can be made where the other arm is varied and an antibody of appropriate specificity selected.

A “binding domain” is the part of a specific binding member which comprises the area, which specifically binds to and is complementary to part or all of an antigen. Where the binding member is an antibody or antigen-binding fragment thereof, the binding domain may be a CDR.

Where an antigen is large, an antibody may only bind to a particular part of the antigen, which part is termed an epitope. An antigen binding domain may be provided by one or more antibody variable domains. An antigen binding domain may comprise an antibody light chain variable region (VL) and an antibody heavy chain variable region (VH).

“Specific” is generally used to refer to the situation in which one member of a specific binding pair will not show any significant binding to molecules other than its specific binding partner(s), and, e.g., has less than about 30%, preferably 20%, 10%, or 1% cross reactivity with any other molecule. The term is also applicable where e.g., an antigen binding domain is specific for a particular epitope which is carried by a number of antigens, in which case, the specific binding member carrying the antigen binding domain will be able to bind to the various antigens carrying the epitope. Specific binding members of the invention may be capable of binding specifically to LeY in the sense that there is no detectable binding to any other antigen (such as any other glycan) when binding is tested according to the protocol set out in “Glycome Analysis” in the Examples herein.

“Isolated” refers to the state in which specific binding members of the invention or nucleic acid encoding such binding members will preferably be, in accordance with the present invention. Members and nucleic acid will generally be free or substantially free of material with which they are naturally associated such as other polypeptides or nucleic acids with which they are found in their natural environment, or the environment in which they are prepared (e.g., cell culture) when such preparation is by recombinant DNA technology practised in vitro or in vivo. Specific binding members and nucleic acid may be formulated with diluents or adjuvants and still for practical purposes be isolated—for example, the members will normally be mixed with gelatin or other carriers if used to coat microtitre plates for use in immunoassays, or will be mixed with pharmaceutically acceptable carriers or diluents when used in diagnosis or therapy. Specific binding members may be glycosylated, either naturally or by systems of heterologous eukaryotic cells, or they may be (for example if produced by expression in a prokaryotic cell) non-glycosylated.

By “substantially as set out” it is meant that the amino acid sequence(s) of the invention will be either identical or highly homologous to the amino acid sequence(s) referred to. By “highly homologous” it is contemplated that there may be from 1 to 5, from 1 to 4, from 1 to 3, 2 or 1 amino acid substitutions made in the sequence.

The invention also includes within its scope polypeptides having the amino acid sequence as set out in FIG. 2 polynucleotides having the nucleic acid sequences as set out in FIG. 2 sequences having substantial identity thereto, for example at least 70%, at least 80%, at least 85%, at least 90%, at least 95% or at least 99% identity thereto. The percent identity of two amino acid sequences or of two nucleic acid sequences is generally determined by aligning the sequences for optimal comparison purposes (e.g., gaps can be introduced in the first sequence for best alignment with the second sequence) and comparing the amino acid residues or nucleotides at corresponding positions. The “best alignment” is an alignment of two sequences that results in the highest percent identity. The percent identity is determined by comparing the number of identical amino acid residues or nucleotides within the sequences (i.e., % identity=number of identical positions/total number of positions ×100).

The determination of percent identity between two sequences can be accomplished using a mathematical algorithm known to those of skill in the art. An example of a mathematical algorithm for comparing two sequences is the algorithm of Karlin and Altschul, 1990 (Karlin and Altschul 1990), modified as in Karlin and Altschul, 1993 (Karlin and Altschul 1993). The NBLAST and XBLAST programs of Altschul et al., 1990 (Altschul et al. 1990) have incorporated such an algorithm. BLAST nucleotide searches can be performed with the NBLAST program, score=100, word length=12 to obtain nucleotide sequences homologous to a nucleic acid molecules of the invention. BLAST protein searches can be performed with the XBLAST program, score=50, word length=3 to obtain amino acid sequences homologous to a protein molecules of the invention. To obtain gapped alignments for comparison purposes, Gapped BLAST can be utilised as described in Altschul et al., 1997 (Altschul et al. 1997). Alternatively, PSI-Blast can be used to perform an iterated search that detects distant relationships between molecules (Id.). When utilizing BLAST, Gapped BLAST, and PSI-Blast programs, the default parameters of the respective programs (e.g., XBLAST and NBLAST) can be used. See http://www.ncbi.nlm.nih.gov. Another example of a mathematical algorithm utilised for the comparison of sequences is the algorithm of Myers and Miller, 1989 (Myers and Miller 1989). The ALIGN program (version 2.0) which is part of the GCG sequence alignment software package has incorporated such an algorithm. Other algorithms for sequence analysis known in the art include ADVANCE and ADAM as described in Torellis and Robotti, 1994 (Torelli and Robotti 1994); and FASTA described in Pearson and Lipman, 1988 (Pearson and Lipman 1988). Within FASTA, ktup is a control option that sets the sensitivity and speed of the search.

Isolated specific binding members of the present invention are capable of binding to a SSEA-4 carbohydrate, which may be a SSEA-4 ceramide or may be on a protein moiety. The binding domains, comprising the amino acid sequences substantially as set out as residues 105 to 116 (CDRH3) of FIG. 2 and 105 to 113 of FIG. 2, may be carried in a structure, which allows the binding of these regions to a SSEA-4 carbohydrate.

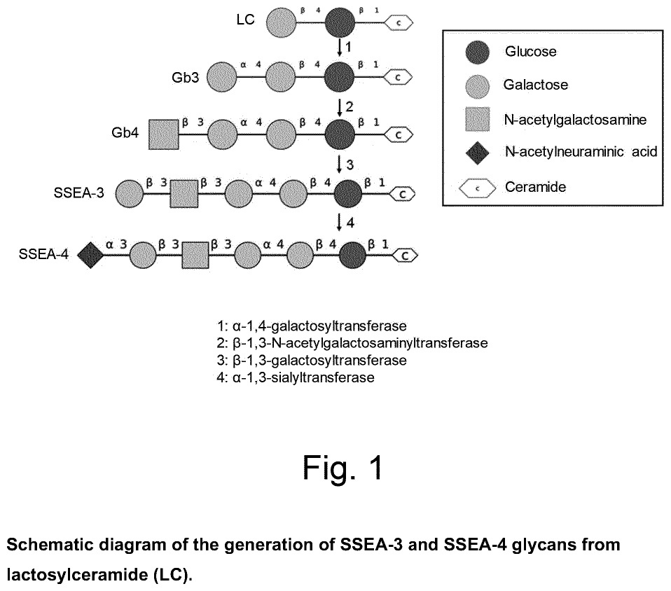

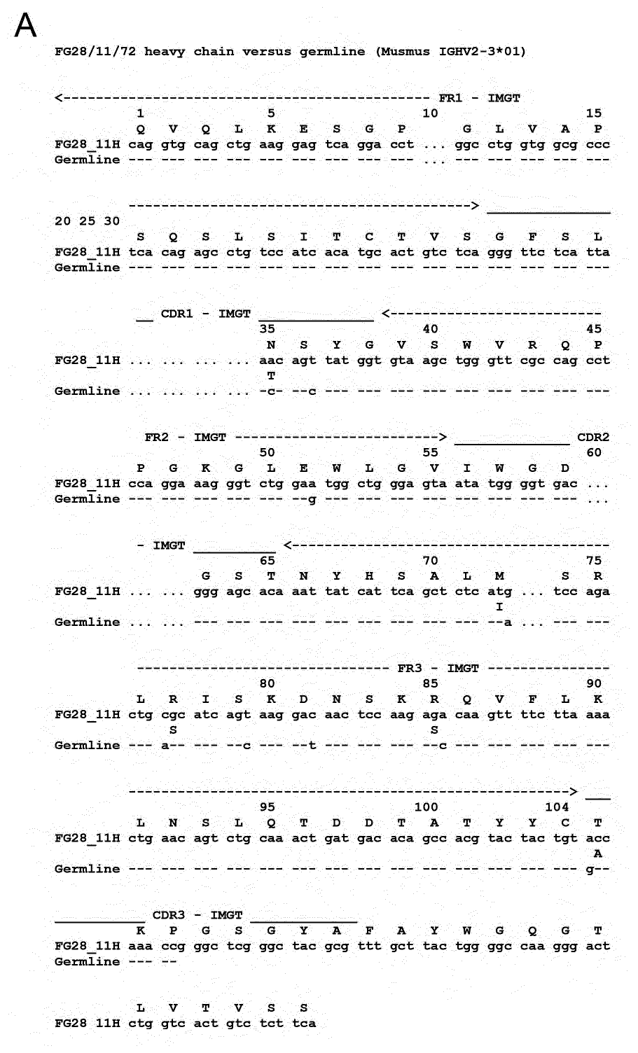

The structure for carrying the binding domains of the invention will generally be of an antibody heavy or light chain sequence or substantial portion thereof in which the binding domains are located at locations corresponding to the CDR3 region of naturally-occurring VH and VL antibody variable domains encoded by rearranged immunoglobulin genes. The structures and locations of immunoglobulin variable domains may be determined by reference to http://www.imgt.org/. The amino acid sequence substantially as set out as residues 105 to 116 of FIGS. 1a and 1b may be carried as the CDR3 in a human heavy chain variable domain or a substantial portion thereof, and the amino acid sequence substantially as set out as residues and 105 to 113 of FIG. 1c may be carried as the CDR3 in a human light chain variable domain or a substantial portion thereof.

The variable domains may be derived from any germline or rearranged human variable domain, or may be a synthetic variable domain based on consensus sequences of known human variable domains. The CDR3-derived sequences of the invention may be introduced into a repertoire of variable domains lacking CDR3 regions, using recombinant DNA technology. For example, Marks et al., 1992 (Marks et al. 1992) describe methods of producing repertoires of antibody variable domains in which consensus primers directed at or adjacent to the 5′ end of the variable domain area are used in conjunction with consensus primers to the third framework region of human VH genes to provide a repertoire of VH variable domains lacking a CDR3. Marks et al., 1992 (Marks et al. 1992) further describe how this repertoire may be combined with a CDR3 of a particular antibody. Using analogous techniques, the CDR3-derived sequences of the present invention may be shuffled with repertoires of VH or VL domains lacking a CDR3, and the shuffled complete VH or VL domains combined with a cognate VL or VH domain to provide specific binding members of the invention. The repertoire may then be displayed in a suitable host system such as the phage display system of WO92/01047 so that suitable specific binding members may be selected. A repertoire may consist of from anything from 10⁴ individual members upwards, for example from 10⁶ to 10⁸ or 10¹⁰ members.

Analogous shuffling or combinatorial techniques are also disclosed by Stemmer, 1994 (Stemmer 1994) who describes the technique in relation to a beta-lactamase gene but observes that the approach may be used for the generation of antibodies. A further alternative is to generate novel VH or VL regions carrying the CDR3-derived sequences of the invention using random mutagenesis of, for example, the FG2811VH or VL genes to generate mutations within the entire variable domain. Such a technique is described by Gram et al., 1992 (Gram et al. 1992), who used error-prone PCR.

Another method which may be used is to direct mutagenesis to CDR regions of VH or VL genes. Such techniques are disclosed by Barbas et al., 1994 (Barbas et al. 1994) and Schier et al., 1996 (Schier et al. 1996). A substantial portion of an immunoglobulin variable domain will generally comprise at least the three CDR regions, together with their intervening framework regions. The portion may also include at least about 50% of either or both of the first and fourth framework regions, the 50% being the C-terminal 50% of the first framework region and the N-terminal 50% of the fourth framework region. Additional residues at the N-terminal or C-terminal end of the substantial part of the variable domain may be those not normally associated with naturally occurring variable domain regions. For example, construction of specific binding members of the present invention made by recombinant DNA techniques may result in the introduction of N- or C-terminal residues encoded by linkers introduced to facilitate cloning or other manipulation steps, including the introduction of linkers to join variable domains of the invention to further protein sequences including immunoglobulin heavy chains, other variable domains (for example in the production of diabodies) or protein labels as discussed in more detail below.

The invention provides specific binding members comprising a pair of binding domains based on the amino acid sequences for the VL and VH regions substantially as set out in FIG. 2, i.e., amino acids 1 to 127 (VH) of FIG. 2 and amino acids 1 to 124 (VL) of FIG. 2. Single binding domains based on either of these sequences form further aspects of the invention. In the case of the binding domains based on the amino acid sequence for the VH region substantially set out in FIG. 2 such binding domains may be used as targeting agents since it is known that immunoglobulin VH domains are capable of binding target antigens in a specific manner. In the case of either of the single chain specific binding domains, these domains may be used to screen for complementary domains capable of forming a two-domain specific binding member which has in vivo properties as good as or equal to the FG2811 antibodies disclosed herein.

This may be achieved by phage display screening methods using the so-called hierarchical dual combinatorial approach as disclosed in WO92/01047 in which an individual colony containing either an H or L chain clone is used to infect a complete library of clones encoding the other chain (L or H) and the resulting two-chain specific binding member is selected in accordance with phage display techniques such as those described in that reference. This technique is also disclosed in Marks et al., 1992 (Marks et al. 1992).

Specific binding members of the present invention may further comprise antibody constant regions or parts thereof. For example, specific binding members based on the VL region shown in FIG. 2a may be attached at their C-terminal end to antibody light chain constant domains Similarly, specific binding members based on VH region shown in FIG. 2 may be attached at their C-terminal end to all or part of an immunoglobulin heavy chain derived from any antibody isotype, e.g., IgG, IgA, IgE and IgM and any of the isotype sub-classes, particularly IgG1, IgG2 and IgG4.

Specific binding members of the present invention can be used in methods of diagnosis and treatment of tumours in human or animal subjects.

When used in diagnosis, specific binding members of the invention may be labelled with a detectable label, for example a radiolabel such as ¹³¹I or ⁹⁹Tc, which may be attached to specific binding members of the invention using conventional chemistry known in the art of antibody imaging. Labels also include enzyme labels such as horseradish peroxidase. Labels further include chemical moieties such as biotin, which may be detected via binding to a specific cognate detectable moiety, e.g., labelled avidin.

Furthermore, the specific binding members of the present invention may be administered alone or in combination with other treatments, either simultaneously or sequentially, dependent upon the condition to be treated. Thus, the present invention further provides products containing a specific binding member of the present invention and an active agent as a combined preparation for simultaneous, separate or sequential use in the treatment of a tumour. Active agents may include chemotherapeutic or cytotoxic agents including, 5-Fluorouracil, cisplatin, Mitomycin C, oxaliplatin and tamoxifen, which may operate synergistically with the binding members of the present invention. Other active agents may include suitable doses of pain relief drugs such as non-steroidal anti-inflammatory drugs (e.g., aspirin, paracetamol, ibuprofen or ketoprofen) or opitates such as morphine, or anti-emetics.

Whilst not wishing to be bound by theory, the ability of the binding members of the invention to synergise with an active agent to enhance tumour killing may not be due to immune effector mechanisms but rather may be a direct consequence of the binding member binding to cell surface bound SSEA-4 glycans. Cancer immunotherapy, involving antibodies to immune checkpoint molecules, have shown effectiveness to various malignance's and in combinations with different immune-oncology treatment modalities.

Specific binding members of the present invention will usually be administered in the form of a pharmaceutical composition, which may comprise at least one component in addition to the specific binding member. The pharmaceutical composition may comprise, in addition to active ingredient, a pharmaceutically acceptable excipient, diluent, carrier, buffer, stabiliser or other materials well known to those skilled in the art. Such materials should be non-toxic and should not interfere with the efficacy of the active ingredient. The precise nature of the carrier or other material will depend on the route of administration, which may be oral, or by injection, e.g., intravenous. It is envisaged that injections will be the primary route for therapeutic administration of the compositions although delivery through a catheter or other surgical tubing is also used. Some suitable routes of administration include intravenous, subcutaneous, intraperitoneal and intramuscular administration. Liquid formulations may be utilised after reconstitution from powder formulations.

For intravenous injection, or injection at the site of affliction, the active ingredient will be in the form of a parenterally acceptable aqueous solution which is pyrogen-free and has suitable pH, isotonicity and stability. Those of relevant skill in the art are well able to prepare suitable solutions using, for example, isotonic vehicles such as Sodium Chloride Injection, Ringer's Injection, Lactated Ringer's Injection. Preservatives, stabilisers, buffers, antioxidants and/or other additives may be included, as required.

Pharmaceutical compositions for oral administration may be in tablet, capsule, powder or liquid form. A tablet may comprise a solid carrier such as gelatin or an adjuvant. Liquid pharmaceutical compositions generally comprise a liquid carrier such as water, petroleum, animal or vegetable oils, mineral oil or synthetic oil. Physiological saline solution, dextrose or other saccharide solution or glycols such as ethylene glycol, propylene glycol or polyethylene glycol may be included. Where the formulation is a liquid it may be, for example, a physiologic salt solution containing non-phosphate buffer at pH 6.8-7.6, or a lyophilised powder.

The composition may also be administered via microspheres, liposomes, other microparticulate delivery systems or sustained release formulations placed in certain tissues including blood. Suitable examples of sustained release carriers include semi-permeable polymer matrices in the form of shared articles, e.g., suppositories or microcapsules. Implantable or microcapsular sustained release matrices include polylactides (U.S. Pat. No. 3,773,919; EP-A-0058481) copolymers of L-glutamic acid and gamma ethyl-L-glutamate (Sidman et al. 1983), poly (2-hydroxyethyl-methacrylate). Liposomes containing the polypeptides are prepared by well-known methods: DE 3,218, 121A; (Eppstein et al. 1985); (Hwang, Luk, and Beaumier 1980); EP-A-0052522; EP-A-0036676; EP-A-0088046; EP-A-0143949; EP-A-0142541; JP-A-83-11808; U.S. Pat. Nos. 4,485,045 and 4,544,545. Ordinarily, the liposomes are of the small (about 200-800 Angstroms) unilamellar type in which the lipid content is greater than about 30 mol. % cholesterol, the selected proportion being adjusted for the optimal rate of the polypeptide leakage. The composition may be administered in a localised manner to a tumour site or other desired site or may be delivered in a manner in which it targets tumour or other cells.

The compositions are preferably administered to an individual in a “therapeutically effective amount”, this being sufficient to show benefit to the individual. The actual amount administered, and rate and time-course of administration, will depend on the nature and severity of what is being treated. Prescription of treatment, e.g., decisions on dosage etc., is within the responsibility of general practitioners and other medical doctors, and typically takes account of the disorder to be treated, the condition of the individual patient, the site of delivery, the method of administration and other factors known to practitioners. The compositions of the invention are particularly relevant to the treatment of existing tumours, especially cancer, and in the prevention of the recurrence of such conditions after initial treatment or surgery. Examples of the techniques and protocols mentioned above can be found in Remington's Pharmaceutical Sciences, 16^{th} edition, Oslo, A. (ed), 1980 (Remington 1980).

The optimal dose can be determined by physicians based on a number of parameters including, for example, age, sex, weight, severity of the condition being treated, the active ingredient being administered and the route of administration. In general, a serum concentration of polypeptides and antibodies that permits saturation of receptors is desirable. A concentration in excess of approximately 0.1 nM is normally sufficient. For example, a dose of 100 mg/m² of antibody provides a serum concentration of approximately 20 nM for approximately eight days.

As a rough guideline, doses of antibodies may be given weekly in amounts of 10-300 mg/m². Equivalent doses of antibody fragments should be used at more frequent intervals in order to maintain a serum level in excess of the concentration that permits saturation of the SSEA4 carbohydrate. The dose of the composition will be dependent upon the properties of the binding member, e.g., its binding activity and in vivo plasma half-life, the concentration of the polypeptide in the formulation, the administration route, the site and rate of dosage, the clinical tolerance of the patient involved, the pathological condition afflicting the patient and the like, as is well within the skill of the physician. For example, doses of 300 μg of antibody per patient per administration are preferred, although dosages may range from about 10 μg to 6 mg per dose. Different dosages are utilised during a series of sequential inoculations; the practitioner may administer an initial inoculation and then boost with relatively smaller doses of antibody.

This invention is also directed to optimised immunisation schedules for enhancing a protective immune response against cancer. The invention provides immunisation schedules for enhancing a protective immune response against cancer.

The binding members of the present invention may be generated wholly or partly by chemical synthesis. The binding members can be readily prepared according to well-established, standard liquid or, preferably, solid-phase peptide synthesis methods, general descriptions of which are broadly available (see, for example, in J. M. Stewart and J. D. Young, 1984 (Stewart and Young 1984), in M. Bodanzsky and A. Bodanzsky, 1984 (Bodanzsky and Bodanzsky 1984) or they may be prepared in solution, by the liquid phase method or by any combination of solid-phase, liquid phase and solution chemistry, e.g., by first completing the respective peptide portion and then, if desired and appropriate, after removal of any protecting groups being present, by introduction of the residue X by reaction of the respective carbonic or sulfonic acid or a reactive derivative thereof.

Another convenient way of producing a binding member according to the present invention is to express the nucleic acid encoding it, by use of nucleic acid in an expression system.

The present invention further provides an isolated nucleic acid encoding a specific binding member of the present invention. Nucleic acid includes DNA and RNA. In a preferred aspect, the present invention provides a nucleic acid, which codes for a specific binding member of the invention as defined above. Examples of such nucleic acid are shown in FIG. 2. The skilled person will be able to determine substitutions, deletions and/or additions to such nucleic acids, which will still provide a specific binding member of the present invention.

The present invention also provides constructs in the form of plasmids, vectors, transcription or expression cassettes which comprise at least one nucleic acid as described above. The present invention also provides a recombinant host cell, which comprises one or more constructs as above. As mentioned, a nucleic acid encoding a specific binding member of the invention forms an aspect of the present invention, as does a method of production of the specific binding member which method comprises expression from encoding nucleic acid. Expression may conveniently be achieved by culturing under appropriate conditions recombinant host cells containing the nucleic acid. Following production by expression, a specific binding member may be isolated and/or purified using any suitable technique, then used as appropriate.

Systems for cloning and expression of a polypeptide in a variety of different host cells are well known. Suitable host cells include bacteria, mammalian cells, yeast and baculovirus systems. Mammalian cell lines available in the art for expression of a heterologous polypeptide include Chinese hamster ovary cells, HeLa cells, baby hamster kidney cells, NS0 mouse melanoma cells and many others. A common, preferred bacterial host is E. coli. The expression of antibodies and antibody fragments in prokaryotic cells such as E. coli is well established in the art. For a review, see for example Plückthun, 1991 (Pluckthun 1991). Expression in eukaryotic cells in culture is also available to those skilled in the art as an option for production of a specific binding member, see for recent review, for example Reff, 1993 (Reff 1993); Trill et al., 1995 (Trill, Shatzman, and Ganguly 1995).

Suitable vectors can be chosen or constructed, containing appropriate regulatory sequences, including promoter sequences, terminator sequences, polyadenylation sequences, enhancer sequences, marker genes and other sequences as appropriate. Vectors may be plasmids, viral e.g., ‘phage, or phagemid, as appropriate. For further details see, for example, Sambrook et al., 1989 (Sambrook 1989). Many known techniques and protocols for manipulation of nucleic acid, for example in preparation of nucleic acid constructs, mutagenesis, sequencing, introduction of DNA into cells and gene expression, and analysis of proteins, are described in detail in Ausubel et al., 1992 (Ausubel 1992).

Thus, a further aspect of the present invention provides a host cell containing nucleic acid as disclosed herein. A still further aspect provides a method comprising introducing such nucleic acid into a host cell. The introduction may employ any available technique. For eukaryotic cells, suitable techniques may include calcium phosphate transfection, DEAE-Dextran, electroporation, liposome-mediated transfection and transduction using retrovirus or other virus, e.g., vaccinia or, for insect cells, baculovirus. For bacterial cells, suitable techniques may include calcium chloride transformation, electroporation and transfection using bacteriophage. The introduction may be followed by causing or allowing expression from the nucleic acid, e.g., by culturing host cells under conditions for expression of the gene.

The nucleic acid of the invention may be integrated into the genome (e.g., chromosome) of the host cell. Integration may be promoted by inclusion of sequences, which promote recombination with the genome, in accordance with standard techniques.

The present invention also provides a method, which comprises using a construct as stated above in an expression system in order to express a specific binding member or polypeptide as above.

Preferred features of each aspect of the invention are as for each of the other aspects mutatis mutandis. The prior art documents mentioned herein are incorporated to the fullest extent permitted by law.

In certain aspects, the disclosure provides a pharmaceutical composition comprising the mAb or binding fragment thereof described herein and a pharmaceutically acceptable carrier.

Immunomodulatory mAbs are designed to either block key inhibitory pathways suppressing effector T-cells (checkpoint blockers) or to agonistically engage costimulatory immune receptors (immunostimulatory). In this patent we have shown that 2811 mAbs can stimulate T-cell proliferation in vitro and in vivo. Isotype-dependent FcγRIIB engagement has been shown to be requisite for the activity of immune-agonistic mAbs. These agents stimulate signaling through their target receptors, typically members of the tumor necrosis factor receptor (TNFR) superfamily, whilst receptor clustering and ensuing downstream signalling is promoted by mAb Fc interactions with FcγRIIB. As cancer therapeutics, they are designed to enhance tumor immunity by engaging costimulatory receptors such as CD40, 4-1BB, or OX40, on APC or T-effector cells, or to promote apoptosis by stimulating death receptors (DRs) such as DR4, DRS, or Fas (CD95) on cancer cells. In contrast to direct targeting agents, the agonistic activity of these mAbs is dependent on their ability to engage inhibitory FcγRIIB and mAbs with high ratios of binding to activating rather than inhibitory receptors (A:I) (e.g., mouse IgG2a, human IgG1) are largely inactive in preclinical models, whereas those with low A:I ratios (eg, mouse IgG1 and hIgG2) are highly agonistic. Signaling through FcγRIIB is not required to confer activity; rather, it provides a crosslinking scaffold for the mAbs to facilitate TNFR clustering and activation (Beers, Glennie, and White 2016). In this regard FG2811 mIgG1 was used in vivo to stimulate TSCMs, whereas plate-bound 2811 hIgG1 or mIgG3 could be used in vitro. An alternative approach constitutes the use of the hIgG2 isotype. This human isotype, with limited binding affinity for FcγRIIB, possesses the intrinsic ability to drive receptor clustering, through its unique hinge disulfide configuration (White 2015; Liu 2019; Yu 2020). On synthesis, hIgG2 converts to a range of isoforms through disulfide bond rearrangement of its hinge and CH1 domains, with the more compact and rigid form displaying potent in vitro and in vivo FcγRIIB-independent receptor clustering. Accordingly, we show 2811 hIgG2-induced stimulation of TSCMs

In some aspects, the invention provides a method of isolating stem memory T-cells (T_{SCM}) ex vivo via binding of an isolated specific binding member of the invention to the SSEA-4 antigen. In some aspects, the invention provides a method of proliferating stem memory T-cells (T_{SCM}) ex vivo via binding of an isolated specific binding member of the invention to the SSEA-4 antigen. In some aspects, the invention provides a method of isolating and proliferating stem memory T-cells (T_{SCM}) ex vivo via binding of an isolated specific binding member of the invention to the SSEA-4 antigen.

In certain aspects, the invention provides a method of isolating and/or proliferating stem memory T-cells (T_{SCM}) ex vivo using mAb 2811 of any mouse or human isotype. In certain aspects, the invention provides a method of isolating and/or proliferating stem memory T-cells (T_{SCM}) ex vivo using an isolated antibody or a binding fragment or member thereof comprising the binding domains of mAb 2811 and any framework region from any mouse or human antibody isotype.

In some aspects, the invention provides a method of isolating stem memory T-cells (T_{SCM}) in vivo via binding of an isolated specific binding member of the invention to the SSEA-4 antigen. In some aspects, the invention provides a method of proliferating stem memory T-cells (T_{SCM}) in vivo via binding of an isolated specific binding member of the invention to the SSEA-4 antigen. In some aspects, the invention provides a method of isolating and proliferating stem memory T-cells (T_{SCM}) in vivo via binding of an isolated specific binding member of the invention to the SSEA-4 antigen.

In some aspects, the invention provides an isolated specific binding member of the invention for use in a method of isolating stem memory T-cells (T_{SCM}) in vivo via binding of to the SSEA-4 antigen. In some aspects, the invention provides an isolated specific binding member of the invention for use in a method of proliferating stem memory T-cells (T_{SCM}) in vivo via binding of to the SSEA-4 antigen. In some aspects, the invention provides an isolated specific binding member of the invention for use in a method of isolating and proliferating stem memory T-cells (T_{SCM}) in vivo via binding of to the SSEA-4 antigen.

In certain aspects, the invention provides a method of isolating and/or proliferating stem memory T-cells (T_{SCM}) in vivo using mAb 2811 of any mouse or human isotype. In certain aspects, the invention provides a method of isolating and/or proliferating stem memory T-cells (T_{SCM}) in vivo using an isolated antibody or a binding fragment or member thereof comprising the binding domains of mAb 2811 and any framework region from any mouse or human antibody isotype.

In some aspects of the invention proliferating stem memory T-cells (T_{SCM}) refers to increasing the expansion of the cells and/or promoting cell division.

In some aspects of the invention, the cells are identified or purified by using a binding member of the invention to target or label the cell and then applying a cell sorting or cell separation method. In some aspects of the invention the binding member of the invention can be used with cell sorting or cell separation methods such as fluorescence activated cell sorting (FACS), flow cytometry, Immunomagnetic Cell Separation, Immunodensity Cell Separation, Immunoguided Laser Capture Microdissection. In a preferred embodiment the binding member of the invention can be used with fluorescence activated cell sorting (FACS). In a preferred embodiment the binding member of the invention can be used with flow cytometry methods.

In certain aspects, the invention provides a method of treating cancer in a subject in need thereof, wherein the method comprises administering to the subject a therapeutic effective amount of a pharmaceutical composition comprising an isolated specific binding member of the invention. In some methods of the invention the administered binding member stimulates proliferation of isolated specific binding member of the invention in the subject.

In certain embodiments, the method provided treats cancer selected from the group consisting of brain cancer, lung cancer, breast cancer, oral cancer, oesophageal cancer, stomach cancer, liver cancer, bile duct cancer, pancreatic cancer, colon cancer, kidney cancer, bone cancer, skin cancer, cervical cancer, ovarian cancer, and prostate cancer.

In certain aspects, the invention provides a pharmaceutical composition comprising an isolated specific binding member of the invention for use in a method of treating cancer in a subject in need thereof, wherein the method comprises administering to the subject a therapeutic effective amount of the pharmaceutical composition. In some methods of the invention the administered binding member stimulates proliferation of isolated specific binding member of the invention in the subject.

In certain embodiments, the pharmaceutical composition for use according to methods of the invention treats cancer selected from the group consisting of brain cancer, lung cancer, breast cancer, oral cancer, oesophageal cancer, stomach cancer, liver cancer, bile duct cancer, pancreatic cancer, colon cancer, kidney cancer, bone cancer, skin cancer, cervical cancer, ovarian cancer, and prostate cancer.

The details of one or more embodiments of the invention are set forth in the description below. Other features or advantages of the present invention will be apparent from the following drawings and detailed description of several embodiments, and also from the appending claims.

### BRIEF DESCRIPTION OF THE DRAWINGS

As used herein, symbolic, graphic, and text nomenclature for describing glycans and related structures are well established and understood in the art, including, for example “Symbols Nomenclatures for Glycan Representation” by Ajit Varki et al (Varki et al. 2009).

### FIGURE LEGENDS

FIG. 1: Schematic diagram of the generation of SSEA-3 and SSEA-4 glycans from lactosylceramide (LC). The LMTK mouse fibroblast cell line was transduced with A4GALT, B3GALNT1 and B3GALT5 genes, which generated α-1,4-galactosyltransferase, β-1,3-N-acetylgalactosaminyltransferase and β-1,3-galactosyltransferase, respectively that added glycans to LC sequentially to make SSEA-3 and SSEA-4 glycans.

FIG. 2: Amino acid and nucleotide sequence of the FG2811 IgG3 heavy and kappa light chain variable regions and mIgG1, hIgG1 and hIgG2 constant regions.
(A) Nucleotide and amino acid sequence of the mature FG2811 heavy chain variable region, showing framework regions (FR) 1 to 3 and complementarity determining regions (CDR) 1 to 3.
(B) Nucleotide and amino acid sequence of the mature FG2811 kappa chain variable region, showing framework regions (FR) 1 to 3 and complementarity determining regions (CDR) 1 to 3.
(C) Nucleotide and amino acid sequence of mIgG1 aligned with germline
(D) Nucleotide and amino acid sequence of hIgG2 aligned with germline
(E) Nucleotide and amino acid sequence of hIgG1 aligned with germline

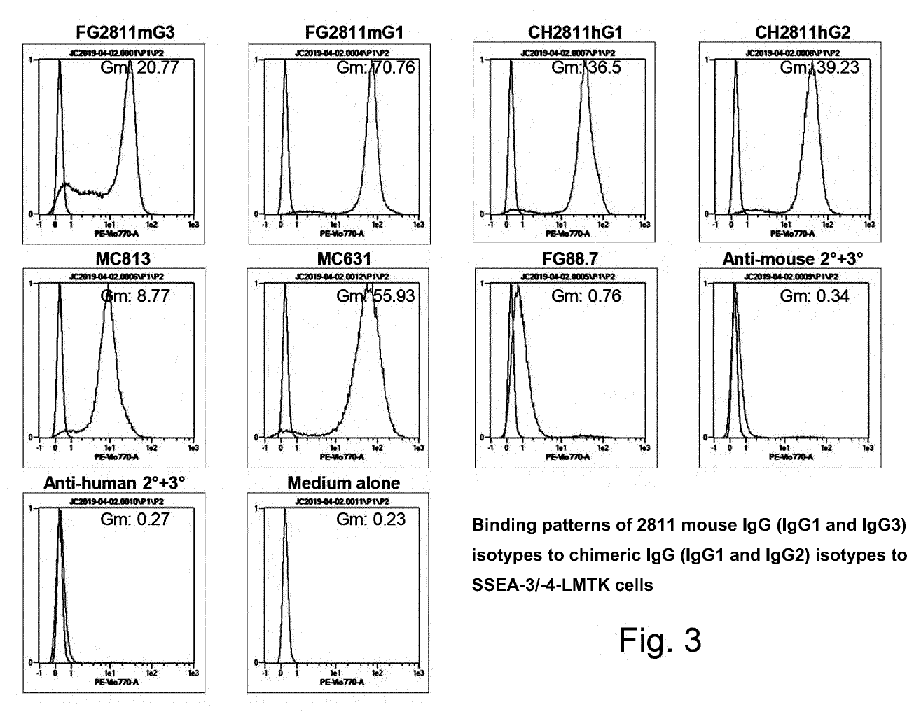

FIG. 3: Binding patterns of 2811 mouse IgG (IgG1 and IgG3) isotypes to chimeric IgG (IgG1 and IgG2) isotypes to SSEA-3/-4-LMTK cells.

The binding of FG2811mG3, FG2811mG1, CH2811hG1, CH2811hG2, MC813 (anti-SSEA-4 mAb; mouse IgG1), MC631 (anti-SSEA-3 mAb; rat IgM), FG88.7 (anti-Lewis^{a/c/x} mAb; mouse IgG3), anti-mouse secondary and tertiary antibody alone, anti-human secondary and tertiary antibody alone and medium alone to SSEA-3/-4-LMTK cells was assessed by flow cytometry. The result was presented as geometric mean (Gm) values.

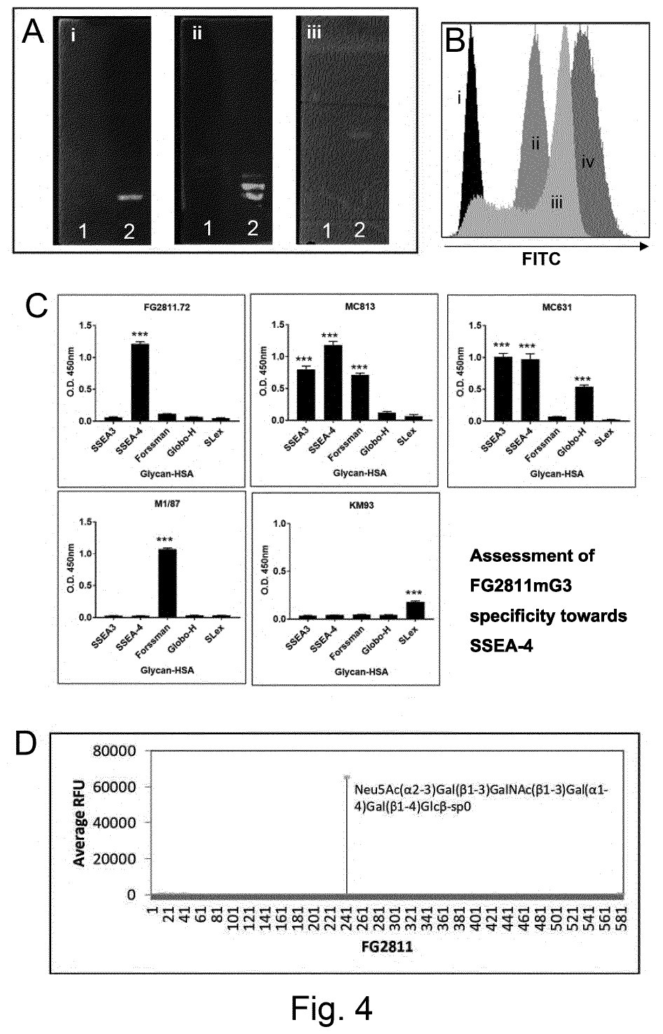

FIG. 4: Assessment of FG2811mG3 specificity towards SSEA-4.
(A) Binding of FG2811mG3 mAb to lipid antigens as assessed by HPTLC. Thin layer chromatography analysis of 1) wild type LMTK and 2) SSEA-3/-4-LMTK plasma membrane lipid extracts using i) FG2811mG3 mAb, ii) MC631 mAbs and iii) MC813 mAbs at 5 μg/ml;
(B) FG2811mG3 mAb bound to SSEA-3/-4-LMTK cell surface antigens. Binding of i) secondary antibody alone, ii) MC813, iii) FG2811mG3 and iv) MC631 at 5 μg/ml to SSEA-3/-4-LMTK cells were assessed by direct immunofluorescence staining and flow cytometry analysis. Results were expressed as Gm values;
(C) Reactivity of FG2811mG3 mAb with HSA-coupled glycan antigens (SSEA-3, SSEA-4, Globo-H and Forssman). Binding of i) FG2811mG3, ii) MC813, iii) MC631, iv) M1/87 and v) KM93 mAbs at 5 μg/ml to HSA-coupled glycans was assessed by ELISA. MC631 (anti-SSEA-3 and SSEA-4), MC813 (anti-SSEA-4), M1/87 (anti-Forssman) and KM93 (anti-sialyl-Lewis^{x}) were included as positive control mAbs. Antibody activity was measured by absorbance at 450 nm. Error bars representing the mean±standard deviation of quadruplicate wells (*** p<0.0001; * p<0.05 versus control, ANOVA followed by the Bonferroni multiple comparisons test, GraphPad Prism 6);
(D) Binding of FG2811mG3 mAb to Consortium for Functional Glycomics glycan array. (CFG, core H, version 5.1). Sp denotes the length of spacer between the glycans on the slide.

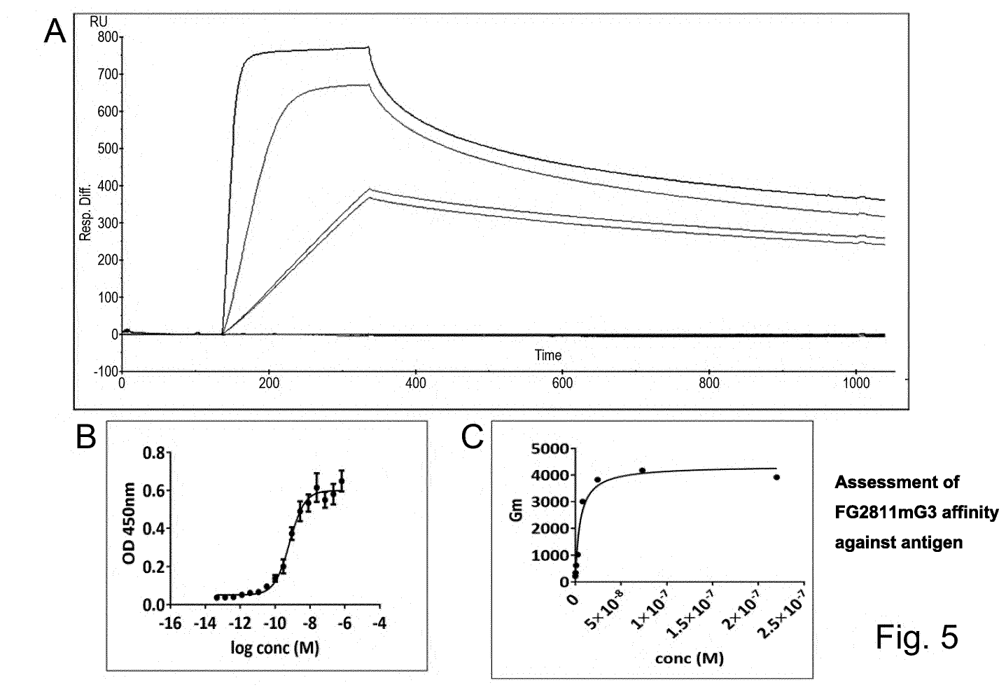

FIG. 5: Assessment of FG2811mG3 affinity against antigen.
(A) SSEA-3/-4-LMTK plasma membrane lipid antigen binding kinetics of FG2811mG3 mAb was examined using SPR (Biacore X);
(B) SSEA-3/-4-LMTK plasma membrane lipid ELISA. A concentration range of the FG2811mG3 mAb was incubated in SSEA-3/-4-LMTK plasma membrane lipid-coated microwells. EC₅₀ values (6.8×10⁻¹⁰ M) were obtained via non-linear regression on log-transformed data (GraphPad Prism 6);
(C) SSEA-3/-4-LMTK cell surface binding. SSEA-3/-4-LMTK cells were incubated with a concentration ranged of FG2811mG3 mAb and cell binding analysed by flow cytometry. Fitting of the background—subtracted data to a one site specific binding model (GraphPad Prism 6) generated the K_{d} values. Representative binding curves from three independent experiments are shown.

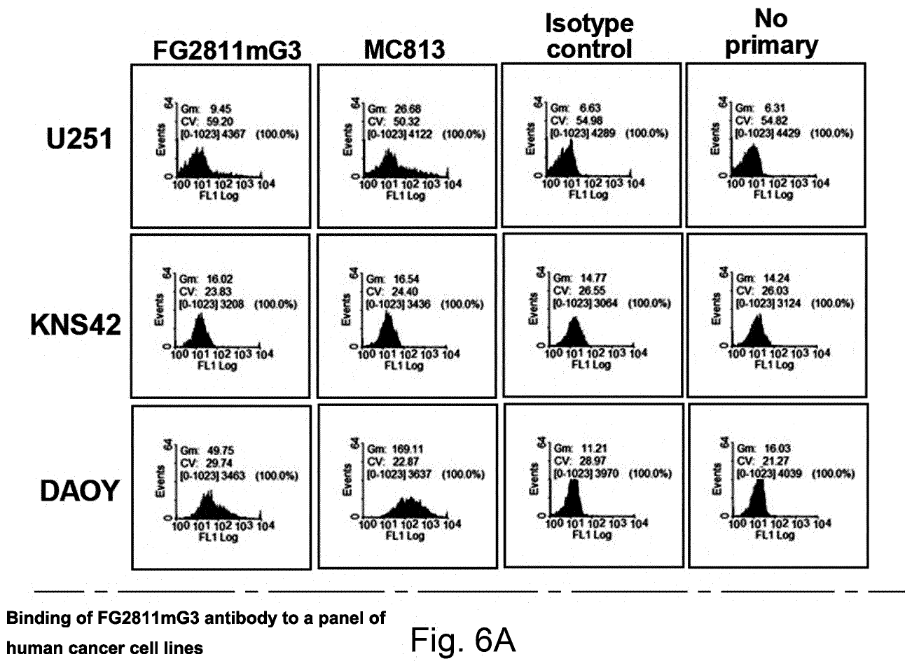

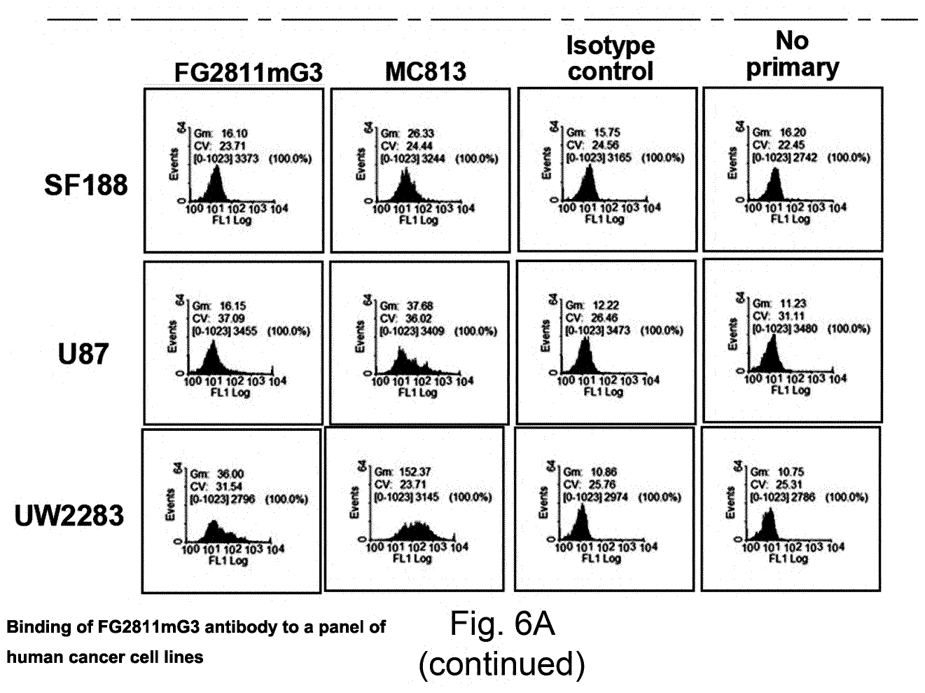

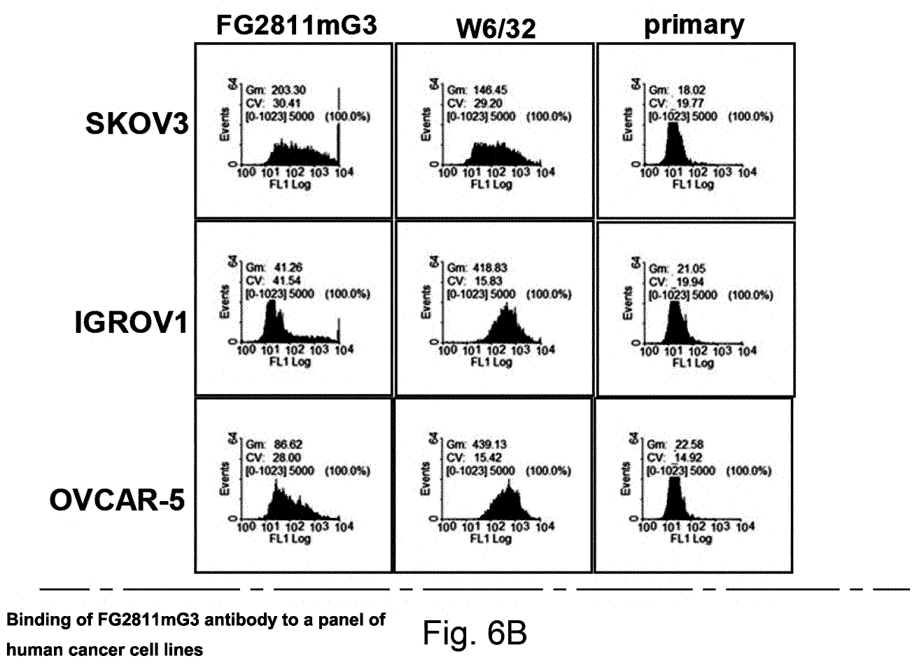

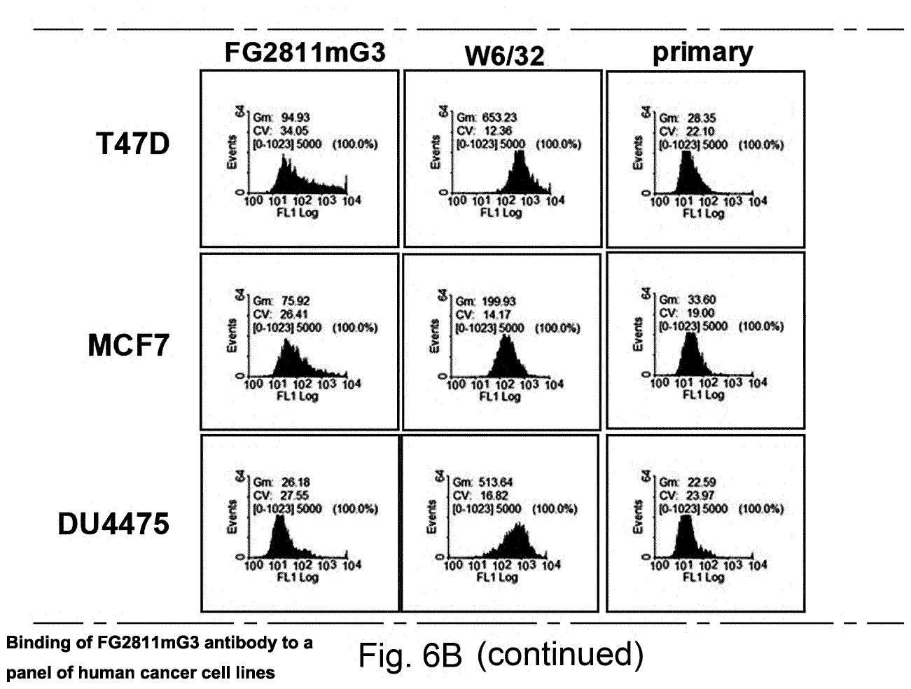

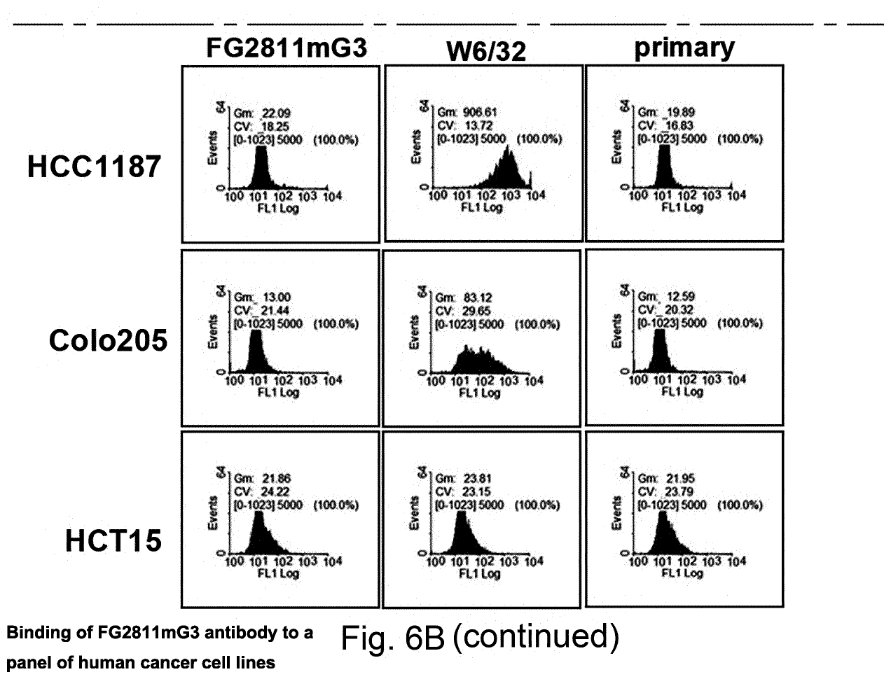

FIG. 6: Binding of FG2811mG3 antibody to a panel of human cancer cell lines.
(A) Antibody binding to brain cancer cell lines (U251, KNS42, DAOY, SF188, U87 and UW2283). Antibody FG2811mG3, MC813, mouse IgG3 kappa isotype control and secondary antibody alone (no primary) binding to a brain cancer cell lines at 5 μg/ml was assessed by flow cytometry and result presented as Gm values;
   (B) Antibody binding to ovarian (SKOV3, IGROV1 and OVCAR-5), breast (T47D, MCF7, DU4475 and HCC1187) and colorectal (Colo205 and HCT15). Antibody FG2811mG3, anti-HLA-A, B, C (W6/32) and secondary antibody alone (no primary) binding to a panel of cancer cell lines at 5 μg/ml was assessed by flow cytometry and result presented as Gm values.

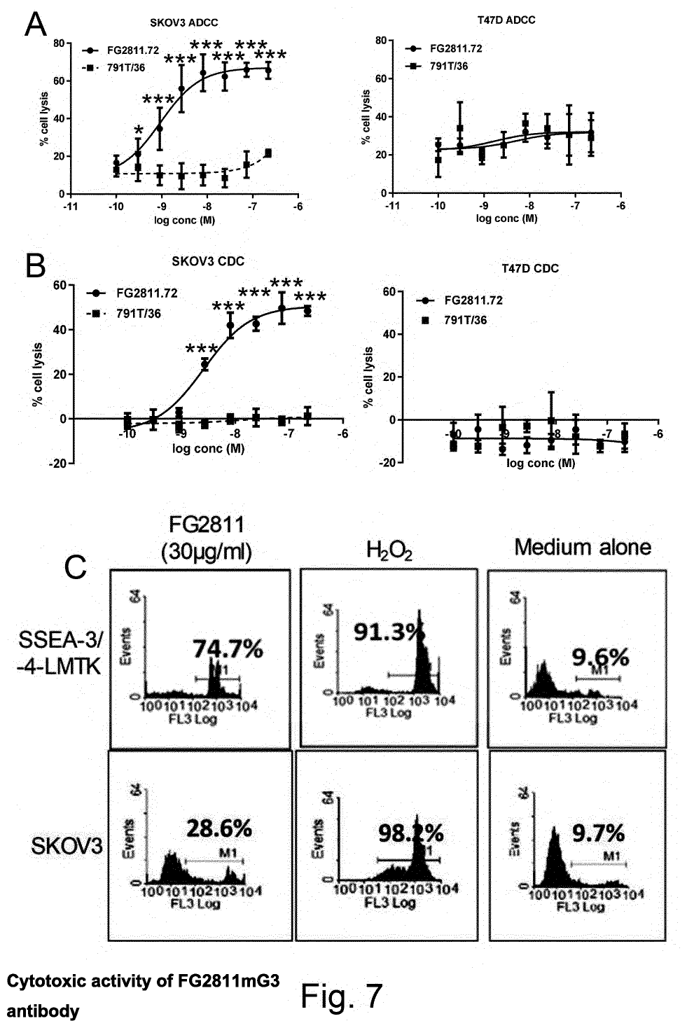

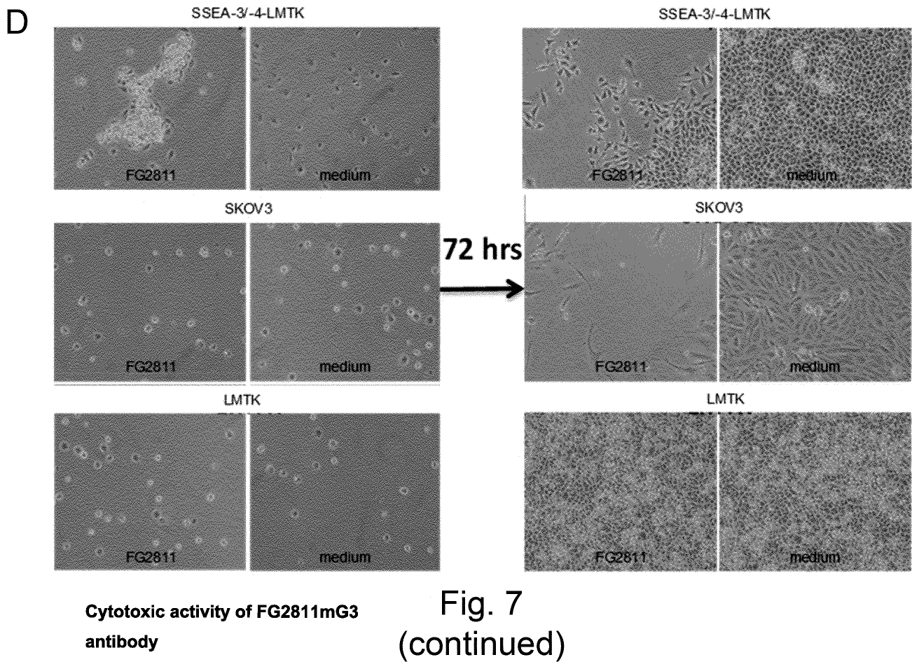

FIG. 7: Cytotoxic activity of FG2811mG3 antibody.
(A) ADCC killing of cancer cells by FG2811mG3 mAb. Dose-dependent ADCC activity of FG2811mG3 mAb on SKOV3 and T47D cells. The ⁵¹Cr-labelled cancer target cells were co-incubated with increasing concentrations of FG2811mG3 mAb (0.003-10 μg/ml) and human PBMCs (target cells: PBMCs; 100:1). The ⁵¹Cr released into supernatant was measured and expressed as the percentage of total ⁵¹Cr released with 10% Triton-X. The anti-CD55 mAb (791T/36) was used as negative control mAb. Significance versus PBMC control was established by ANOVA followed by Bonferroni multiple comparison test, GraphPad Prism 6. (***, P<0.001 versus control);
(B) CDC killing of cancer cells by FG2811mG3 mAb. Dose-dependent CDC activity of FG2811mG3 mAb on SKOV3 and T47D cells. The ⁵¹Cr-labelled cancer target cells were co-incubated with increasing concentrations of FG2811mG3 mAb (0.003-10 μg/ml) and human serum. The ⁵¹Cr released into supernatant was measured and expressed as the percentage of total ⁵¹Cr released with 10% Triton-X. The anti-CD55 mAb (791T/36) was used as negative control mAb. Significance versus PBMC control was established by ANOVA followed by Bonferroni multiple comparison test, GraphPad Prism 6. (*, P<0.005; ***, P<0.001 versus control);
(C) FG2811mG3 induced direct cell death of cancer cells at 37° C. Propidium iodide (PI) uptake following mAb exposure was assessed by flow cytometric analysis. SSEA-3/-4-LMTK cells were incubated with 30 μg/ml of FG2811mG3 mAb at 37° C. Hydrogen peroxide (H₂O₂) and medium alone were included as positive and negative controls, respectively;
(D) Phase contrast imaging of FG2811mG3-treated cancer cells. Images (magnification ×10) showing SSEA-3/-4-LMTK, SKOV3 and LMTK cells after incubation with FG2811mG3 mAb at 30 μg/ml and medium alone for 72 hrs.

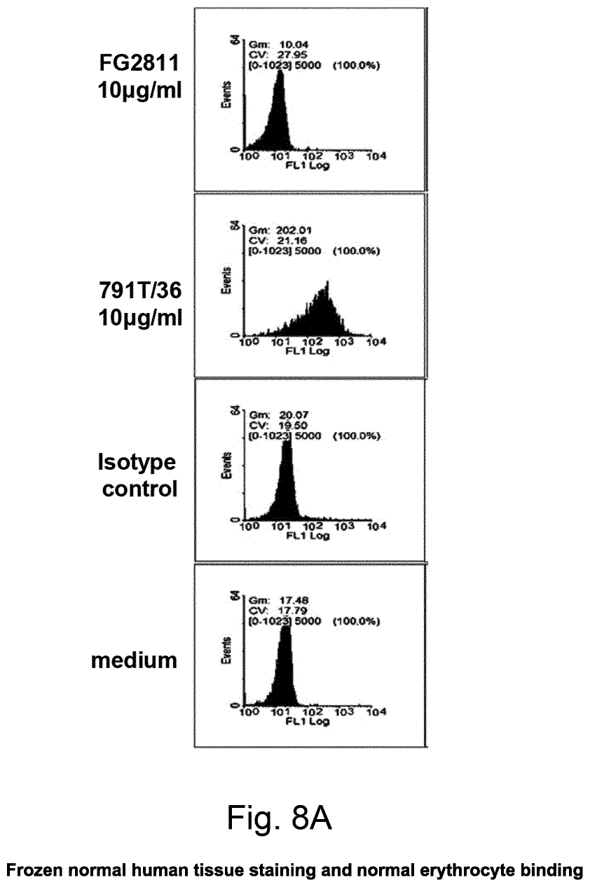

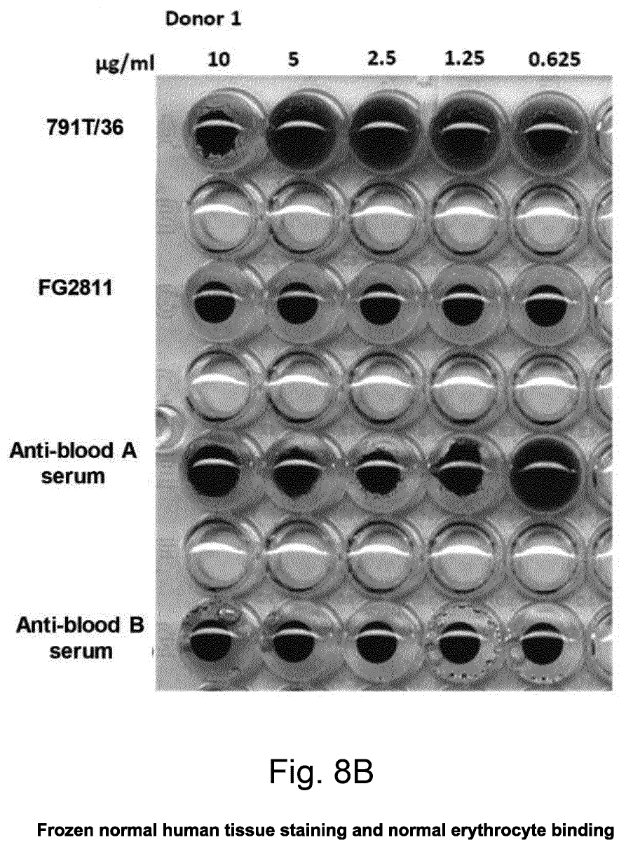

FIG. 8: Normal erythrocyte binding.
(A) Evaluation of FG2811mG3 mAb binding to healthy donor erythrocytes by flow cytometry. Erythrocyte binding by FG2811mG3 mAb was compared to 791T/36 positive control mAb (anti-CD55 mAb) by flow cytometry. Both mAbs were used at 10 μg/ml. The isotype control mAb and medium alone were used as negative controls. Result representative of 5 donors;
(B) Hemagglutination assay. Erythrocytes agglutination by FG2811mG3 mAb at various concentrations (0.625 to 10 μg/ml) was compared to 791T/36 and anti-blood group positive control antibodies. PBS was used as negative control. Results are representative of 5 donors.

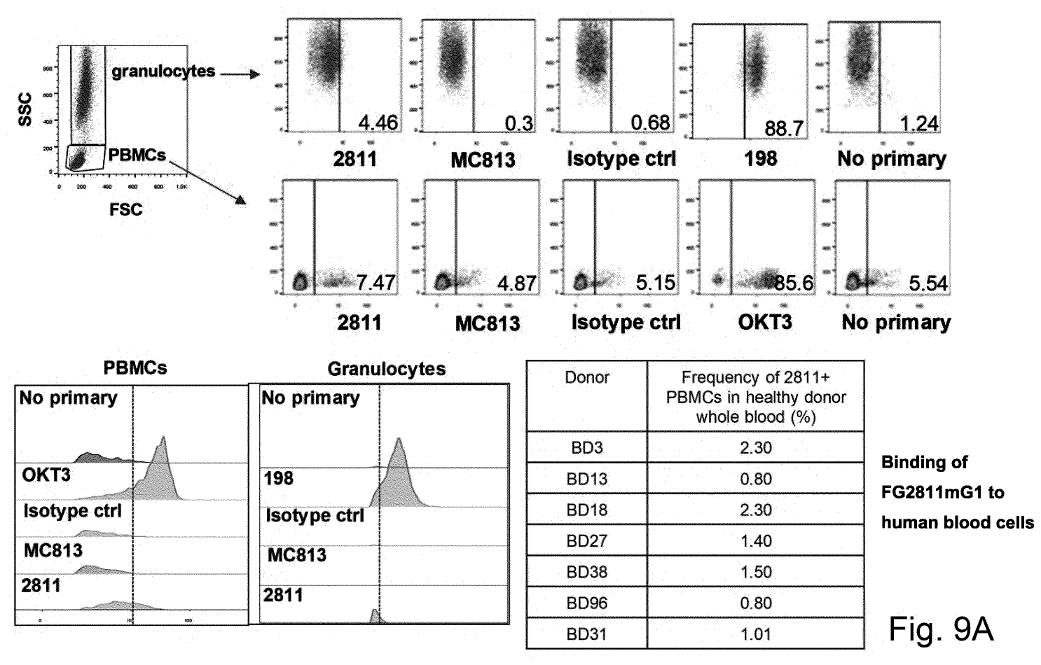

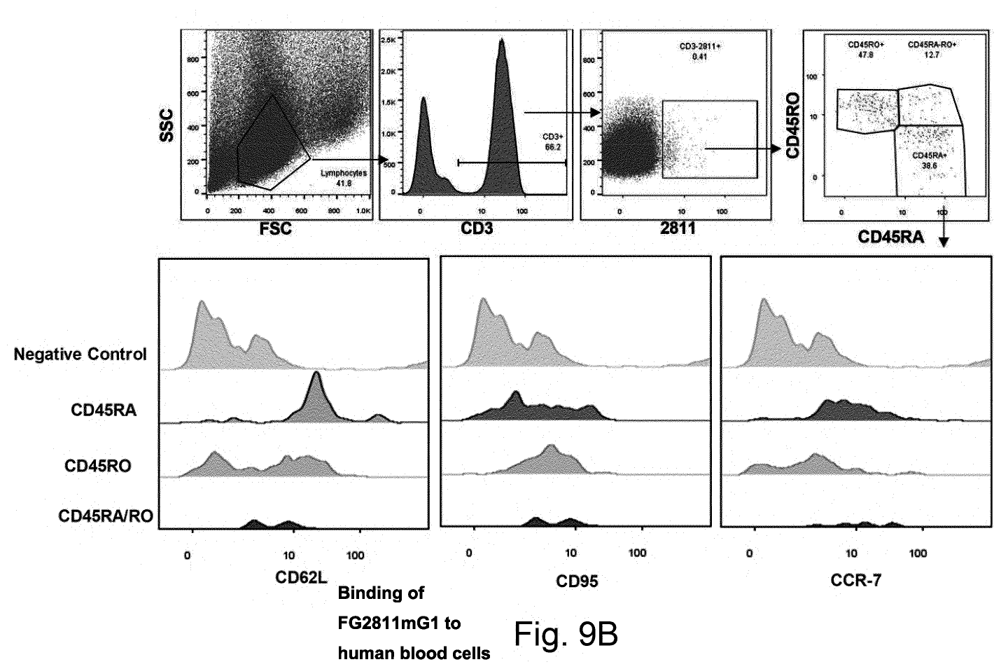

FIG. 9: Binding of FG2811mG1 to human blood cells.
(A) FG2811mG1 bound to PBMCs of whole blood from healthy donors. Binding of healthy donor whole blood with FG2811mG1, MC813, mouse IgG1 isotype control antibody (isotype ctrl), OKT3 (anti-CD3), 198 (anti-CEACAM6) and anti-mouse IgG Fc specific-FITC secondary antibody alone (no primary) were assessed by indirect immunofluorescence staining and flow cytometric analysis. All mAbs were used at 5 μg/ml. The result shown is representative of 7 different healthy donor whole bloods. Results shown in dot plots and histograms;
(B) PBMCs phenotyping. Successive panels depicting the flow cytometric gating strategy used to phenotype CD3⁺FG2811mG3⁺ PBMCs. Gates were drawn for analysis on CD3⁺FG2811mG3⁺ cells; CD3⁺FG2811mG3⁺ cells were checked for CD45RA and CD45RO expression. The CD45RA⁺, CD45RA⁺RO⁺ and CD45RO⁺ cells were further checked for the expression of CD62L, CD95 and CCR-7 markers.

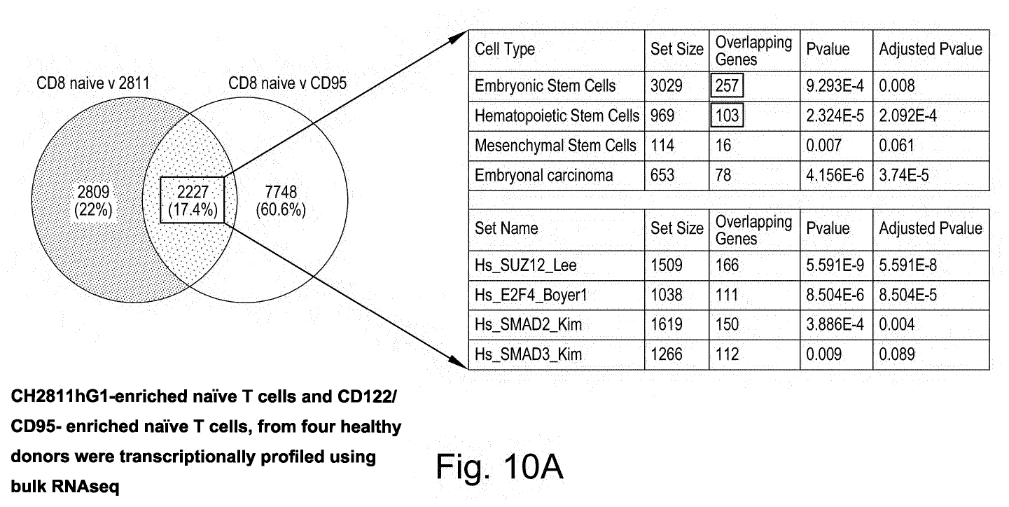

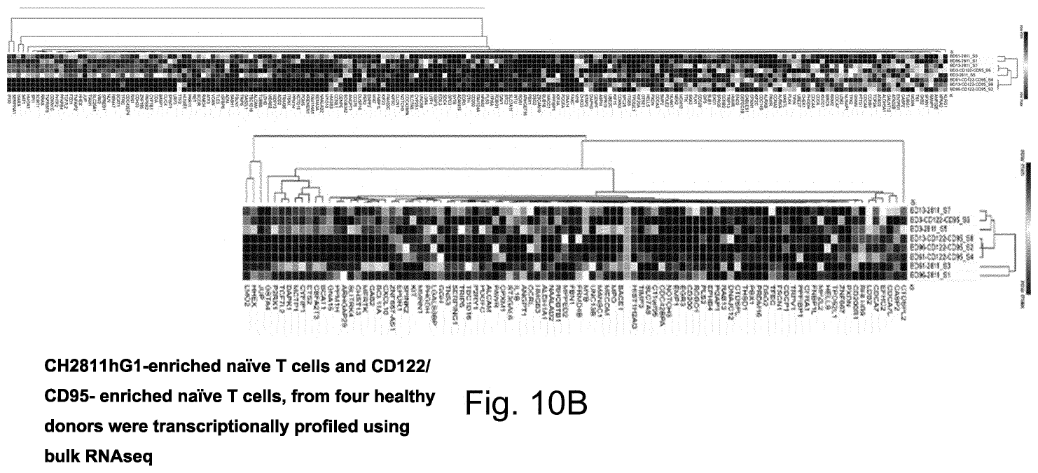

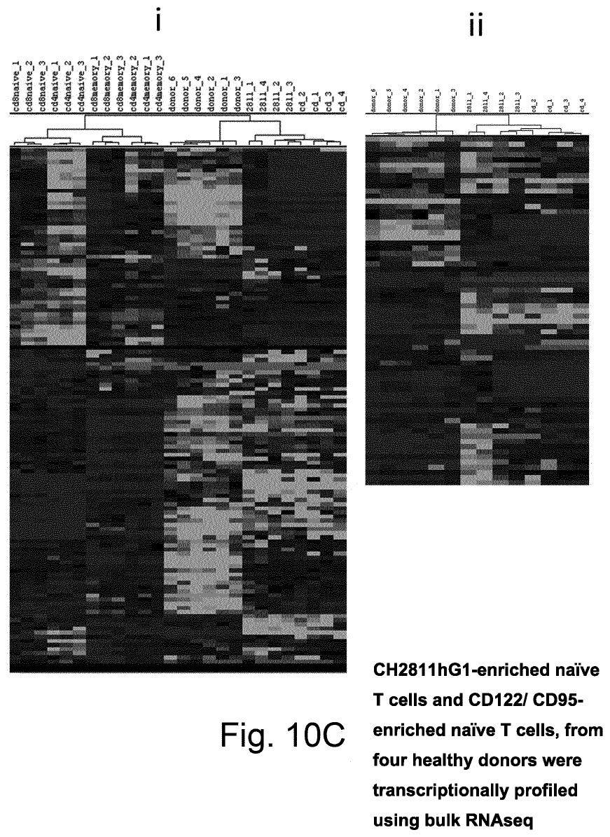

FIG. 10: CH2811hG1-enriched naïve T-cells and CD122/CD95-enriched naïve T-cells, from four healthy donors (BD3, BD13, BD61, BD96) were transcriptionally profiled using bulk RNAseq.
(A) Venn diagram showing the common genes between the two sets of differentially expressed (DE) genes obtained through comparing naïve CD8 T-cells (GSE83808) with CH2811hG1-enriched naïve T-cells and CD122/CD95-enriched naïve T-cells, respectively. The identified 2227 common genes were analysed for stemness signatures using StemChecker (Pinto et al. 2015). Statistically significant enrichment for genes associated with stem cell subsets as well as significantly enriched targets of stemness-associated transcription factors are shown in the tables;
(B) Heatmaps and hierarchical clustering (Euclidean distance) of CH2811hG1-enriched and CD122/CD95-enriched transcriptomic profiles, based on the 257 overlapping genes from the ESC and 113 from the HSC (both from tables in A), respectively. No clear segregation of the two enriched populations, suggesting commonalities in their stemness profile;
(C) (i) Heatmap based on DE genes (>2-fold, p<0.001) between CD8+T_{SCM} and T_{N} from Gattinoni et al., 2011 (Gattinoni et al. 2011) and based on (ii) a subset of transcription factors, effector function, exhaustion and homing adhesion genes shown to relate to effector differentiation from Pilipow et al., 2018 (Pilipow et al. 2018). Donor 1-6 was from GSE114765, CD8/CD4 naïve and memory datasets were from GSE23321.

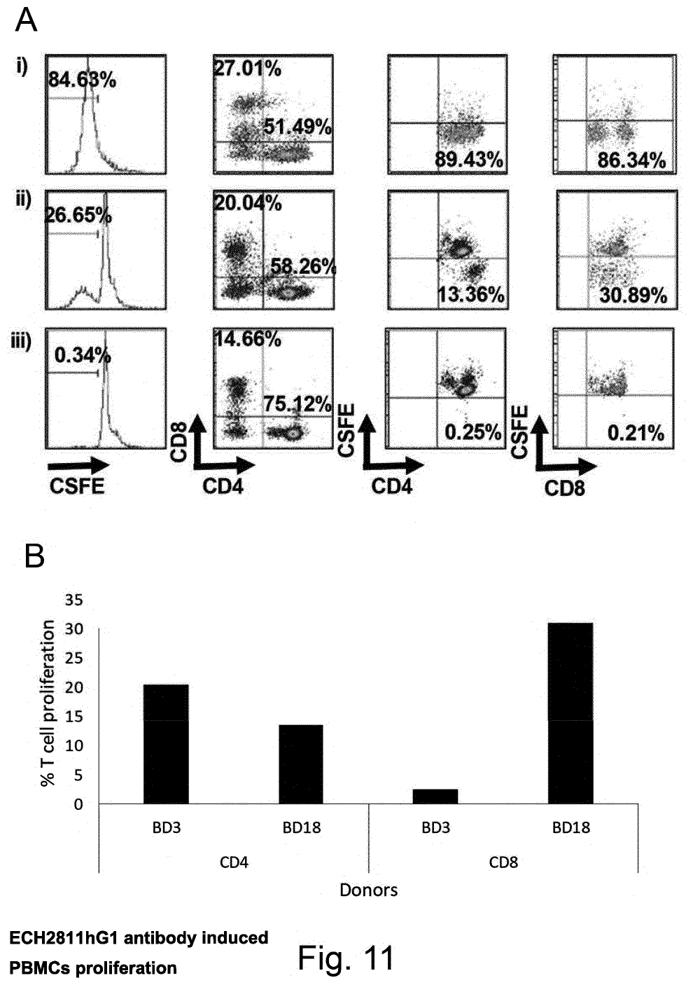

FIG. 11: CH2811hG1 antibody induced PBMCs proliferation
(A) PBMCs were isolated from two healthy donor (BD3 and BD18) whole bloods and labelled with CSFE dye. CSFE labelled T-cells from healthy donors were stimulated with plate bound i) PHA, ii) CH2811hG1 mAb and iii) medium, and cells were collected at day 11 to check for CD4 and CD8 T-cell proliferation. Percentages of specific T-cell population proliferation were assessed via CSFE dye dilution analysis. Results representative of 2 donors;
(B) Summary of CD4 and CD8 PBMCs proliferation.

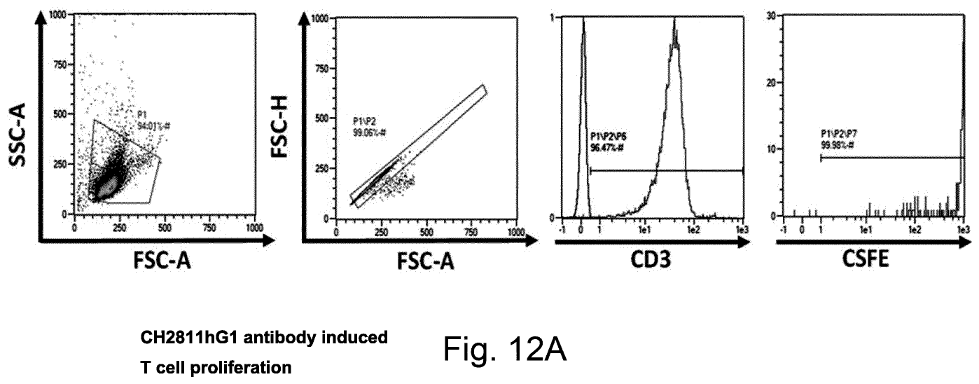

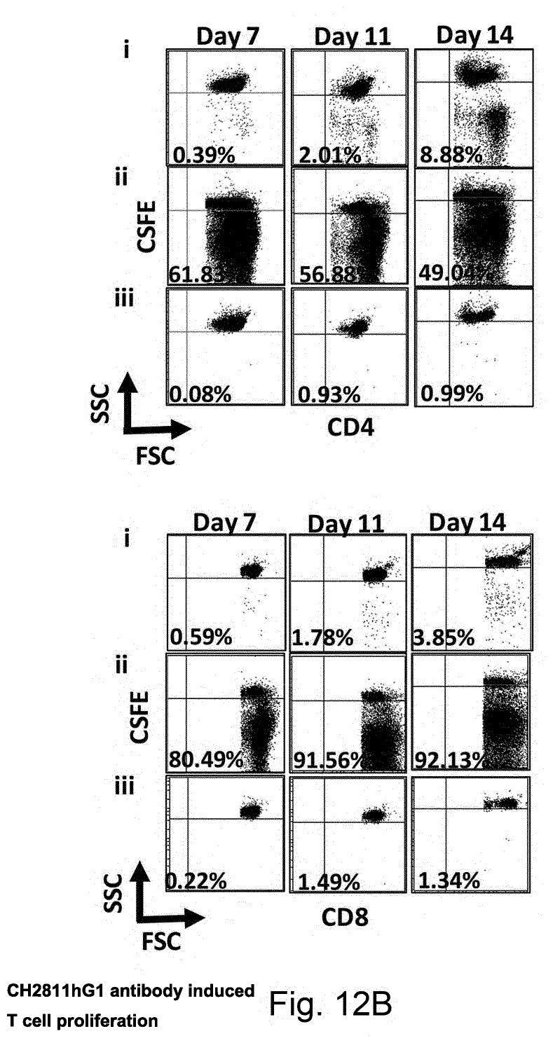

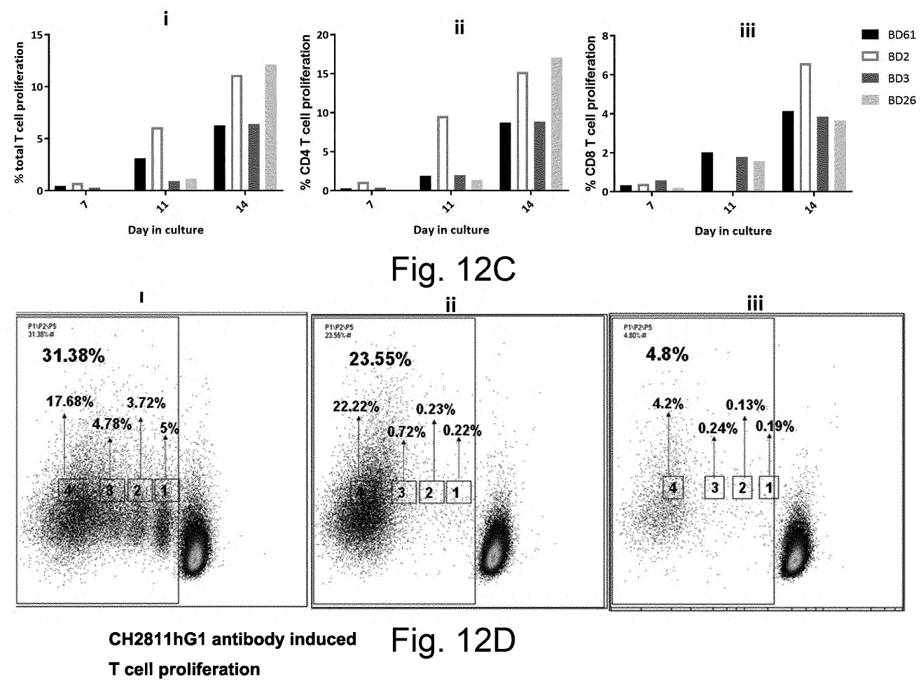

FIG. 12: CH2811hG1 antibody induced T-cell proliferation.
(A) T-cell purity and CSFE label check. Pure T-cells were isolated from four healthy donor (BD61, BD2, BD3, BD26) whole bloods and labelled with CSFE dye. T-cell purity were checked by staining T-cells with anti-CD3 antibody and CSFE labelling were checked at FITC channel;
(B) CH2811hG1 plate bound antibody induced T-cell proliferation at 5 μg/ml. CSFE labelled T-cells from healthy donors were stimulated with plate bound i) CH2811hG1, ii) anti-CD3 antibody and iii) medium and cells were collected at day 7, 11 and 14 to check for CD4 and CD8 T-cell proliferation. Percentages of specific T-cell population proliferation were assessed via CSFE dye dilution analysis. Results representative of 4 donors;
(C) Summary of i) total T-cell proliferation, ii) CD4 T-cell proliferation and iii) CD8 T-cell proliferation from 4 healthy donors (BD61, BD2, BD3, BD26), as assessed by CSFE dye dilution analysis;
(D) Percentages of CH2811hG1 stimulated CD4 T-cells at a particular number of divisions after 11 days in vitro. T-cells were loaded with CSFE dye and stimulated with i) anti-CD3, ii) CH2811hG1 or iii) medium at day 0. At day 11, the CSFE profiles were analysed by flow cytometry. The cell division times are indicated in square box and the percentage of cells that had divided certain times is indicated above the square box.

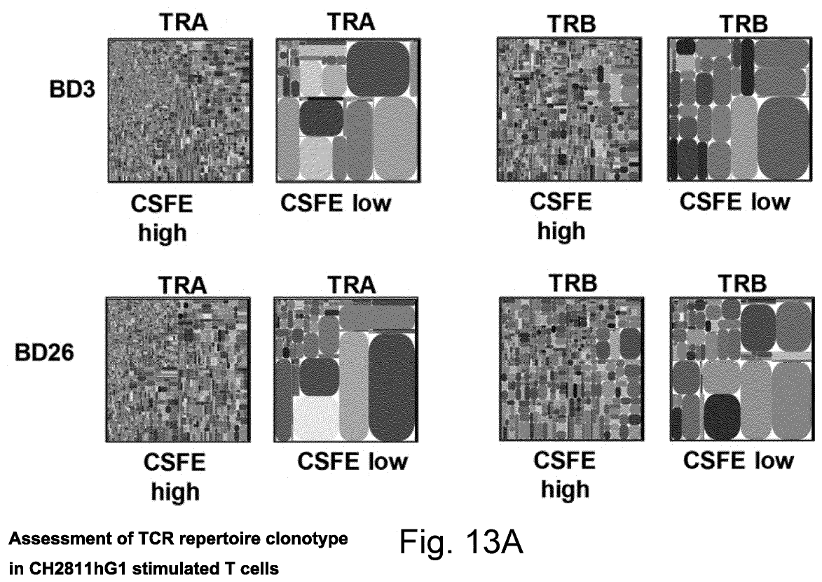

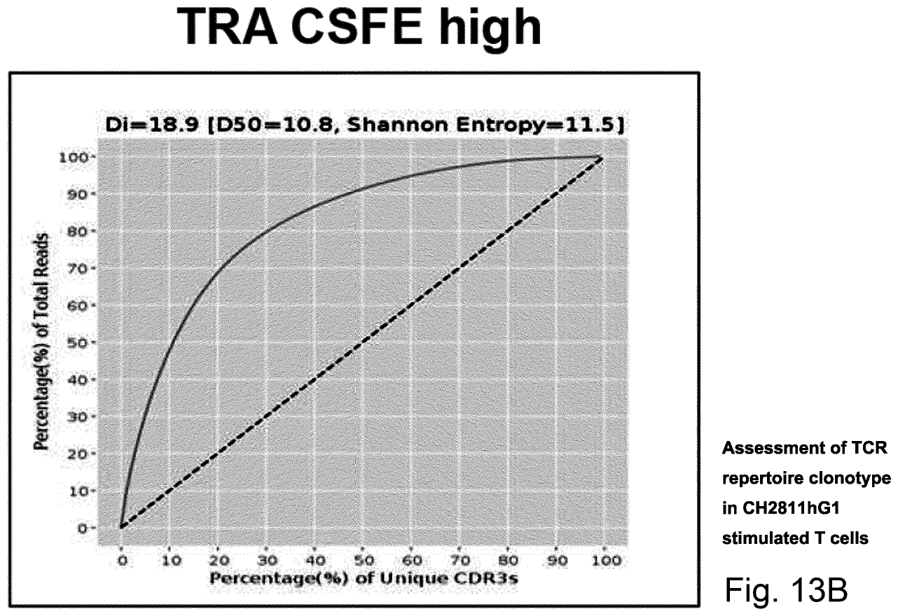

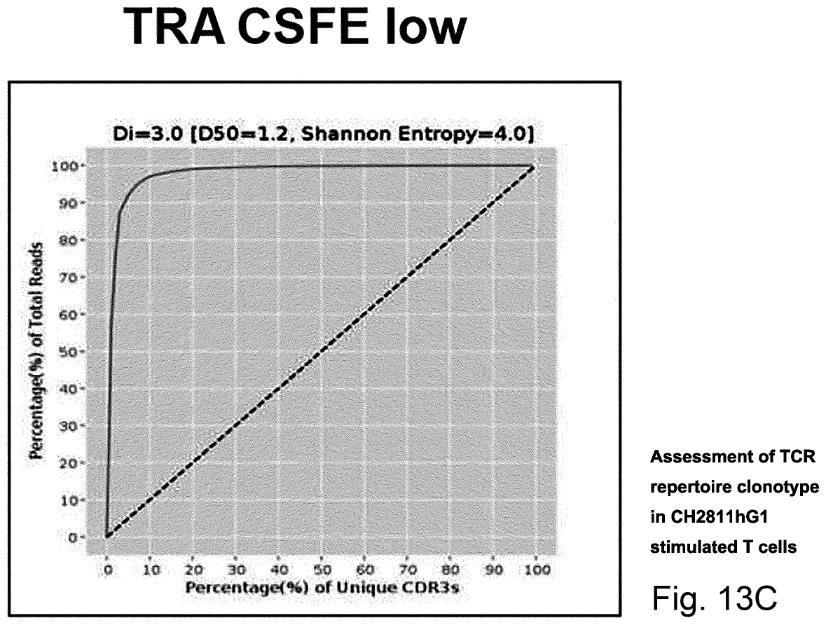

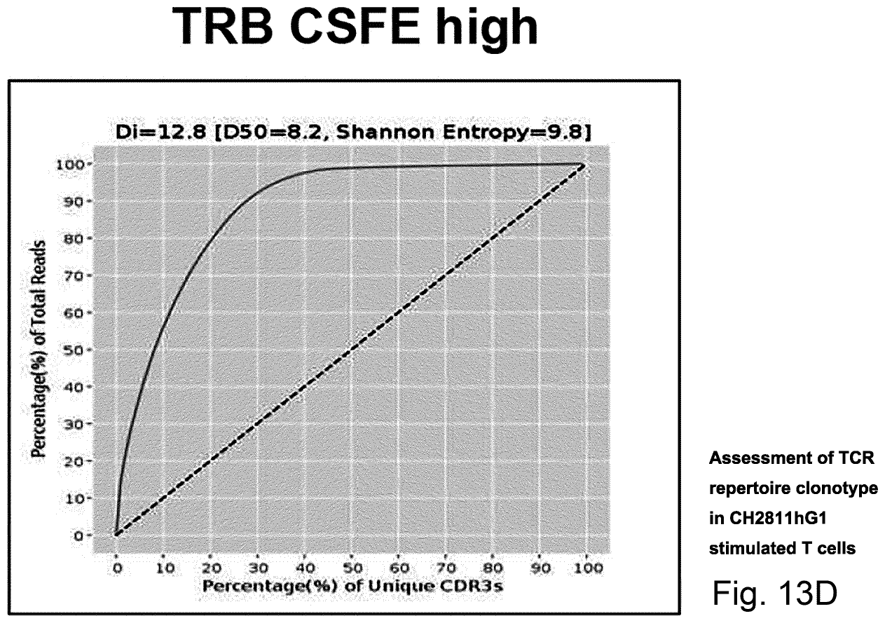

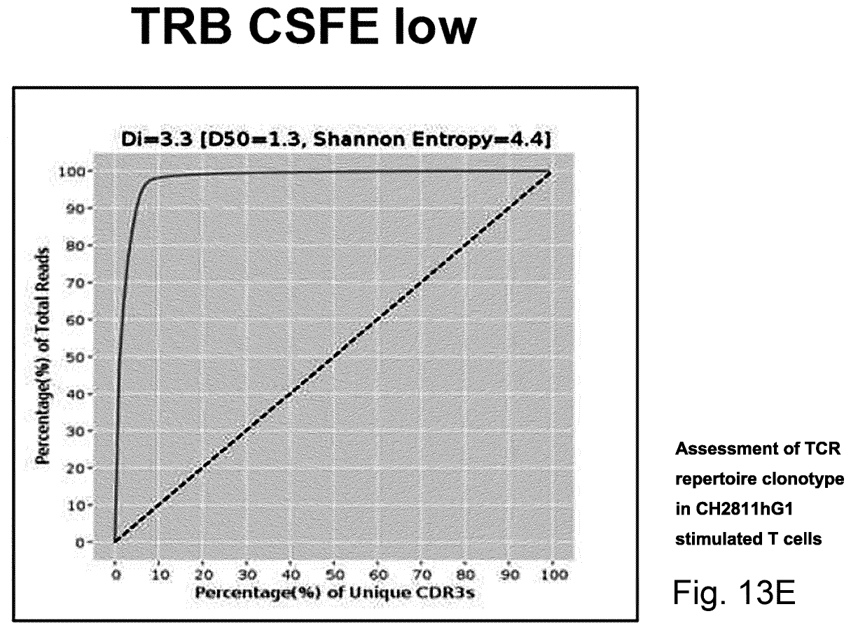

FIG. 13: Assessment of TCR repertoire clonotype in CH2811hG1 stimulated T-cells. T-cell repertoire is detected from the extracted RNA of CSFE high and low CH2811hG1 stimulated T-cells from 2 donors at day 19 (BD3) and 14 (BD26), respectively. TCR repertoire diversity is illustrated in tree maps where each rounded rectangular represents a unique entry: V-J-uCDR3 and the size of the spot denotes the relative frequency;
(A) Diversity plots for CH2811 stimulated T-cell CFSE high (B) and the CFSE low (C) TRA chain, CFSE high (D) and the CFSE low (E) TRB chain. The higher diversity of the sample, the closer the solid line is to the dashed line. The line assembles a curve that describes the overall diversity of the sample with “perfect” diversity being the black dashed line (each unique clonotype receives equivalent reads, i.e. no clonal expansion or dominant clone).

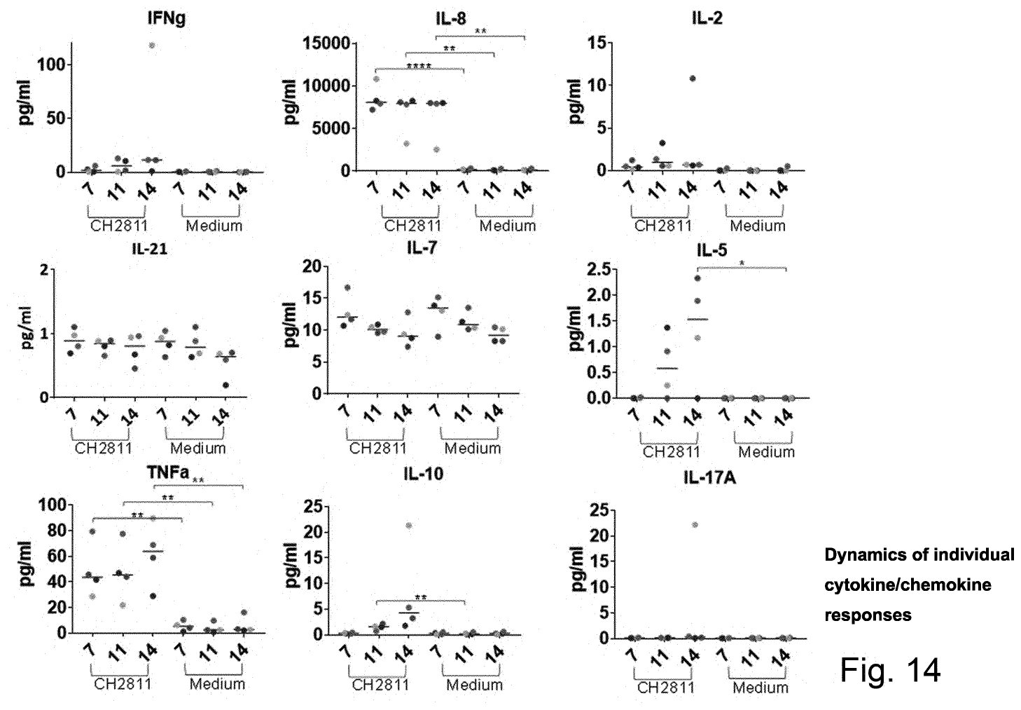

FIG. 14: Dynamics of individual cytokine/chemokine responses.

Pure T-cells isolated from 4 healthy donors (BD61, BD2, BD3, BD26) were stimulated with CH2811hG1 (5 μg/ml) at day 0. Unstimulated cells (medium) were included as negative control. Supernatants were collected at day 7, 11 and 14 and assessed for the concentration of IFNγ, TNFα, IL-8, IL-10, IL-2, IL-5, IL-17A, IL-7 and IL-21 (μg/ml). Individual dots represent different donors. Comparative analysis of the cytokine/chemokine results between CH2811hG1stimulated T-cells and unstimulated cells was performed by applying unpaired Student t test with values of P calculated accordingly (***, P<0.0001, **, P<0.01, *, P<0.05; GraphPad Prism 6).

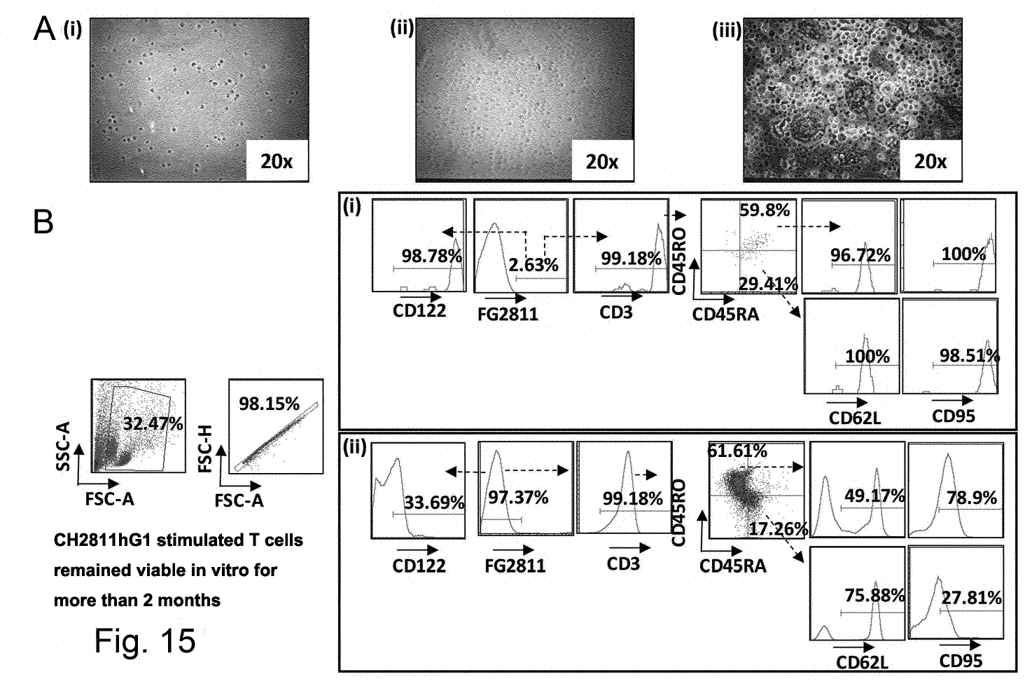

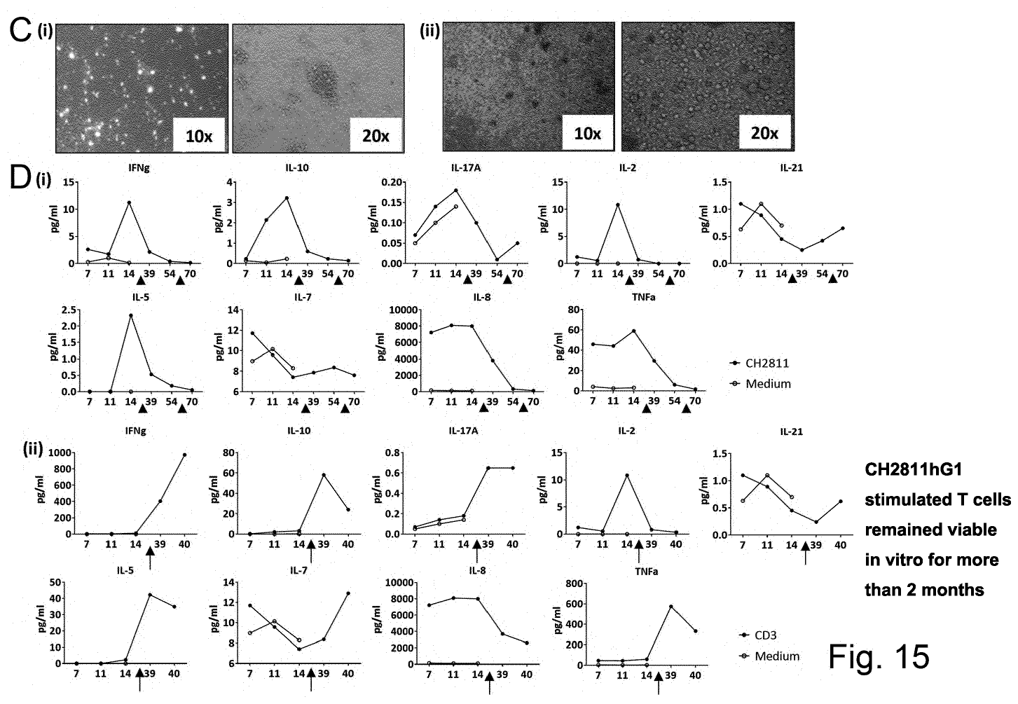

FIG. 15: CH2811hG1 stimulated T-cells remained viable in vitro for more than 2 months.
(A) T-cells were stimulated with plate bound CH2811hG1 (5 μg/ml) or anti-CD3 antibody (0.005 μg/ml) or medium at day 0. At day 35, under light microscope, cells stimulated with i) anti-CD3 antibody and ii) medium were all dead, except cells stimulated with iii) CH2811hG1 (magnification ×20);
(B) Phenotypic analysis of viable CH2811hG1 stimulated T-cells at day 35. Successive panels depicting the flow cytometric gating strategy used to phenotype FG2811mG3⁺ cells. Gates were drawn for analysis on i) FG2811mG3⁺ and ii) FG2811mG3⁻ cells; they were checked for CD3 and CD122 expressions. The CD3⁺ cells were further checked for the expression of CD45RA, CD45RO, CD62L and CD95 markers (result is representative of 1 donor);
(C) CH2811hG1 stimulated T-cells remained viable and maintained proliferative capacity at day 35 in vitro. At day 33, the viable CH2811hG1 stimulated T-cells were re-stimulated with plate bound CH2811hG1 (5 μg/ml) or the combination of plate bound anti-CD3 (0.005 μg/ml) and anti-CD28 (5 μg/ml) antibodies. Under the light microscope, the i) anti-CD3/CD28 re-stimulated T-cells underwent massive T-cell proliferation and formed T-cell blasts at day 39; ii) at day 70, CH2811hG1 stimulated cells remained viable and showed significant T-cell expansion (magnification ×10 and ×20);
(D) IL-7 and IL-21 could be crucial self-sustaining cytokines for the in vitro long-term survival of CH2811hG1 stimulated T-cells. Representative cytokine/chemokine expression levels (μg/ml) in i) CH2811hG1 and ii) anti-CD3/CD28 re-stimulated T-cells. T-cells were stimulated with CH2811 at day 0 followed by re-stimulation with either CH2811hG1 at day 33 and day 64 or with anti-CD3/CD28 antibodies at day 33. Supernatants were collected at day 7, 11, 14, 39, 54 and 70 and assessed for the concentration of IFNγ, IL-10, IL-17A, IL-2, IL-21, IL-5, IL-7, IL-8 and TNF-α (μg/ml). Triangles and arrows depicted 2811 and CD3/CD28 antibody re-stimulation day, respectively.

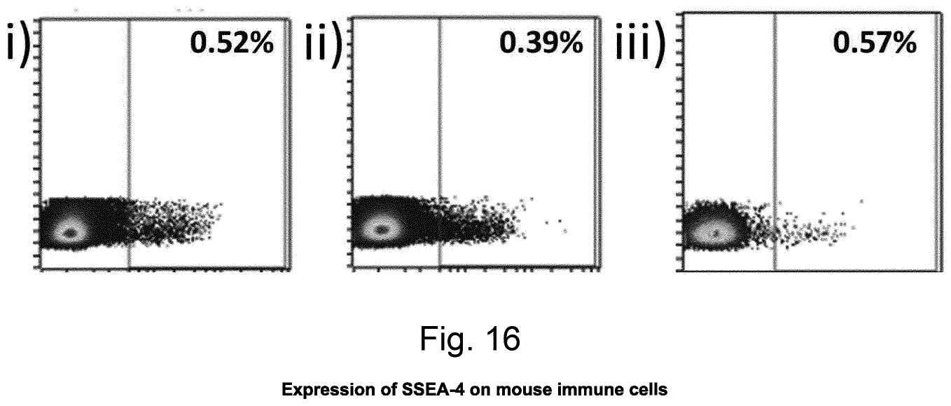

FIG. 16 Expression of SSEA-4 on mouse immune cells.

HHDII/DP4 mice were euthanised and spleen, mesenteric and inguinal lymph nodes were harvested. i) splenocytes, ii) mesenteric lymph node cells and iii) inguinal lymph node cells were stained with FITC-labelled CH2811hG1 antibody and assessed using flow cytometric analysis.

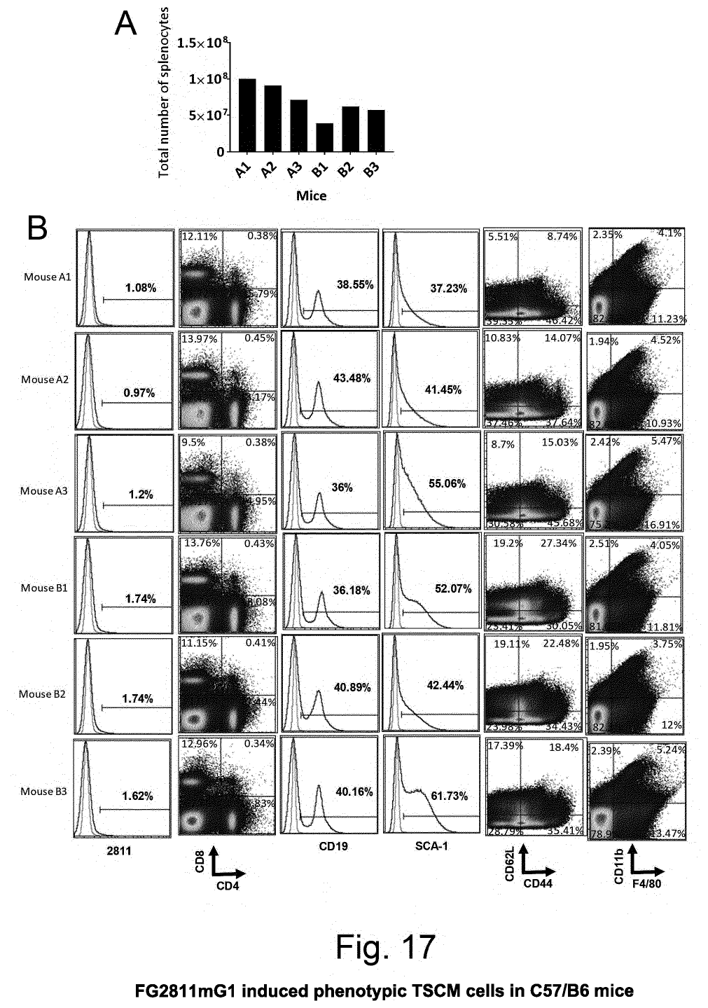

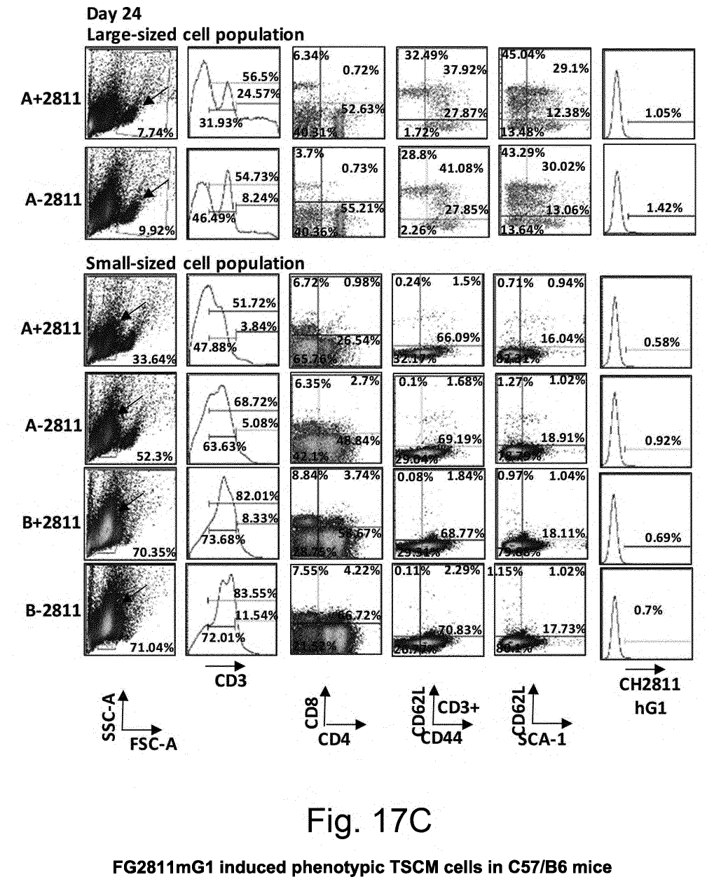

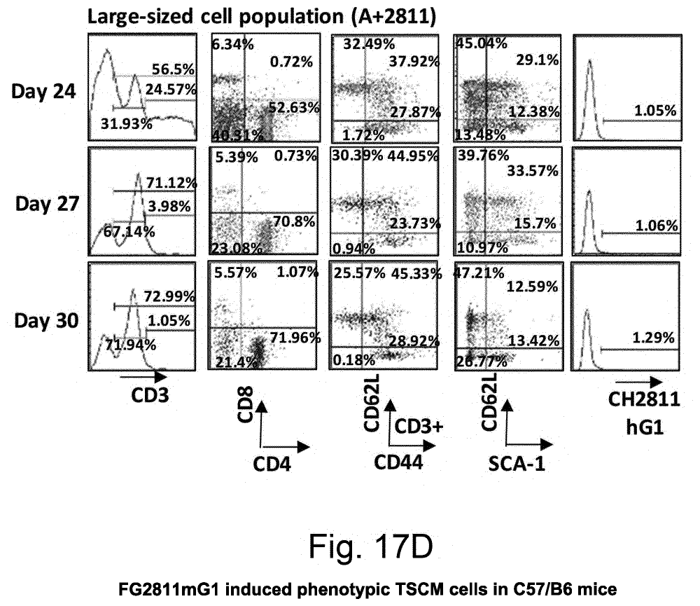

FIG. 17: FG2811mG1 induced phenotypic T_{SCM} cells in C57/B6 mice.
(A) At day 16, the total cell number of splenocytes from Group A and B were calculated using trypan blue exclusion analysis;
(B) At day 16, splenocytes from individual mouse from Group A and B were stained with CD4, CD8, CD44, CD62L, SCA-1 and CH2811hG1 antibodies and assessed using flow cytometric analysis;
(C) At day 16, Group A and Group B splenocytes were cultured with (A+2811 and B+2811) or without (A-2811 and B-2811) plate bound FG2811mG1 (5 μg/ml) and harvested at day 24. The day 24 splenocytes were stained with CD3, CD4, CD8, CD44, CD62L, SCA-1 and CH2811hG1 antibodies and assessed using flow cytometric analysis;
(D) At day 24, 27 and 30, A+2811 splenocytes were harvested and stained with anti CD3, CD4, CD8, CD44, CD62L, SCA-1 and CH2811hG1 and assessed using flow cytometric analysis.

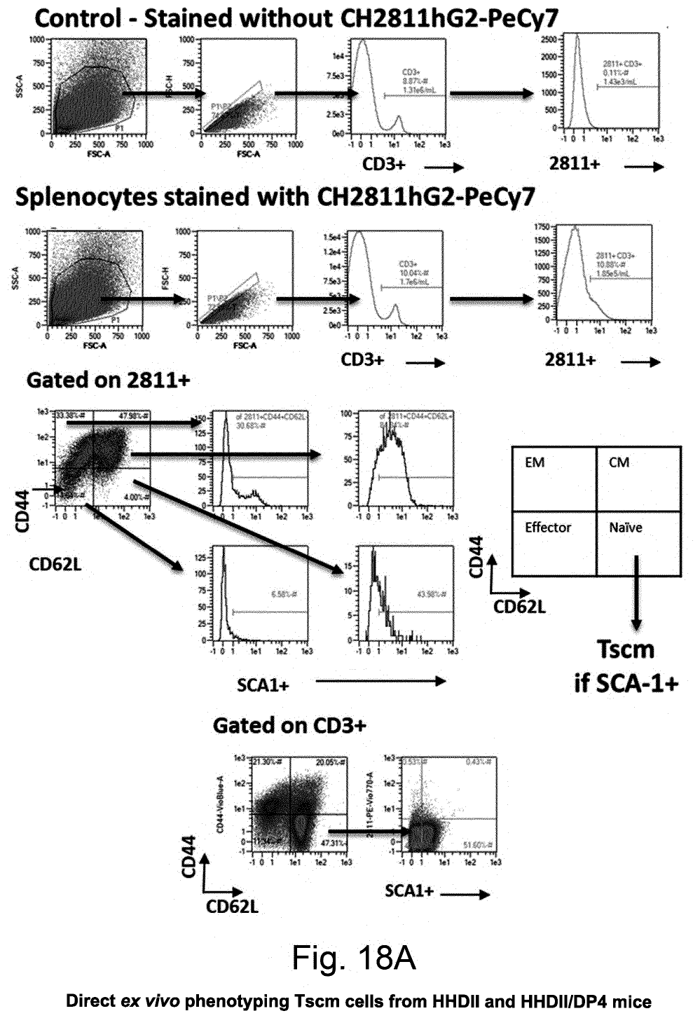

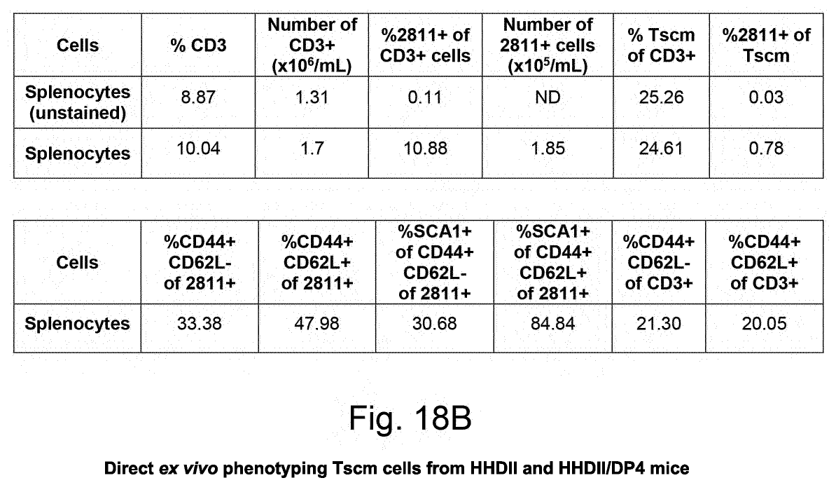

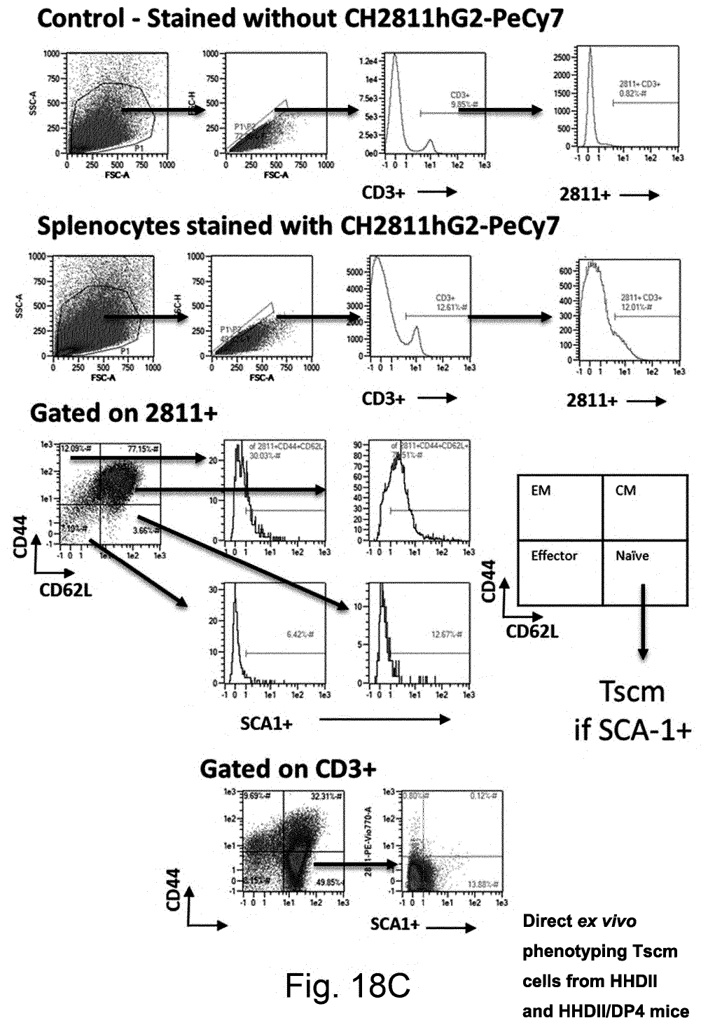

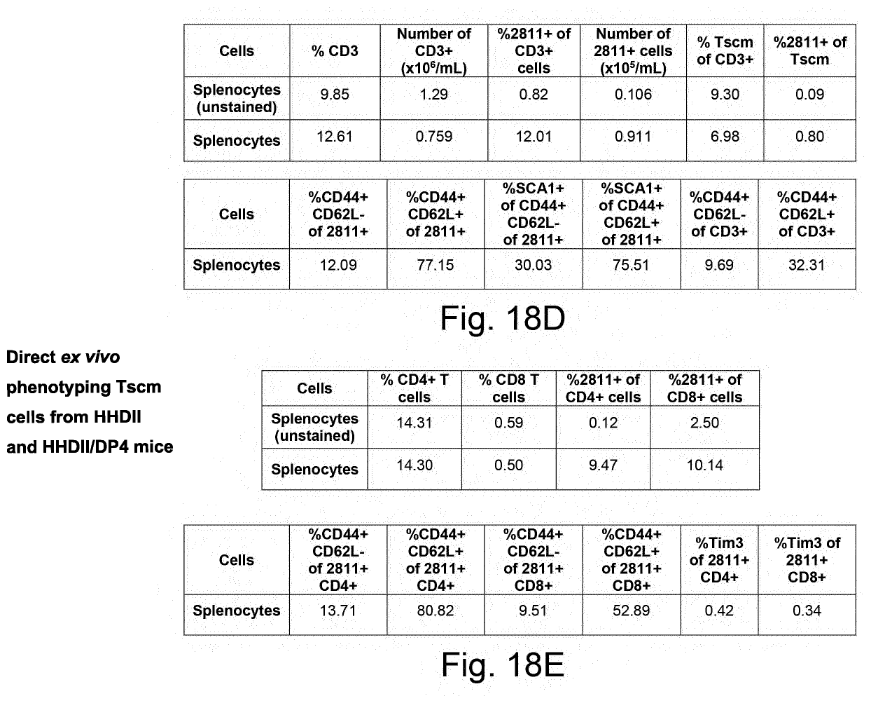

FIG. 18: Direct ex vivo phenotyping T_{SCM} cells from HHDII and HHDII/DP4 mice Naïve HHDII and HHDII/DP4 mice were culled, splenocytes were harvested and stained with CD3, CD44, CD62L, SCA-1 and CH2811hG2-PeCy7 antibodies and assessed using flow cytometry analysis.
(A) Representative flow cytometry plots of splenocytes stained direct ex vivo from HHDII mice.
(B) Summary of phenotyping results for splenocytes isolated from HHDII mice.
(C) Representative flow cytometry plots of splenocytes stained direct ex vivo from HHDII/DP4 mice.
(D) Summary of phenotyping results for splenocytes isolated from HHDII/DP4 mice.
(E) Summary of phenotyping results for CD4 and CD8 T cells isolated from HHDII/DP4 mice.

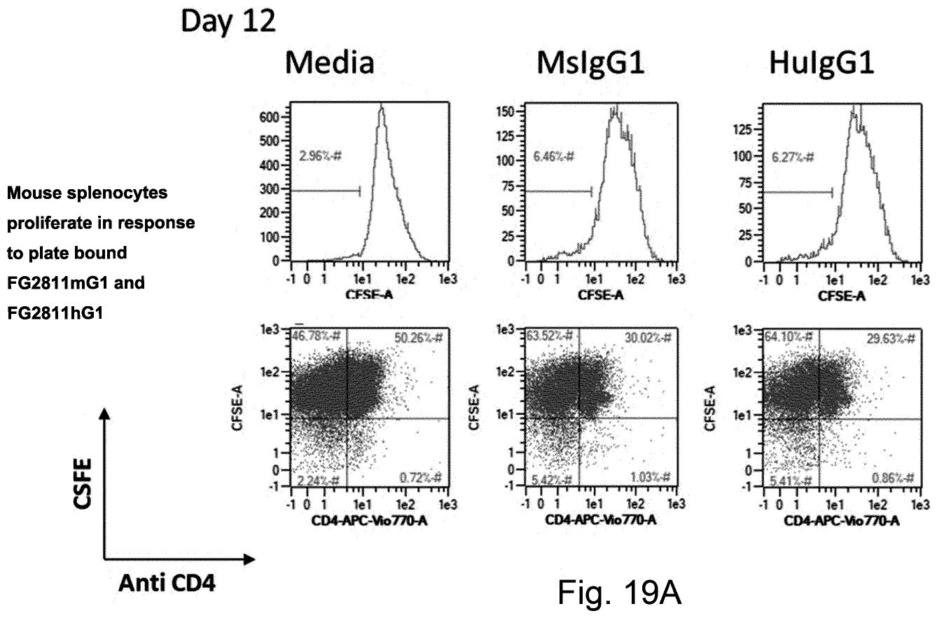

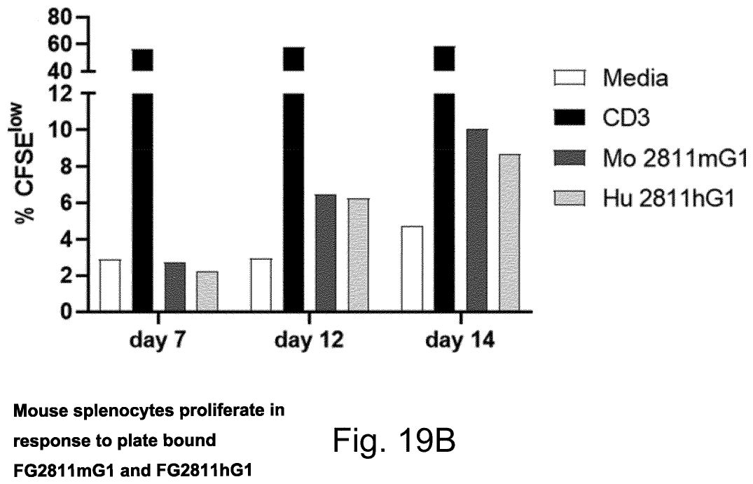

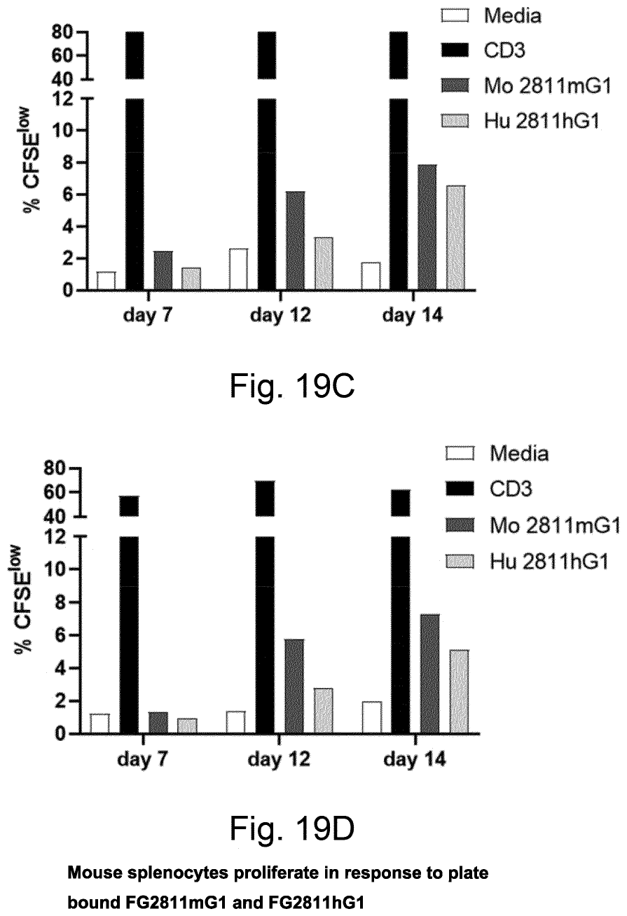

FIG. 19: Mouse splenocytes proliferate in response to plate bound FG2811mG1 and FG2811hG1

Naïve HHDII mice were culled, splenocytes were harvested, pan T cells enriched and CFSE labelled. CFSE labelled T cells were then plated out in wells contained plate bound 2811 mouse IgG1 (5 ug/mL) or Human IgG1 (5 ug/ml) or anti-CD3 (1 ug/ml) and incubated at 37° C. On day 7, 12 and 14, cells were taken as a sample and stained with anti CD4 and anti CD8 analysed by flow cytometry.
(A) At day 12, representative flow cytometry plot of T cells proliferating in response to FG2811mG1 and FG2811hG1.
(B) At day 12, the total percentage of cells proliferating in response to plate bound FG2811mG1, FG2811hG1 or anti CD3.
(C) At day 12, the total percentage of CD8 T cells proliferating in response to plate bound FG2811mG1, FG2811hG1 or anti CD3.
(D) At day 12, the total percentage of CD4 T cells proliferating in response to plate bound FG2811mG1, FG2811hG1 or anti CD3.

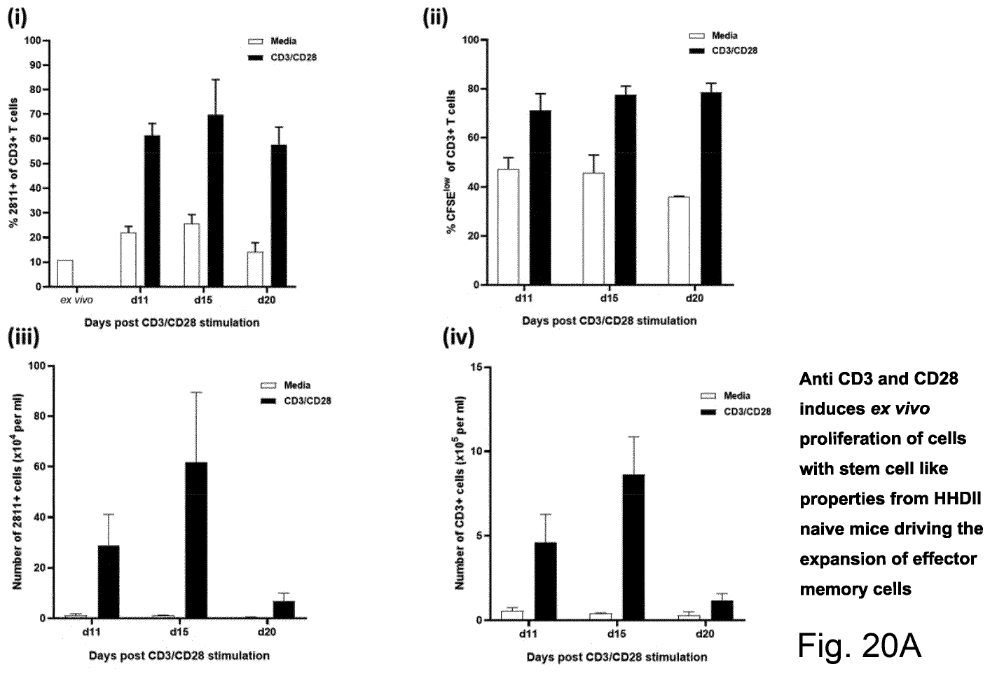

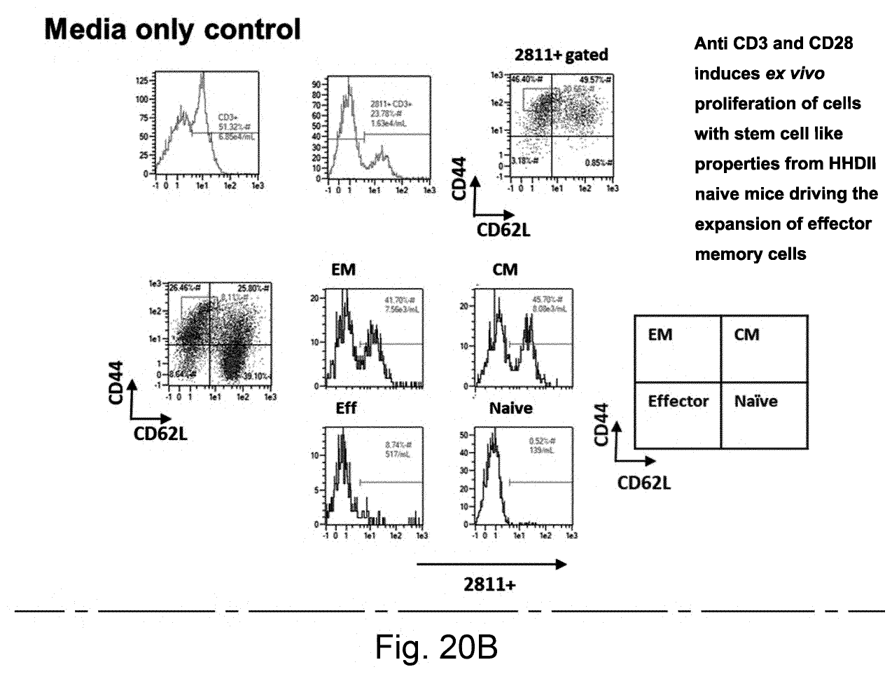

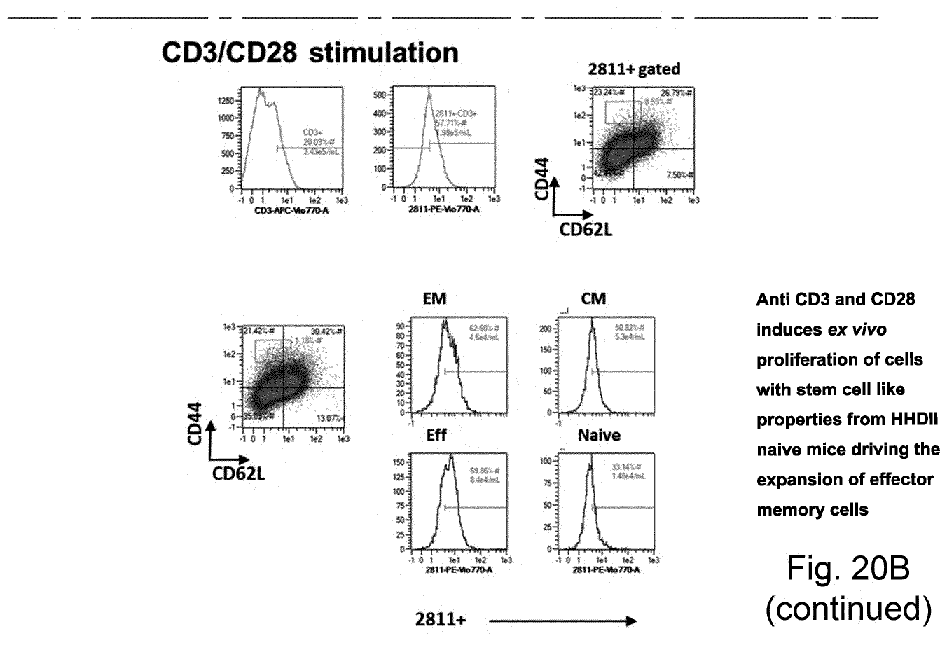

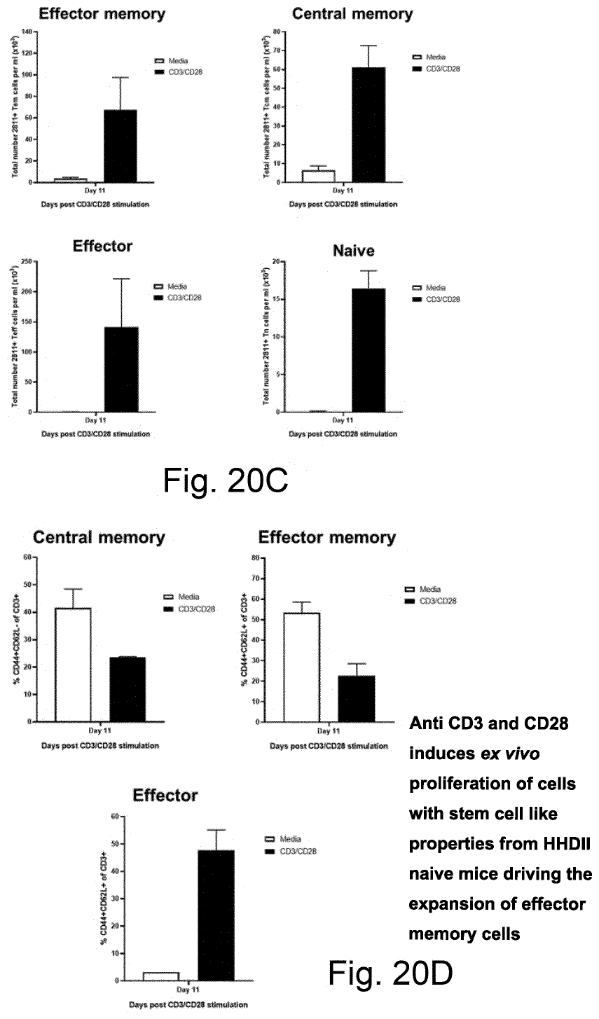

FIG. 20: Anti CD3 and CD28 induces ex vivo proliferation of cells with stem cell like properties from HHDII naive mice driving the expansion of effector memory cells

Naïve HHDII mice were culled, splenocytes were harvested, pan T cells enriched and CFSE labelled. CFSE labelled T cells were then plated out in wells containing anti CD3 and anti CD28 (1 ug/ml each) and incubated at 37° C. On day 7, 12 and 14, cells were taken as a sample and stained with anti CD4 and anti CD8 analysed by flow cytometry.
(A) At day 11, 15 and 20 cells were taken and stained with CH2811hG2-PeCy7 and/or anti CD3, (i) percentage 2811+ cells of CD3+ cells (ii) percentage CFSElow of CD3+ cells (iii) number of 2811+ cells (×10⁴ per mL) (iv) number of CD3+ T cells (×10⁵ per mL, (n=2 wells).
(B) Representative flow cytometry plots of splenocytes stained 11 days after CD3/CD28 stimulation, cells were stained with anti CD3, CD44, CD62L, and CH2811hG2-PeCy7 and assessed using flow cytometry analysis.
(C) After 11 days following CD3/CD28 stimulation the total number of 2811+ effector memory, central memory, effector and naive T cells was determined (n=2 wells).
(D) After 11 days following CD3/CD28 stimulation the percentage of 2811⁺ effector memory, central memory, effector and naive T cells was determined (n=2 wells).

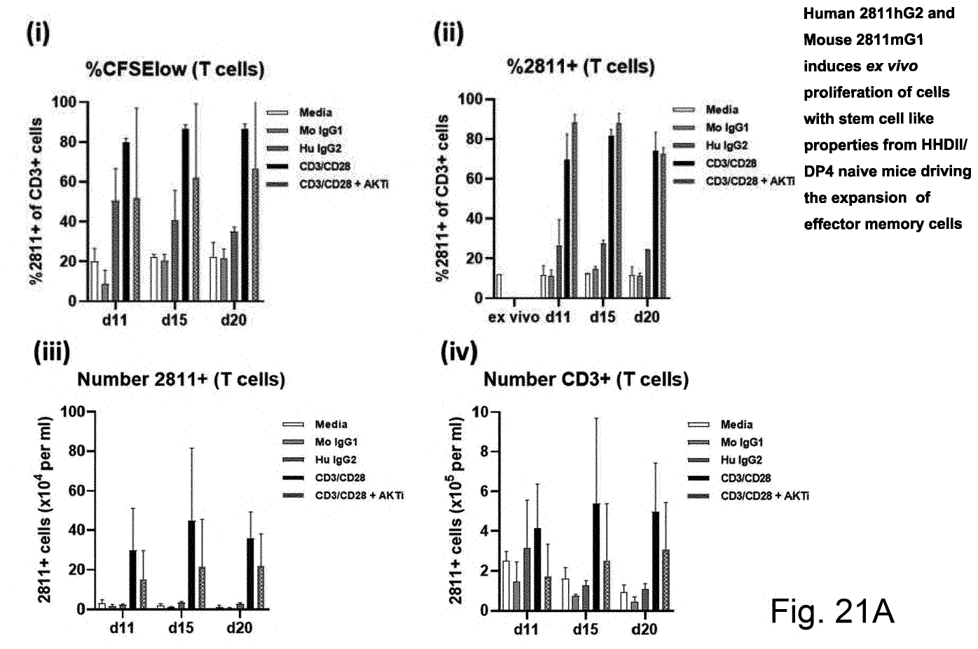

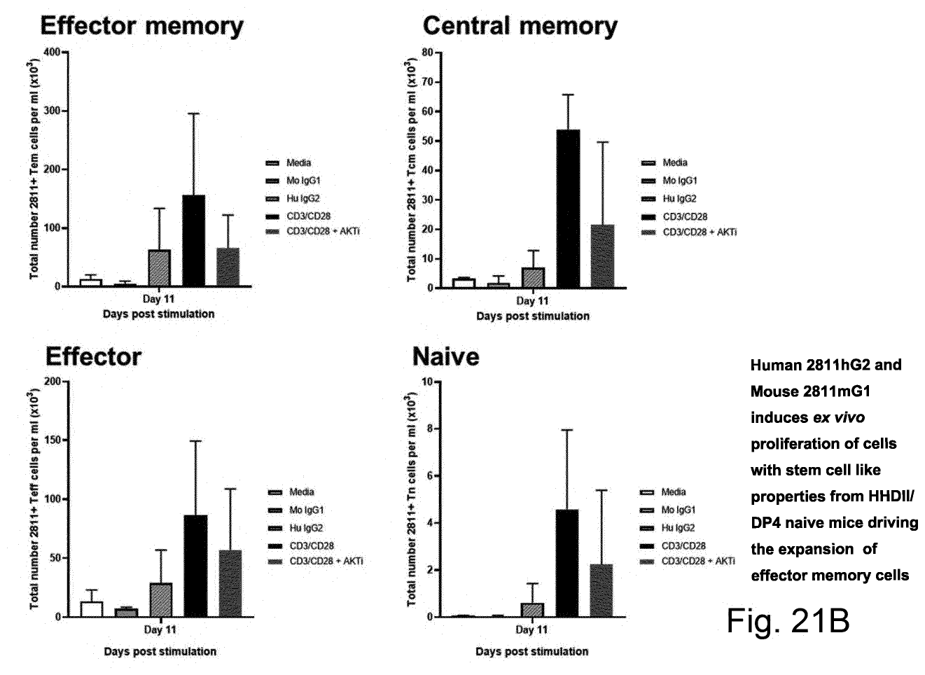

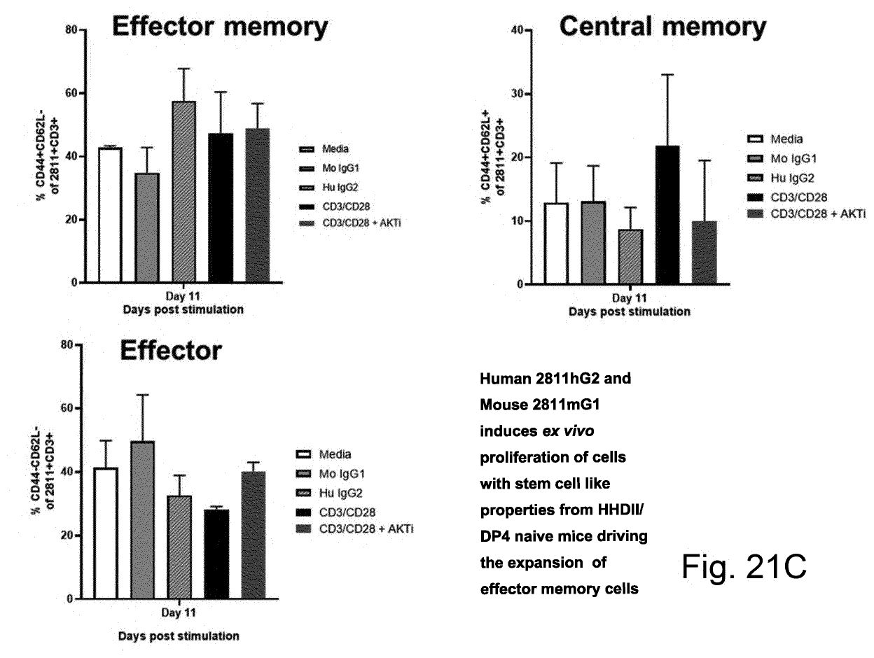

FIG. 21: Human 2811hG2 and Mouse 2811mG1 induces ex vivo proliferation of cells with stem cell like properties from HHDII/DP4 naive mice driving the expansion of effector memory cells

Naïve HHDII/DP4 mice were culled, splenocytes were harvested, pan T cells enriched and CFSE labelled. CFSE labelled T cells were then plated out in wells containing stimulation with soluble human IgG2 (5 ug/mL) or mouse IgG1 2811 Ab (5 ug/mL) or anti-CD3 (1 μg/ml) and CD28 with and without AKTi, cells were incubated at 37° C. On day 11 and 15, cells were taken as a sample and stained with anti CD3, CD44, CD62L, SCA 1 and CH2811hG2-PeCy7 and assessed using flow cytometry analysis.
(A) At day 11, representative flow cytometry plot of T cells proliferating in response to FG2811mG1 and 2811 hG2 (n=2 wells).
(B) After 11 days following CD3/CD28, FG2811mG1 or 2811 hG2 stimulation the total number of 2811⁺ effector memory, central memory, effector and naive T cells was determined (n=2 wells).
(C) After 11 days following CD3/CD28, FG2811mG1 or 2811 hG2 the percentage of 2811⁺ effector memory, central memory and effector T cells was determined (n=2 wells).

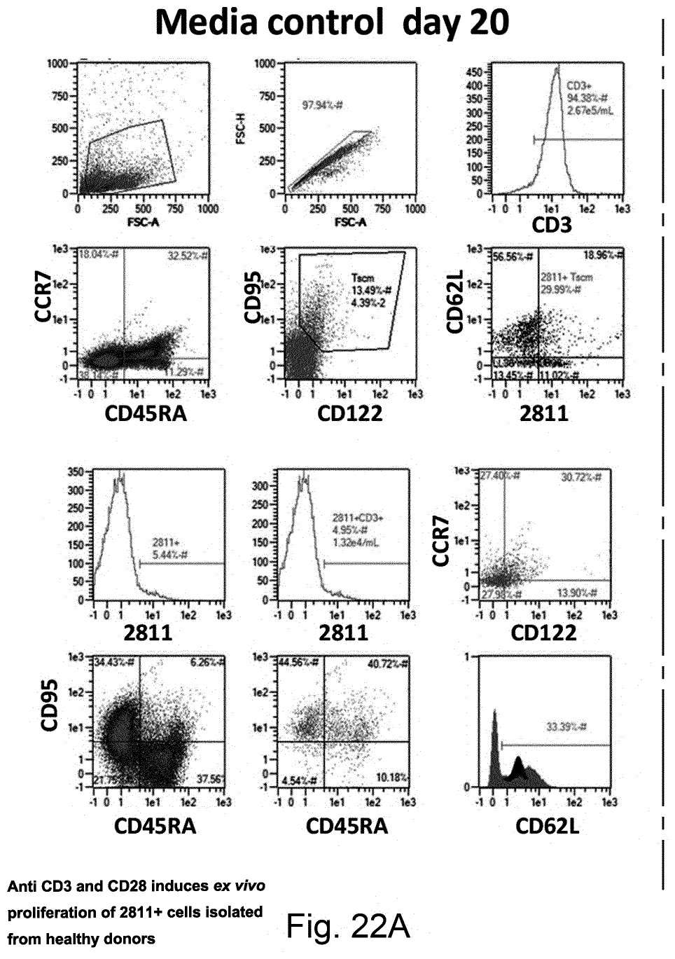

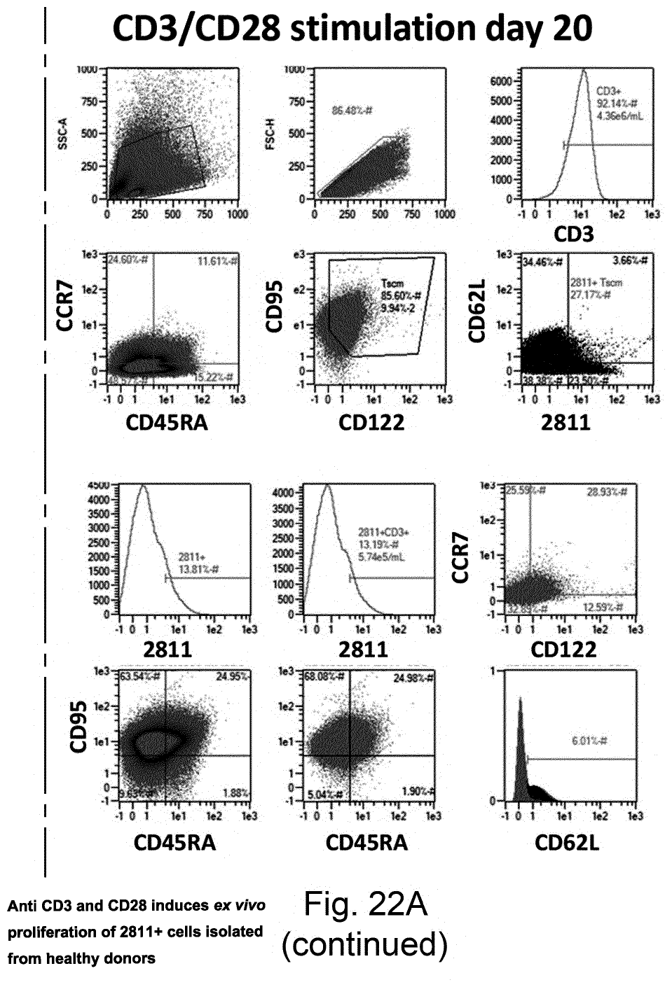

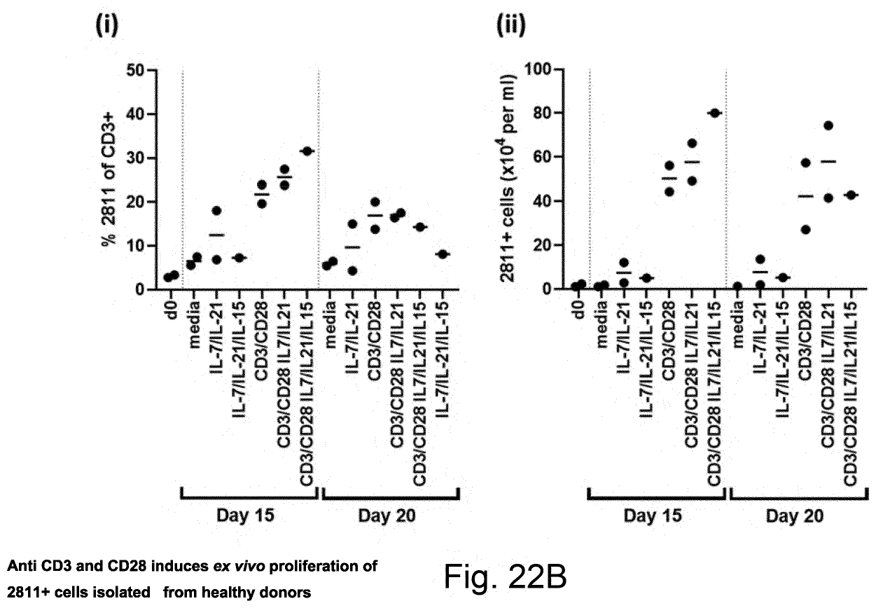

FIG. 22: Anti CD3 and CD28 induces ex vivo proliferation of 2811⁺ cells isolated from healthy donors

PBMCs were isolated from Buffy coats, a Pan T cell enrichment was carried out and approximately 2×10⁶ cells incubated per well of a 24 well plates, in the presence of anti CD3/CD28 with or without additional cytokines (IL-7 or IL-21) for 20 days. On day 15 and day 20 cells were taken as sample and stained with CD45RA, CD62L, CD122, CD95, CD3, CCR7 and 2811hG Pe-Cy7.
(A) At day 20, representative flow cytometry plot of phenotyping T cells proliferating in response to stimulation with anti CD3/CD28 (n=2 wells).
(B) After 15 and 20 days following CD3/CD28 stimulation the (i) percentage of 2811+ CD3+ T cells, (ii) total number of 2811+ cells (×10⁴ per mL, n=2 wells).

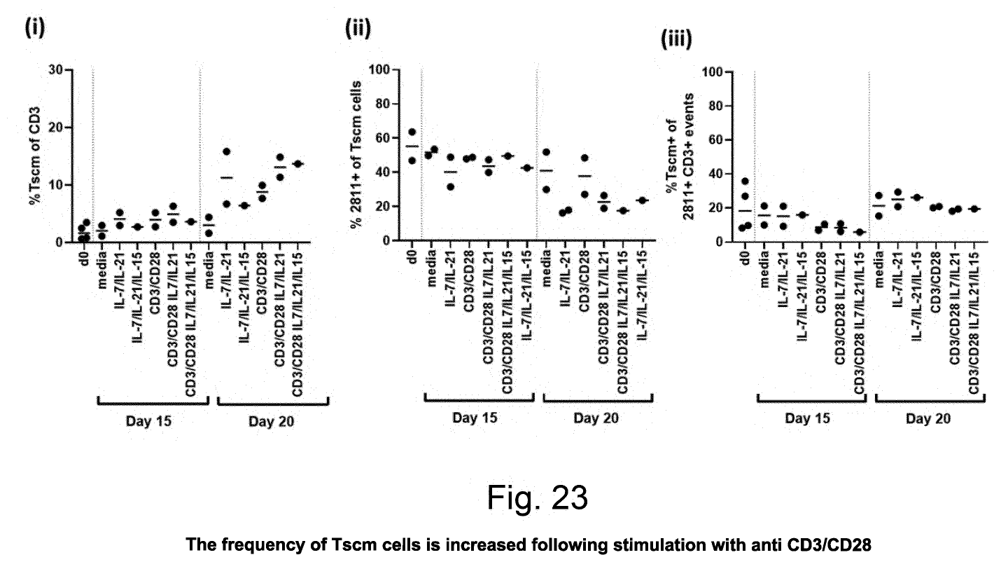

FIG. 23: The frequency of Tscm cells is increased following stimulation with anti CD3/CD28 PBMCs were isolated from Buffy coats, a Pan T cell enrichment was carried out and approximately 2×10⁶ cells incubated per well of a 24 well plates, in the presence of anti CD3/CD28 for 20 days. On day 15 and 20 cells were taken and Tscm staining according to the expression of CD3+CD45RA+CCR7+CD95+CD122^{low}. The percentage of Tscm cells in the CD3 T cell population (i), percentage of Tscm cells also 2811+(ii), the percentage of Tscm 2811+, CD3+ cells (iii).

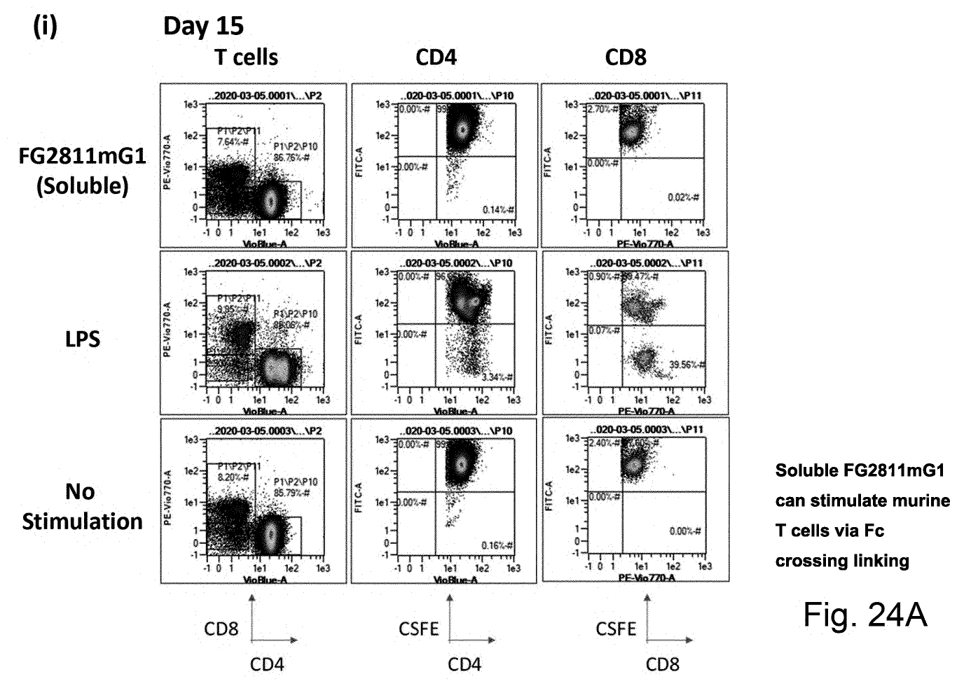

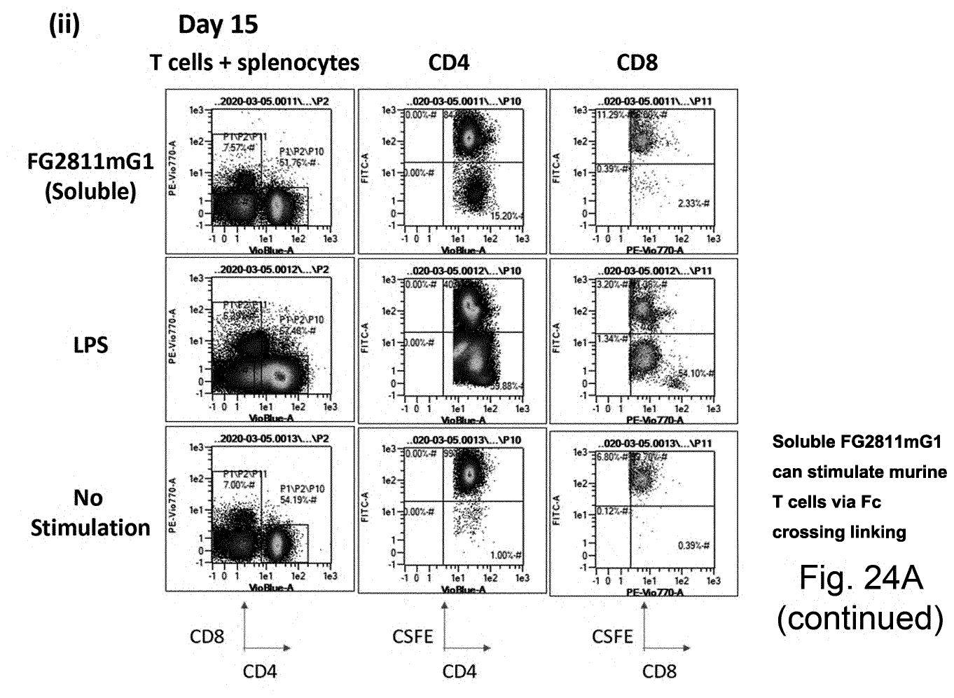

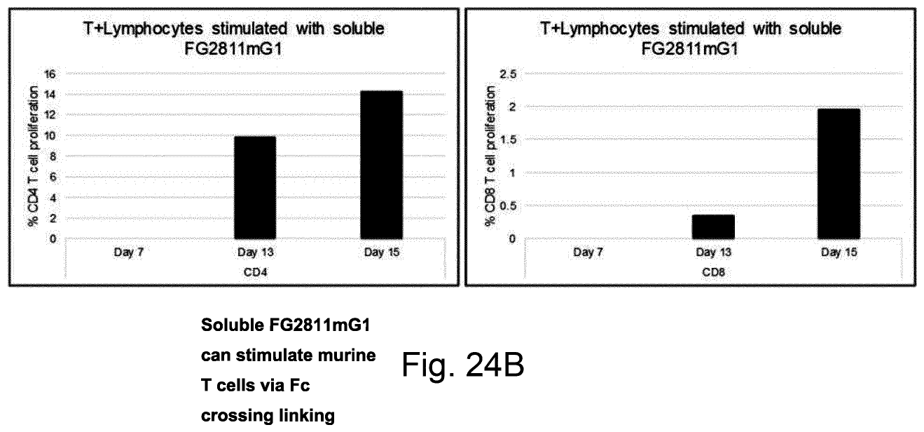

FIG. 24: Soluble FG2811mG1 can stimulate murine T cells via Fc crossing linking Splenocytes were isolated from HHDII and HHDII/FDP4 mice, pan T cells enriched from the splenocytes harvested from the HHDII mice. The HHDII pan T cells and HHDII/DP4 spenocytes were CFSE labelled. CFSE labelled T cells were either cultured alone or mixed at a 1:1 ratio with HHDII/DP4 splenocytes in the presence of soluble FG2811mG1, controls included media alone (negative) and LPS (positive control).
(A) (i) At day 15 a representative flow cytometry plot of T cells cultured in the pressence of FG2811mG1, LPS or media alone. The proliferating (CFSElow) and non proliferating (CFSEhigh) cells is shown for the CD4 and CD8 T cell populations.
   (ii) At day 15 a representative flow cytometry plot of T cells cultured with splenocytes in the pressence of FG2811mG1, LPS or media alone. The proliferating (CFSElow) and non proliferating (CFSEhigh) cells is shown for the CD4 and CD8 T cell populations.
(B) Summary of the proliferative responses of CD4 and CD8 T cells when cultured with or without splenocytes with FG2811mG1, LPS or media alone.

### DETAILED DESCRIPTION OF THE INVENTION

Methods

Plasma Membrane Glycolipid Extraction

SSEA-3/-4-LMTK cell pellets (5×10⁷ cells) were resuspended in 500 μl of Mannitol/HEPES buffer (50 mM Mannitol, 5 mM HEPES, pH7.2, both Sigma) and passed through 3 needles (23G, 25G, 27G) 30 times each. 5 μl of 1M CaCl₂ was added to the cells and passed through 3 needles 30 times each as above. Sheared cells were incubated on ice for 20 mins then spun at 3,000 g for 15 mins at room temperature. The supernatant was collected and spun at 48,000 g for 30 mins at 4° C. and the supernatant discarded. The pellet was resuspended in 1 ml methanol followed by 1 ml chloroform and incubated with rolling for 30 mins at room temperature. The sample was then spun at 1,200 g for 10 mins to remove precipitated protein. The supernatant, containing plasma membrane glycolipids, was collected and stored at −20° C.

Liposome Preparation

SSEA-3/-4-LMTK plasma membrane (μm) glycolipid extract (5×10⁷ cell equivalent) was mixed with a total concentration of 10 mgs of lipids [Cholesterol, dicetylphosphate (DCP),phosphatidylcholine (PC) and α-GalCer] in a round bottom flask at various ratios (Table 2). The lipid mixture was then dried down using a rotary evaporator at 60° C. until the solvent had evaporated, leaving a uniform lipid film on the wall of the flask. The flask was allowed to cool down to room temperature before the addition of 100 μl of sterile PBS. The opening of the flask was covered with parafilm and then immersed in an ultrasonic bath for 10 min to generate liposomes. (All work with chloroform and methanol was carried out in a fume hood).

Immunisation Protocol

BALB/c mice were between 6 to 8 weeks old (Charles River, UK). Prior to immunisation, normal mouse serum (NMS) was collected via tail bleed extraction, for use as a negative control, and stored at −20° C. Mice were immunised intraperitoneally (i.p.) with SSEA-3/-4-LMTK cells (1×10⁶ cells per immunization per mouse) at two weekly intervals using 1 ml insulin syringe (BD Bioscience, Spain). Seven days after the second immunisation, and every seven days for subsequent, anti-sera was collected via tail bleed extraction and screened for IgG and IgM antibody responses. Once a high titre of IgG response was obtained, the animal was boosted intravenously (i.v.) with SSEA-3/-4-LMTK cells (1×10⁵ cells per immunization per mouse) and sacrificed 5 days later.

mAb Generation

Isolation of splenocytes—Mice were euthanised and the spleen removed. After washing with 5 ml serum free medium (RPMI 1640) using a 25-gauge needle, the spleen was agitated with sterile forceps gently to harvest splenocytes. 5 ml of splenocytes were collected into a sterile 25 ml universal tube while excess fat and connective tissues was discarded. The total fluid volume containing the splenocytes was increased to 25 ml with serum free medium (RPMI 1640) and centrifuged at 100 g for 10 mins. The supernatant was removed, leaving 1 ml of medium and the splenocytes which were then resuspended in 5 ml serum free medium (RPMI 1640) and counted using a haemocytometer with trypan blue, staining for viability assessment.

Fusion of splenocytes with NS0 myeloma cells—Washed splenocytes were combined with healthy NS0 myeloma cells in a ratio of 1:10 (NS0: splenocytes; 1×10⁷: 1×10⁸ cells) in a 25 ml universal tube and centrifuged at 317 g for 5 mins. The supernatant was aspirated and the combined cell pellet was resuspended in 800 μl of polyethylene glycol (PEG) gently and gradually over 1 min. The cell mixture was agitated gently for 1 min prior to the addition of 1 ml of serum free medium (RPMI 1640) over 1 min while continuing to agitate. A further 20 ml of serum free medium (RPMI 1640) was added over 1 min while continuing to agitate. Then the cell mixture was centrifuged at 317 g for 5 mins, the supernatant removed and the cell mixture was resuspended in 15 ml of hybridoma medium [500 ml hybridoma serum free medium (Gibco): 10 ml HT (hypoxanthine thymidine) supplement (50× Hybri-Max; Sigma): 31 μl (31 μg) methotrexate (1 mg/ml; Sigma): 25 ml of Hybridoma cloning factor (Opti-Clone 11; MP): 50 ml of filtered NS0 spent medium]. The cell suspension was spread evenly across a 96 well flat bottom plate and incubated at 37° C. in cell culture incubator (5% CO2).

CH2811 hG1 Generation

Total RNA was prepared from 5×10⁶ FG2811 hybridoma cells using Trizol (Invitrogen, Paisley, UK), following the manufacturer's protocol. First-strand cDNA was prepared from 3 μg of total RNA using a first-strand cDNA synthesis kit and AMV reverse transcriptase following the manufacturer's protocol (Roche Diagnostic). PCR and sequencing of heavy and light chain variable regions was performed by Syd Labs, Inc (Natick, Mass. 01760, USA) and variable region family usage analysed using the IMGT database (Lefranc et al. 2018). FG2811 variable regions were subsequently cloned into the hIgG1/kappa double expression vector pDCOrig-hIgG1 (Metheringham et al. 2009) and the sequence confirmed by sequencing.

mAb Characterisation

mAb isotyping—Spent hybridoma serum free medium (Invitrogen Scotland, UK) was collected and 150 μl diluted in 1/10 dilution in PBS 1% (w/v) BSA and then pipetted into the development tube of the mouse mAb isotyping test kit (AbD Serotec, Kidlington, UK) and incubated at room temperature for 30 seconds. The tube was vortexed briefly to ensure the coloured microparticle solution was completely resuspended. One isotyping strip was placed into the tube, with the solid red end of the strip at the bottom of the tube for 5 to 10 mins. The result was interpreted by checking the blue bands appeared above the letters in one of the class or subclass windows as well as either kappa or lambda window of the strip, indicating the heavy and light chain composition of the mAb.

Mouse mAb purification— 2 litres of spent hybridoma serum free medium (Invitrogen Scotland, UK) was collected and 0.2% sodium azide (Sigma) added. The spent medium was subsequently filtered through Whatman paper followed by filtration using 0.2 μm steritop filters (Sigma). A HiTrap Protein G HP antibody purification column (GE Healthcare) was used for the purification according to the manufacturer's recommendations. mAb binding buffer consisted of PBS-Tris pH 7.0 and mAb was eluted using Tris-Glycine pH12.0. Fractions containing IgG mAb were pooled, pH-neutralised using 10M HCl and dialysed overnight against PBS, before aliquoting and storing at −80° C.

Transient mAb production—The FG2811mG1, CH2811hG1 and CH2811hG2 mAbs were obtained following transient transfection of Expi293TM cells using the ExpiFectamine™293 Transfection kit (Gibco, Life Technologies). The HEK293 cells in suspension (100 ml, 2×10⁶ cells/ml) were transfected with 100 μg plasmid DNA and conditioned medium harvested at day seven, post transfection.

Tumour Cell Lines

Cell lines were maintained by regular replacement of complete culture media and splitting to maintain log phase growth. All cell lines were regularly checked for mycoplasma contamination and authenticated using short tandem repeat (STR) profiling (Table 1).

TABLE 1
Cancer cell lines.

| | Source | Cancer type | Media |
|---|---|---|---|
| Human cancer | | | |
| cell line | | | |
| U251MG | ATCC | GBM (adult) | EMEM 10% FCS |
| U87MG | ATCC | GBM (adult) | EMEM 10% FCS |
| SF188 | ATCC | GBM (adult) | EMEM 10% FCS |
| KNS42 | ATCC | GBM (paediatric) | EMEM 10% FCS |
| UW2283 | ATCC | Medulla blastoma | EMEM 10% FCS |
| DAOY | ATCC | Medulla blastoma | EMEM 10% FCS |
| T47D | ATCC | Ductal carcinoma | RPMI 10% FCS |
| DU4475 | ATCC | Breast carcinoma | RPMI 10% FCS |
| MCF7 | ATCC | Breast adenocarcinoma | RPMI 10% FCS |
| Colo205 | ATCC | Colorectal adenocarcinoma | RPMI 10% FCS |
| | | (Duke D) | |
| OVCAR3 | ATCC | Ovarian adenocarcinoma | RPMI 10% FCS |
| OVCAR5 | ATCC | Ovarian adenocarcinoma | RPMI 10% FCS |
| IGROV1 | ATCC | Ovarian adenocarcinoma | RPMI 10% FCS |
| Mouse fibroblast | | | |
| cell line | | | |
| LMTK | ATCC | Mouse fibroblast | DMEM 10% FCS |
| SSEA-3/-4-LMTK | IJC Josep Carreras | SSEA-3/-4-expressing LMTK | DMEM 10% FCS |
| | Leukemia Research | mouse fibroblast cells | |
| | Institute | | |
| NS0 | ATCC | Myeloma cells | RPMI 10% FCS |

GBM: Glioblastoma Multiforme

Antibody Binding to Cancer Cell Lines and Mouse Fibroblast Cells.

1×10⁵ cells were incubated with 50 μl of primary antibodies (of various concentrations) at 4° C. for 1 hr. Cells were washed with 200 μl of RPMI 10% FCS and spun at 100 g for 5 mins. Supernatant was discarded and 50 μl of FITC-conjugated anti-mouse/anti-human or biotin-conjugated anti-mouse/anti-human IgG/IgM Fc specific antibody (Sigma) diluted 1/100 in RPMI 10% FCS was used as secondary antibody. Cells were incubated in dark for 1 hr at 4° C. Cells were washed with 200 μl of RPMI 10% FCS and spun at 100 g for 5 mins. 50 μl of streptavidin-FITC (Sigma) or Strep-PeCy7 (eBioscience) diluted 1/100 in RPMI 10% FCS were used to detect biotinylated secondary antibody. Cells were washed with 200 μl of RPMI 10% FCS and spun at 100 g for 5 mins.

Cells were fixed with 0.4% formaldehyde and analysed on Beckman Coulter Fc-500 flow cytometer (Beckman Coulter, High Wycombe, UK) or MACSQ flow cytometer (Miltenyi Biotech, Bisley, UK).

Antibody Binding to Whole Blood

50 μl of healthy donor whole blood was incubated with 50 μl primary antibody at 4° C. for 1 hr. The blood was washed with 150 μl of RPMI 10% NBCS and spun at 100 g for 5 mins. Supernatant was discarded and 50 μl of FITC conjugated anti-mouse/anti-human or biotin-conjugated anti-mouse/anti-human IgG Fc specific antibody (Sigma; 1/100 in RPMI 10% NBCS) was used as secondary antibody. Cells were incubated at 4° C. in the dark for 1 hr then washed with 150 μl RPMI 10% NBCS and spun at 100 g for 5 mins. 50 μl of streptavidin-FITC (Sigma; 1/100 in RPMI 10% NBCS) or streptavidin-PE-Cy7 (eBioscience; 1/100 in RPMI 10% NBCS) was used to detect biotinylated secondary antibody. Cells were incubated at 4° C. in dark for 1 hr then washed with 200 μl RPMI 10% NBCS and spun at 100 g for 5 mins. After discarding the supernatant, 50 μl/well Cal-Lyse (Invitrogen, Paisley, UK) was used followed by 500 μl/well distilled water to lyse red blood cells. The blood was subsequently spun at 100 g for 5 mins, the supernatant discarded and the cells were resuspended in 500 μl PBS. Samples were analysed on a FC-500 flow cytometer (Beckman Coulter). To analyse and plot raw data, WinMDI 2.9 software was used.

TLC Analysis of Glycolipid Binding

LMTK and SSEA-3/-4-LMTK plasma membrane lipid samples were blotted onto silica plates and developed in chloroform (Sigma)/methanol (Sigma)/distilled water (60:30:5 by volume) twice followed by hexane (Sigma):diethyl ether (Sigma):acetic acid (Sigma) (80:20:1.5 by volume) twice. The dried plates were sprayed with 0.1% polyisobutylmethacrylate (Sigma) in acetone. After air drying, the plates were blocked with PBS 2% (w/v) BSA for 1 hr at room temperature and incubated overnight at 4° C. with primary antibodies diluted in PBS 2% (w/v) BSA. The plates were then washed 3 times with PBS and incubated with biotin—conjugated anti-mouse IgG Fc specific secondary antibody (Sigma) diluted 1/1000 in PBS 2% (w/v) BSA for 1 hr at room temperature. The plates were subsequently washed again in PBS before incubating with IRDye 800CW streptavidin (LICOR Biosciences, Cambridge, UK) diluted 1/1000 in PBS 2% (w/v) BSA for 1 hr at room temperature in the dark. The plates were subsequently washed a further 3 times with PBS and air dried in the dark. Lipid bands were visualized using a LICOR Odyssey scanner.

Glycan (Coupled to HSA) ELISA

ELISA plates (Becton Dickinson, Oxford, UK) were coated overnight at 4° C. with 100 ng/well of SSEA-3, SSEA-4, Forssman, Globo-H and Sialyl-Lewis x (SLex) glycan-HSA conjugates, resuspended in PBS (Elicityl, Crolles, France), blocked with 200 μl/well of PBS 5% (w/v) BSA for 1 hr at room temperature, followed by incubation with 50 μl/well of primary antibodies (5 μg/ml). The primary antibodies were detected using biotinylated anti-mouse IgG or anti-rat IgM secondary antibody (Sigma) diluted 1/5000 in PBS 1% (w/v) BSA. After incubation with streptavidin horseradish peroxidase (HRPO) conjugate (Invitrogen) diluted 1/5000 in PBS 1% (w/v) BSA and development with 3,3′,5,5′-Tetramethylbenzidine (TMB; Sigma), plates were read at 450 nm using Tecan Infinite F50.

Erythrocyte Binding Assay

Healthy donor erythrocytes were washed thrice in PBS and resuspended in 10 times the packed cell volume of PBS. 50 μl of washed erythrocytes were then incubated with 50 μl of primary antibodies at 37° C. for 1 hr. Cells were washed with 150 μl of PBS and spun at 100 g for 5 mins. Supernatant was discarded and cells resuspended in 50 μl FITC-conjugated anti-mouse IgG Fc specific secondary antibody (Sigma) diluted 1/100 in PBS 1% (w/v) BSA. Cells were incubated at 37° C. in the dark for 1 hr then washed with 150 μl PBS and spun at 100 g for 5 mins. Supernatant was discarded and cells were resuspended in 500 μl PBS. Samples were analysed by FC-500 flow cytometer (Beckman Coulter). To analyse and plot raw data, WinMDI 2.9 software was used.

Erythrocyte Hemagglutination Assay

4 ml of normal donor whole blood was collected into a heparin tube (Becton Dickinson). The whole blood was transferred to a sterile 15 ml conical tube and washed with sterile PBS. The washed blood was centrifuged at 100 g for 5 mins. The supernatant was aspirated. The washing step was repeated twice. After the final wash, the blood cell pellet was diluted with sterile PBS to make a final working concentration of 0.5% erythrocytes. 50 μl of 0.5% erythrocytes was added into each well of a 96 well U bottom plate. On top of erythrocytes, primary antibodies were added at 50 μl/well and incubated at room temperature for 1 hr or until the erythrocytes agglutinated.

Monoclonal Antibody Affinity Analysis

The kinetic parameters of the FG2811mG3 mAb binding to SSEA-4-containing liposomes was determined by Surface Plasmon Resonance (SPR, Biacore 3000, GE Healthcare). An L1 sensor chip (GE-healthcare) was preconditioned with 40 mM octyl D-glucoside, followed by coating with SSEA4-containing liposomes (6000RU) and a short pulse of NaOH (10 mM) to remove loosely bound liposomes. The reference flow cell was treated in the same manner with the exception that liposomes devoid of SSEA-4 were used. For both flow cells the degree of coverage was near complete as injection of HSA (0.1 mg/ml) induced a marginal increase in RU (50-60 RU). After stabilisation of the signal from both flow cells, increasing concentrations (0.3 nmol/L-200 nmol/L) of the FG2811mG3 mAb were injected, followed by regeneration (10 mM glycine pH 1.5) after cycle. Binding curves were fitted to a 1:1 (Langmuir) binding model using BIAevaluation 4.1.

Glycome Analysis of FG2811mG3 (Consortium for Functional Glycomics)

To determine the fine specificities of the FG2811mG3 antibody, the antibody was FITC-labelled and sent to the Consortium for Functional Glycomics where they were screened against 600 natural and synthetic glycans (core H group, version 5.1). The synthetic and mammalian glycans with amino linkers were printed onto N-hydroxysuccinimide (NHS)-activated glass microscope slides, forming amide linkages. Printed slides were incubated with 5 μg/ml of antibody for 1 hr at room temperature before the binding was detected with Alexa488-conjugated goat anti-mouse IgG. Slides were then dried, scanned and the screening data compared to the Consortium for Functional Glycomics database.

CSFE T-Cell Proliferation Assay

PBMC Separation

Whole blood (buffy coats) were obtained from the national blood service (Sheffield) or was collected from healthy donors in a syringe containing lithium heparin (1000 units/ml; Sigma H0878). Whole blood was diluted with RPMI 1640 at 1:1 ratio and layered on lymphocyte separation medium (Histopaque-1077; Sigma), followed by centrifugation at 800 g, off brake for 25 mins. After centrifugation, plasma was collected from top layer, PBMCs from the buffy coat layer. PBMCs were washed with RPMI 1640 twice and spun at 317 g for 5 mins. Number of PBMCs was counted and the cells were ready for T-cell isolation.

Pure T-Cell Isolation

Every 1×10⁷ PBMCs was resuspended in 40 μl of cold MACS buffer [PBS 1% (v/v) FCS 1% (v/v) EDTA] (PBS: Sigma D8537; FCS: Sigma F9665 and 0.5M pH8 EDTA: Invitrogen). Then 10 μl of Pan T cell Biotin antibody (Miltenyi) was added to every 1×10⁷ cells and incubated at 4° C., in dark for 5 mins. 30 μl of cold MACS buffer was added to every 1×10⁷ cells followed by 20 μl of Pan T cell microbead (Miltenyi) to every 1×10⁷ cells and incubated at 4° C., in dark for 10 mins. Cells were added to LS column (Miltenyi) and the flow through, which contained the CD3 purified T cells were collected. Cells retained in the column were non T cells.

CSFE Loading

Purified T cells were washed with RPMI 1640 and the number of cells were counted. Cells were spun at 317 g for 5 mins and supernatant was removed. Every 1×10⁷ purified T cells was resuspended in 1 ml of PBS 10% FCS. CSFE was dissolved in 18 μl DMSO (Invitrogen) followed by 1.8 ml of PBS 10% FCS. Then, 110 μl of diluted CSFE was added to every 1×10⁷ T cells and incubated in dark at room temperature for 5 mins. CSFE loaded cells were washed with PBS 10% FCS then resuspended at 1×106 cells/ml in complete medium (RPM1640 2% (v/v) Hepes 1% (v/v) L Glutamine 1% (v/v) penicillin streptomycin) 10% donor's plasma. Cells (2×10⁶ in 2 mL) were added to each well of a 24 well plate pre coated with CH2811hG1 antibody (5 μg/ml), FG2811mG1 (5 μg/ml), CH2811hG2 antibody (5 μg/ml) or containing anti CD3 antibody (OKT 3; 0.005 μg/ml), anti-CD3e Ab (1 ug/ml, eBioscience, 16-0031-85) and anti-CD28 Ab (1 ug/ml eBioscience 16-0281-85) or medium alone. Cells were harvested at day 7, 11 and 14 and stained with relevant antibodies against CD3 (eBioscience, 17-0031), SCA-1 (Miltenyi, 130-102-343), CD62L (Miltenyi, 130-102-543), CD44 (Miltenyi, 130-116-495), anti-CD4-APC-780 (eBioscience 47-0049), anti-CD8-VioGreen (Miltenyi, 130-102-805, Tim3-PE (eBioscience, 130-118-563) or with CH2811hG2-PeCy7 (in house, 1:50 dilution), followed by analysis using MACSQ flow cytometer (MACSQUANT analyser 10).

Luminex [Milliplex Map Kit-Human High Sensitivity T-Cell Magnetic Bead Panel (96 Well Plate Assay)]

Assays in the 9-well format were conducted on filter plates based on the manufacturer's recommendations. In total, 200 μl of wash buffer was added into each well of a 96-well filter plate (Millipore). The plate was sealed and mixed on a plate shaker for 10 mins at room temperature. Wash buffer was removed by inverting the plate and tapping on a paper towel. Then 25 μl of each standard, control and sample (culture supernatant from CSFE proliferation assay) was added into each well, 25 μl of serum matrix was added to each standard and control wells, and 25 μl of assay buffer was added to each sample well. The working bead mix was vortexed immediately before use. Next, 25 μl of the mixed beads was added to each well. The plate was then sealed, wrapped with aluminium foil, and incubated with agitation on a plate shaker (500-800 rpm) for 16-18 hrs at 4° C. After incubation, the plate was rest on a handheld magnet for 60 secs, followed by removing liquid from the plate by inverting the plate and tapping on a paper towel. The plate was washed twice with 200 μl of wash buffer each time. After the second wash, the bottom of the plate was dried by tapping on a paper towel, and 25 μl of detection antibodies was added into each well. The plate was then sealed, wrapped in aluminium foil, and incubated with agitation on a plate shaker for 1 hr at room temperature. Next, 25 μl of streptavidin-phycoerythrin was added to each well containing the 25 μl of detection antibodies. The plate was shaken for an additional 30 mins at room temperature. After incubation, the plate was rest on a handheld magnet for 60 secs, followed by removing liquid from the plate by inverting the plate and tapping on a paper towel. The plate was washed twice with 200 μl of wash buffer each time. Then 150 μl of sheath fluid (Luminex) was added to each well. The beads were resuspended on a plate shaker for 5 mins and read on a Bio-Plex 3D instrument (Bio-Rad, Hercules, Calif.). The instrument was set to collect at least 50 beads per analyte. The raw data were measured as mean fluorescence intensity (MFI).

Naïve T-Cell Isolation

Whole blood was collected from normal donor in syringe contained lithium heparin (1000 units/ml; Sigma H0878). Whole blood was diluted with RPMI 1640 at 1:1 ratio and layered on lymphocyte separation medium (Histopaque-1077; Sigma), followed by centrifugation at 800 g, off brake for 25 mins. After centrifugation, plasma was collected from top layer, PBMCs from the buffy coat layer. PBMCs were washed with RPMI 1640 twice and spun at 317 g for 5 mins. Number of PBMCs was counted and the cells were ready for naive T cell isolation. Every 1×10⁷ PBMCs was resuspended in 40 μl of cold MACS buffer [PBS 1% (v/v) FCS 1% (v/v) EDTA] (PBS: Sigma D8537; FCS: Sigma F9665 and 0.5M pH8 EDTA: Invitrogen). Then 10 μl of Naïve Pan T cell Biotin antibody (Miltenyi) was added to every 1×10⁷ cells and incubated at 4° C., in dark for 5 mins. 30 μl of cold MACS buffer was added to every 1×10⁷ cells followed by 20 μl of Naïve Pan T cell microbead (Miltenyi) to every 1×10⁷ cells and incubated at 4° C., in dark for 10 mins. Cells were added to LS column (Miltenyi) and the flow through, which contained the CD3 purified T cells were collected. Cells retained in the column were non T cells. Naïve T cells were stained with either CH2811hG1 or the combination of CD95/CD122 antibodies for 30 mins. The cells were washed with MACS buffer and proceed to cell sorting. CH2811 hG1⁺ and CD95/CD122⁺ cells were sorted into RNA protect (QIAGEN) and stored at −80° C.

Sample Extraction and Quality Control

8 T-cell samples were provided in RNA protect reagent. The entire sample volume was extracted using the Qiagen RNeasy Plus Mini Kit (Qiagen, Hilden, Germany). Extracted RNA samples were assessed for quantity and integrity using the NanoDrop 8000 spectrophotometer V2.0 (ThermoScientific, USA) and Agilent 2100 Bioanalyser (Agilent Technologies, Waldbronn, Germany) in conjunction with the Eukaryote RNA Pico Bioanalyser chip, respectively. Samples displayed low levels of degradation with RNA integrity numbers (RIN) between 7.4 and 10, and an average yield of 110 ng.

cDNA Synthesis

Full-length cDNA molecules were generated from 1 ng of total RNA per sample using the SMART-Seq® v4 Ultra® Low Input RNA Kit for Sequencing (Clontech, Mountain View, Calif., USA). cDNA quantity was measured using the Qubit® 2.0 Fluorometer (Life Technologies, Carlsbad, Calif., USA), and were checked for quality using the Agilent 2200 Tapestation and high-sensitivity D5000 screentape (Agilent Technologies, Waldbronn, Germany). All samples displayed good quantities of cDNA, with molecule sizes ranging from 400 to 10,000 bp.

Library Generation and RNA-Sequencing Sequencing libraries were prepared using the Illumina Nextera XT Sample Preparation Kit (Illumina

Inc., Cambridge, UK) with an input of 150 μg of cDNA per sample. 11 cycles of final PCR amplification were carried out. Final libraries were quantified and qualified using the Qubit® 2.0 Fluorometer (Life Technologies, Carlsbad, Calif., USA), and the Agilent 2200 Tapestation with a high-sensitivity D1000 screentape (Agilent Technologies, Waldbronn, Germany). Equimolar amounts of each sample library were pooled together for sequencing which was carried out using the Ilumina NextSeq®500 Mid-output kit to generate 75 bp paired-end reads.

Differential Expression Analysis

After quality check using FastQC (http://www.bioinformatics.babraham.ac.uk/projects/fastqc), 75-bp paired-end reads were aligned to the Homosapiens reference genome hg19 using STAR (version 2.6.1d). Mapping was run with default parameters, and reads were counted with GeneCounts. Differential expression analysis (DE) of the 2811- and similarly CD95/CD122-enriched T-cells, was performed using datasets from CD4 and CD8 naïve T-cells from GSE114765 (Pilipow et al. JCI insight 2018) using the edgeR package (version 3.22) followed by Benjamini-Hochberg multiple testing correction to estimate the FDR (FDR <0.05). Common genes between the DE sets (two for CD8 and two for CD4) T-cells were identified using Venny 2.1 and used as input in the StemChecker database to identify a ‘sternness’ signature (Pinto et al. 2015).

Transcriptional Profiling Using RNAseq

Eight T-cell samples (four CH2811⁺, four CD122/CD95⁺) were sorted in RNA protect reagent. The entire sample volume was extracted using the Qiagen RNeasy Plus Mini Kit (Qiagen, Hilden, Germany). Extracted RNA samples were assessed for quantity and integrity using the NanoDrop 8000 spectrophotometer V2.0 (ThermoScientific, USA) and Agilent 2100 Bioanalyser (Agilent Technologies, Waldbronn, Germany) in conjunction with the Eukaryote RNA Pico Bioanalyser chip, respectively. Samples displayed low levels of degradation with RNA integrity numbers (RIN) between 7.4 and 10, and an average yield of 110 ng. Full-length cDNA molecules were generated from 1 ng of total RNA per sample using the SMART-Seq® v4 Ultra® Low Input RNA Kit for Sequencing (Clontech, Mountain View, Calif., USA). cDNA quantity was measured using the Qubit® 2.0 Fluorometer (Life Technologies, Carlsbad, Calif., USA), and were checked for quality using the Agilent 2200 Tapestation and high-sensitivity D5000 screentape (Agilent Technologies, Waldbronn, Germany). All samples displayed good quantities of cDNA, with molecule sizes ranging from 400 to 10,000 bp. Sequencing libraries were prepared using the IIlumina Nextera XT Sample Preparation Kit (IIlumina Inc., Cambridge, UK) with an input of 150 μg of cDNA per sample. 11 cycles of final PCR amplification were carried out. Final libraries were quantified and qualified using the Qubit® 2.0 Fluorometer (Life Technologies, Carlsbad, Calif., USA), and the Agilent 2200 Tapestation with a high-sensitivity D1000 screentape (Agilent Technologies, Waldbronn, Germany). Equimolar amounts of each sample library were pooled together for sequencing which was carried out using the Ilumina NextSeq®500 Mid-output kit to generate 75 bp paired-end reads. After quality check using FastQC (http://www.bioinformatics.babraham.ac.uk/projects/fastqc), 75-bp paired-end reads were aligned to the Homosapiens reference genome (Ensembl assembly GRCh37 (hg19) using STAR (version 2.5.1b). Mapping was run with default parameters, and reads were counted with GeneCounts. Differential expression analysis (DE) of the CH2811- and similarly CD95/CD122-enriched transcription profiles, was performed using datasets from CD4 and CD8 naïve T-cells from GSE83808 (Hosokawa et al. 2017) using the edgeR package (version 3.22) followed by Benjamini-Hochberg multiple testing correction to estimate the FDR (FDR <0.05). Common genes between the DE sets (two for CD8 and two for CD4) T-cells were identified using Venny 2.1 and used as input in the StemChecker database to identify ‘sternness’ signatures and overlap with genesets associated with haematopoetic stem cells (HSC) and embryonic stem cells (ESC) (Pinto et al. 2015). The distribution of the significantly enriched genes was displayed via heatmap analysis (https://software.broadinstitute.org/morpheus/). Concurrently, the distribution of T_{SCM}- and effector T differentiation associated genes, for the CH2811- and CD95/CD122-enriched profiles compared to published naïve and memory CD4 and CD8 T-cells (GSE23321) as well as activated naïve CD8 T-cells (GSE114765) was visualised using http://bioinformatics.sdstate.edu/idep/.

Mouse Study

C57BL/6J mice (Charles River), HHDII/HLA-DP4 (DP*0401) mice (EM:02221, European Mouse Mutant Archive), HHDII mice (Pasteur Institute), aged between 8 to 12 weeks were used. All work was carried out under a Home Office approved project license. Six mice were randomised into two groups (Group A and B) and not blinded to the investigators. Endotoxin free FG2811mG1 mAb were immunised into group A mice (250 μg/mouse) via intraperitoneal route (i.p.) at day 0. Group B mice were used as unimmunised control group. Spleens were harvested for analysis at day 16, followed by pooling splenocytes together within same group and restimulated in the presence or absence of plate bound FG2811mG1 antibody (5 μg/ml). Splenocytes were harvested from culture at day 24, 27 and 30 for analysis using anti-CD3, CD4, CD8, CD44, CD62L, SCA-1 and CH2811hG1 antibodies.

Staining Murine Tissues

Naive HHDII DP4 mice were used. All work was carried out under a Home Office approved project license. Spleens, mesenteric lymph nodes, inguinal lymph nodes, bone marrow and blood samples were from naive mice were harvested for analysis. Tissues were incubated with CH2811hG2-PeCy7 (in house, 1:50 dilution), anti CD3 (eBioscience, 17-0031), SCA-1 (Miltenyi, 130-102-343), CD62L (Miltenyi, 130-102-543), CD44 (Miltenyi, 130-116-495), anti-CD4-APC-780 (eBioscience 47-0049), anti-CD8-VioGreen (Miltenyi, 130-102-805) and Tim3-PE (eBioscience, 130-118-563).

### EXAMPLES

The present invention will now be described further with reference to the following examples and the accompanying drawings.

### Example 1. Generation of FG2811.72

Generation and Characterisation of FG2811 mAb

BALB/c mice were immunised intraperitoneally (i.p.), and boosted intravenously (i.v.) over a period of 3 months with the SSEA-3/-4 expressing cell line (SSEA-3/-4-LMTK). This cell line was produced by transducing wild type LMTK mouse fibroblast cells with α-1-4-galactosyltransferase (A4GALT), β-1-3-N-acetylgalactosaminyltransferase (B3GALNT1) and β-1-3-galactosyltransferase (B3GALT5) genes (Cid et al. 2013). The cell line has endogenous sialyl-transferases, which adds sialic acid at the terminal end of SSEA-3 glycan, producing the SSEA-4 glycan (FIG. 1).

To generate the anti-SSEA-4 specific mAb, splenocytes of immunised mice were fused with myeloma NS0 cells. After repeated rounds of screening and limiting dilution cloning, the anti-SSEA-4 mAb, FG2811mG3 was obtained.

It is known that SSEA-3 and SSEA-4 are globoseries glycolipids. To confirm that FG2811mG3 mAb recognises cell surface glycolipid antigens on SSEA-3/-4-LMTK cells, high performance thin layer chromatography (HPTLC) analysis of SSEA-3/-4-LMTK plasma membrane lipid extracts and immunostaining with FG2811mG3 mAb was performed (FIG. 4A). The MC631 (commercial anti-SSEA-3 mAb) and MC813 (commercial anti-SSEA-4 mAb) mAbs were included as comparison and wild type LMTK cells used as a negative control cell line. Both FG2811mG3 and MC631 mAbs stained glycolipids expressed on SSEA-3/-4-LMTK cells but not wild type LMTK cells. FG2811mG3 mAb showed very specific glycolipid staining whereas MC631 stained three different glycolipid antigens suggesting that the MC631 mAb cross reacted with two other glycolipid antigens expressed on SSEA-3/-4-LMTK cells. MC813 failed to stain glycolipids from both SSEA-3/4-LMTK and LMTK cells, which could be due to its lower affinity towards SSEA-4 antigen. Subsequent cell surface antigen binding showed that MC631 (rat IgM; Gm: 55.93) had the strongest binding to SSEA-3/-4-LMTK cell surface antigen, followed by FG2811mG3 (mouse IgG3; Gm: 20.77) and MC813 (mouse IgG1; 8.77) mAbs (FIG. 4B). Secondary antibody alone (Gm: 0.34) was used as negative control. An ELISA assay was then performed to screen FG2811mG3 mAb against HSA-coupled SSEA-3, SSEA-4, Globo-H, Forssman and Sialyl-Lewis x glycans (FIG. 4C). The results from the ELISA showed that FG2811mG3 mAb was SSEA-4 glycan specific (1.2 OD units) and M1/87 mAb was Forssman glycan specific (1.1 OD units). In contrast, both MC631 and MC813 cross reacted with other glycans. MC631 mAb recognised SSEA-3 (1.0 OD units), SSEA-4 (1.0 OD units) and Globo-H (0.5 OD units) glycans. MC813 mAb bound to SSEA-3 (0.8 OD units), SSEA-4 (1.2 OD units) and Forssman (0.7 OD units). To determine the fine specificity of FG2811mG3, it was screened against 600 natural and synthetic glycans by the Consortium for Functional Glycomics (CFG), FG2811mG3 only bound to SSEA-4 glycan, confirming its specificity towards SSEA-4 (FIG. 4D).

SSEA-3/-4-LMTK plasma membrane lipid antigen binding kinetics of FG2811mG3 mAb was examined using surface plasmon resonance (SPR; Biacore X). Fitting of the binding curves revealed strong apparent functional affinity (K_{d}˜2×10⁻⁹ M) with fast association (˜10⁵ 1/Ms) and slow dissociation (˜10⁻⁴ 1/s) rates for the 2811 mAb (FIG. 5A). This was in line with EC₅₀ values from SSEA-3/-4-LMTK plasma membrane lipid ELISA (EC₅₀=6.8×10⁻¹⁰ M) (FIG. 5B) and cellular functional avidity (K_{d}=5×10⁻⁹M) (FIG. 5C).

### Example 2. FG2811 Antibody Sequence

DNA sequencing revealed that FG2811mG3 mAb belonged to the IGHV2-3*01 heavy chain and IGKV4-63*01 light gene families (FIGS. 2A and B). Assessment of the mutations showed 9 nucleotide differences between the FG2811 heavy chain and the germline sequence, with 5 changes in amino acid residue. Similarly, there were 7 nucleotide differences between the FG2811 kappa chain and the germline sequence, with 5 changes in amino acid residue as a result. The nature and pattern of mutations suggest somatic hypermutation and affinity maturation.

FG2811 heavy and light chain variable regions were cloned into mouse IgG1, human IgG2 and IgG1 expression vectors (FIG. 2C-E). This was transfected into HEK293 cells and antibody purified on protein G. The mIgG3, mIgG1, hIgG1 and hIgG2 mabs bound to SSEA3/4 LMTK cell line (FIG. 3).

### Example 3. 2811 Binding to a Panel of Human Cancer Cell Lines

The overexpression of SSEA-4 have been reported on glioblastoma cancer cell lines, as defined by MC813 mAb. Thus, a panel of brain cancer cell lines were assessed for SSEA-4 expression, using both FG2811mG3 and MC813 mAbs at 5 μg/ml by flow cytometry analysis (FIG. 6A). Mouse IgG3 kappa isotype control and medium alone (no primary) were used as negative controls. The cancer cell line U251 and U87 are adult GBM cells, SF188 and KNS42 are paediatric GBM cells, and UW2283 and DAOY are medulla blastoma cancer cells. FG2811mG3 bound to DAOY (Gm: 50) and UW2283 (Gm: 36) weakly but failed to bind to other cancer cell lines. In contrast, MC813 bound to U251 (Gm: 27) and U87 ((Gm: 38) weakly, DAOY (Gm: 169) and UW2283 (Gm: 152) strongly; failed to bind to KNS42 and SF188. Due to the low specificity of MC813 antibody, this result would suggest that SSEA-4 expression could be found in only DAOY and U251 cell lines and the expression level was low. FG2811mG3 antibody binding to a panel of cancer cell lines composed of ovarian, breast and colorectal cells were further assessed by FACS (FIG. 6B). FG2811mG3 bound strongly to SKOV3 (Gm: 203), moderately to T47D (Gm: 95) and MCF7 (Gm: 76) and weakly to IGROV1 (Gm: 41) and OVCAR-5 (Gm: 87); failed to bind to DU4475 (Gm: 26), HCC1187 (Gm: 22), Colo205 (Gm: 13) and HCT15 (Gm: 22).

### Example 4. Cytotoxicity of 2811 mAb

The ability of FG2811mG3 mAb to induce tumour cell death through ADCC was investigated (FIG. 7A). Human PBMCs were used as the source of effector cells while SKOV3 and T47D cells served as target cells. The number of cells killed by FG2811mG3 mAb was measured after 18 hours incubation at 37° C. The ovarian cancer cells, SKOV3 (EC₅₀: 10⁻¹⁰ M) was susceptible to FG2811mG3 mAb killing in a concentration dependent manner showing a maximum of 66% cell lysis. Despite FG2811mG3 mAb bound to T47D, the mAb failed to induce T47D cell killing via ADCC, suggesting that the killing effect was SSEA-4 expression level dependent. CDC is known to be an important mechanism involves in eliminating tumour cells in vivo. The capacity of the SKOV3 and T47D cells to be killed by CDC induced by FG2811mG3 mAb in the presence or absence of human serum as source of complement was assayed (FIG. 7B). FG2811mG3 mAb showed a maximum of 48% cell lysis of SKOV3 (EC₅₀: 10⁻⁹ M) cells. Again, FG2811mG3 failed to induce T47D cell killing via CDC. To investigate if the FG2811mG3 mAb could induce direct killing on tumour cells, PI uptake assay was carried out using FG2811mG3 mAb at 30 μg/ml with SSEA-3/-4-LMTK and SKOV3 cells (FIG. 7C). Hydrogen peroxide and medium alone were included as positive and negative controls, respectively. FG2811mG3 mAb induced 74.7% of PI uptake on SSEA-3/-4-LMTK and induced weakly; 28.6% on SKOV-3 cells. To confirm that the PI assay truly reflect cell death in growing cells, the cell viability of SSEA-3/-4-LMTK and SKOV3 cells treated with FG2811mG3 mAb at 30 μg/ml were evaluated under light microscope (FIG. 7D). LMTK wild type cell and cells treated with medium alone (RPMI) were used as negative controls. SSEA-3/-4-LMTK cells were observed to aggregate within seconds after FG2811mG3 mAb was added. However, this phenomenon did not develop when SKOV3 and LMTK cells were incubated with FG2811mG3 mAb. FG2811mG3-treated SSEA-3/-4-LMTK and SKOV3 cells showed evidence of growth inhibition after 72 hours of mAb addition. FG2811mG3 mAb showed no effect on LMTK cells. Cells incubated with medium alone did not show growth inhibition and achieved 100% confluent with some cell death over the 72 hours incubation period.

### Example 5. 2811 Staining of Erythrocytes

Both SSEA-3 and SSEA-4 were reported to be expressed on the erythrocytes of the majority of people. Therefore, binding of FG2811mG3 mAb at a range of concentrations (10 μg/ml) to erythrocytes from 5 donors was assessed by flow cytometry (FIG. 8A). Anti-CD55 mAb (791T/36; 10 μg/ml; Gm: 202) was included as positive control whereas IgG isotype control and PBS were used as negative control. FG2811mG3 (Gm: 10) did not bind to erythrocytes from all 5 donors. Erythrocyte agglutination assay further confirmed that FG2811mG3 mAb (0.625 to 10 μg/ml) did not agglutinate erythrocytes from 5 donors. In contrast, 791T/36 mAb and anti-blood serum antibodies agglutinated erythrocytes from all donors. PBS was used as negative control (FIG. 8B).

### Example 6. 2811 Binding to Stem Memory T-Cells (T_{SCM})

The discovery of T_{SCM} cells and the fact that SSEA-4 is a stem cell marker leads us to hypothesise that 2811 mAb may recognise T_{SCM} cells. Whole blood samples were collected from seven healthy donors (BD3, BD13, BD18, BD27, BD38, BD96, BD31) and stained with FG2811mG1 mAb (FIG. 9A). The MC813 mAb was included as comparison; the mouse IgG1 isotype control antibody and secondary antibody alone (no primary) we used as negative controls; the 198 antibody (anti-CEACAM6) and OKT3 antibody (anti-CD3) were used as positive control antibodies for granulocytes and PBMCs, respectively. FG2811mG1 mAb stained a small population of peripheral blood mononuclear cells (PBMCs), ranging from 0.8 to 2.3% among the seven healthy donors. The MC813 mAb which recognises SSEA3, SSEA4 and Forssman antigens did not stain any blood cells across seven donors. The 198 mAb stained granulocytes and the OKT3 mAb stained CD3⁺ T-cells. The secondary antibody and medium alone showed no cell staining. Next, to investigate whether these 2811⁺PBMCs were T_{SCM} cells, PBMCs were collected from two healthy donors, co-stained with FG2811, CD3, CD122, CD45RA, CD45RO, CD62L and CD95 antibodies and analysed by multiparameter flow cytometry (FIG. 9B, Table 2). The CD3⁺total T-cells were first identified, followed by the identification of 2811⁺ population. The frequency of 2811⁺ cells ranged from 0.32 to 0.41% across the two donors. Subsequently, the expression of CD45RA and CD45RO markers were analysed from the CD3⁺2811⁺ population. The CD3⁺2811⁺ T cells were composed of CD45RA⁺ (37.5-38.6%), CD45RO⁺(38-47.8%) and CD45RA⁺RO⁺ (12.7-23.1%) cell subsets. Finally, the expression of CD62L, CD95 and CCR-7 were assessed from the CD45RA⁺, CD45RO⁺ and CD45RA⁺RO⁺ populations. The majority of CD45RA⁺(88.7-90%), CD45RA⁺RO+(79.5-89.6%) and CD45RO⁺(64.5-67.1%) were CD62L⁺; 27.6-59.7% of CD45RA⁺, 29.5-85.7% of CD45RA⁺RO⁺ and 81.2-86.1% of CD45RO⁺ cells were CD95⁺ and 56.1-78.9% of CD45RA⁺, 51.4-78.1% of CD45RA⁺RO⁺ and 53.7-61.2% of CD45RO⁺ cells were CCR-7⁺. These results suggested that 2811/CD45RA⁺ cells were T_{SCM} cells whereas 2811/CD45RA⁺RO⁺ cells could be activated T_{SCM} and 2811/CD45RO⁺ cells could be activated T_{SCM} or T_{CM} cells.

TABLE 2
PBMCs phenotyping. PBMCs were isolated from two healthy donors
(BD13 and BD38) and stained with a panel of antibodies (CD3,
FG2811, CD45RA, CD45RO, CD62L, CD95 and CCR-7). Phenotype
of the PBMCs were determined using flow cytometry, and results
were presented as the percentage of positive cells.

Donor

| Percentage (+) cells | BD13 | BD38 |
|---|---|---|
| CD3+2811+ | 0.41 | 0.32 |
| CD3+2811+CD45RA+ | 38.6 | 37.5 |
| CD3+2811+CD45RA+RO+ | 12.7 | 23.1 |
| CD3+2811+CD45RO+ | 47.8 | 38 |
| CD3+2811+CD45RA+CD62L+ | 90 | 88.7 |
| CD3+2811+CD45RA+RO+CD62L+ | 79.5 | 89.6 |
| CD3+2811+CD45RO+CD62L+ | 64.5 | 67.1 |
| CD3+2811+CD45RA+CD95+ | 27.6 | 59.7 |
| CD3+2811+CD45RA+RO+CD95+ | 29.5 | 85.7 |
| CD3+2811+CD45RO+CD95+ | 86.1 | 81.2 |
| CD3+2811+CD45RA+CCR-7+ | 56.1 | 78.9 |
| CD3+2811+CD45RA+RO+CCR-7+ | 51.4 | 78.1 |
| CD3+2811+CD45RO+CCR-7+ | 61.2 | 53.7 |

In the hierarchical model of human T-cell differentiation, after antigen priming, naïve T-cells (T_{N}) progressively differentiate into stem memory T-cells (T_{SCM}), central memory T-cells (T_{CM}), effector memory T-cells (T_{EM}) and ultimately into terminally differentiated effector T-cells (T_{TE}/TEMRA). These T-cell subsets are distinguished by the combinatorial expression of different markers (Table 3) (Gattinoni et al. 2017).

TABLE 3
Hierarchical model of human T-cell differentiation.

| | Progressive T-cell differentiation model | | | | |
|---|---|---|---|---|---|
| | T_{N} | T_{SCM} | T_{CM} | T_{EM} | T_{TE}/TEMRA |
| CD45RA | + | + | − | − | + |
| CD45RO | − | − | + | + | − |
| CCR-7 | + | + | + | − | − |
| CD62L | + | + | + | − | − |
| CD28 | + | + | + | +/− | − |
| CD27 | + | + | + | +/− | − |
| IL-7Rα | + | + | + | +/− | − |
| CXCR3 | − | + | + | − | − |
| CD95 | − | + | + | + | + |
| CD11a | − | + | + | + | + |
| IL-2Rβ | − | + | + | + | + |
| CD58 | − | + | + | + | + |
| CD57 | − | − | − | +/− | + |

### Example 7. RNA Sequencing of 2811 Positive T-Cells

By transcriptome analysis, we investigated the degree of relatedness between putative T_{SCM} cells (Gattinoni et al. 2017) and 2811⁺ T-cells. The CD95 and CD122 (IL-2Rβ) markers discriminate T_{N} cells to T_{SCM} cells; the CD45RO marker distinguishes other memory T-cell subtypes from T_{SCM} cells. Thus, naïve T-cells were isolated from four healthy donors using Pan naïve human T-cell isolation kit (Miltenyi), which contained a cocktail of biotinylated antibody for the depletion of memory T-cells and non-T-cells. The purified naïve T-cells (CD45RA⁺) were subsequently stained with CH2811hG1 or the combination of CD95/CD122 to isolate 2811⁺ and putative T_{SCM} cells, respectively. RNA sequencing on CH2811hG1- and CD95/CD122-enriched T-cells and differential gene expression (DE) analysis using a data set from CD8 native T-cells showed that of the 5,036 genes that were significantly up or down regulated in the SSEA-4 positive (CH2811) cells, 2227 (44%) were common with the up or down regulated DE genes in CD95/CD122 positive T-cells suggesting there was a substantial overlap genes between these two populations (FIG. 10A). Of the common genes, 257 significantly overlapped with embryonic stem cells genes sets, 103 with haematopoietic stem cells and 78 with embryonal carcinomas (FIG. 10A), implying that SSEA-4 is indeed associated with a subset of T-cells with stem-like behaviour. The distribution profile of the former two overlapping gene sets in our dataset is shown using heatmap analysis. Additionally, the distribution of T_{SCM}- and effector differentiation gene subsets among our CH2811hG1- and CD95/CD122-enriched T-cell profiles and the comparison with CD8/CD4 naïve and memory T-cells as well as activated CD8 native T-cells (‘donor’) is also shown (FIG. 10B). Hierarchical clustering shows a clear separation of the CH2811 hG1 and CD95/CD122 samples, suggesting they are more similar to each other compared to bona fide naïve/memory or activated naïve T-cells and may represent a distinct T-cell subset with stem-like behaviour (FIG. 10C).

### Example 8. T-Cell Proliferation and Expansion

According to the paradigm of co-stimulation, T_{N} cells require the engagement of both T-cell receptor (TCR) signal 1 and costimulatory signal 2 for complete activation leading to proliferation and differentiation. However, a subclass of CD28 specific antibodies known as CD28 superagonists, which unlike conventional CD28 antibodies, are capable of fully activating T-cells without additional stimulation of TCR. We investigated whether CH2811hG1 mAb is capable of inducing CD4 and CD8 T-cell proliferations. Initially, PBMCs were isolated from two healthy donors (BD3 and BD18) and CSFE labelled followed by antibody stimulation using plate bound CH2811hG1 mAb at 5 μg/ml, which showed proliferation of both CD4 (13-20%) and CD8 (2-31%) T-cells at day 11 (FIG. 11A-B). PBMCs stimulated with PHA and medium alone were positive and negative controls.

To obviate that this was due to Fc activation of antigen presenting cells, purified T-cells (96% purity; FIG. 12A) were isolated from 4 healthy donors, CSFE labelled followed by stimulation with plate bound CH2811hG1 mAb at 5 μg/ml. At day 14, 8-18% of CD4⁺ T-cells and 3-7% for CD8⁺ T-cells proliferated, suggesting that the proliferation was not mediated by Fc interaction (FIG. 12B-C). Cells stimulated with anti-CD3 mAb (0.005 μg/ml) and medium alone were used as positive and negative controls, respectively. The percentage of cells undergoing cell division varied as most of the SSEA4 positive cells had undergone at least 4 cell division (96%) in the 14-day period whereas only 55% of the CD3 stimulated cells had undergone 4 cells divisions (FIG. 12D)

### Example 10. Assessment of TCR Repertoire Clonotype

The clonality of the CH2811IgG1 stimulated T cells was assessed from 2 donors (BD3 and BD26), The TCR repertoire was determined, a fully automated multiplex PCR was performed to generate TCRα (TRA) and TCRβ (TRB) chain libraries for next generation sequencing (NGS) analysis of unique CDR3s (uCDR3). A tree plot analysis (FIG. 13) revealed the presence of some relatively dominant clonotypes in the CSFE low populations of both donors. The diversity of the non-proliferation population was 18.9 and 12.8 respectively for TRA and TRB chains respectively. As expected, the diversity of the 2811 stimulated population was 3 and 3.3 for TRA and TRB chains respectively. The diversity was less suggesting that these cells represent antigen experienced cells.

### Example 11. Dynamic of Individual Cytokine/Chemokine Responses

T_{SCM} cells have been shown to have high proliferative capacity and are both self-renewing and multi-potent, in which they can further differentiate into other T-cell subsets. We hypothesised that FG2811⁺ T_{SCM} cells could proliferate and self-renew in vitro in the absence of any supplemental cytokines. We first aimed to identify the cytokines released by FG2811⁺ T_{SCM} cells following CH2811hG1 antibody stimulation, then design a method that can be used to expand and maintain the stemness of putative FG2811⁺ T_{SCM} cells. T-cells were purified from four healthy donors and stimulated with plate bound CH2811hG1 mAb, supernatant was collected at day 7, 11 and 14 and assessed for cytokines or chemokines release. Unstimulated cells (media only) were used as negative control. Secretion of nine cytokines/chemokines (IFNγ, IL-10, IL-17A, IL-2, IL-21, IL-5, IL-7, IL-8 and TNFα) was assayed using a multiplexed cytokine assay (Luminex technology) (FIG. 14). Following CH2811hG1 mAb stimulation, the chemokine IL-8 was strongly upregulated whereas more modest levels of TNFα, IL-10 and IL-5 were detectable from day 7 to 14.

The unstimulated and the anti-CD3 stimulated T-cells did not survive in culture beyond day 14, only CH2811hG1 stimulated T-cells survived beyond 14 days (FIG. 15A) as shown by trypan blue exclusion. At day 35, the viable CH2811hG1 stimulated T-cells were collected and characterised by staining the cells with a panel of antibodies (FG2811, CD3, CD122, CD45RA, CD45RO, CD62L and CD95) and analysed using multiparameter flow cytometry (FIG. 15B). Of the 32.47% viable cells, 3% were FG2811⁺ and the remaining 97% were FG2811⁻. The FG2811⁺ cells [FIG. 15B(i)] were 99% CD3⁺ and CD122⁺, of which 60% was CD45RA/RO double positives (CD45RA/RO⁺) and 30% was CD45RA⁺. Both CD45RA/RO⁺ and CD45RA⁺ cells were CD62L⁺ and CD95⁺, suggesting that they are TSCM cells. The CD45RA/RO⁺ population could be the activated TSCM cells whereas the CD45RA⁺ could be the more naïve like TSCM cells. The FG2811⁻ population [FIG. 15B(ii)] was 99% CD3⁺ but only 34% was CD122⁺. The FG2811⁻CD3⁺ population was 62% CD45RO⁺ and 17% CD45RA⁺. CD62L was expressed on 49% FG2811⁻CD3⁺CD45RO⁺ cells and CD95 was expressed on 79% of the FG2811⁻CD3⁺CD45RO⁺ cells. The CD45RO/CD62L/CD95 triple positives cells (CD45RO/CD62L/CD95⁺) could be the activated T_{SCM} or T_{CM} cells whereas the CD45RO/CD95⁺ cells could be TEM or TEMRA. The CD45RA⁺ population contained more CD62L⁺ cells (˜76%) but fewer CD95⁺ cells (˜28%). The CD45RA/CD62L/CD95⁺ cells could be T_{SCM} cells. This result suggests that CH2811hG1 stimulation maintains T-cells with ‘stem-like’ and memory characteristics in culture for a long period, and these may differentiate into other T-cell types. The proliferative potential of these viable cells was assessed by re-stimulating them with anti-CD3/CD28 antibodies at day 33 or with CH2811hG1 mAb at day 33 and day 64. Under light microscope, at day 39, cells re-stimulated with anti-CD3/CD28 antibodies formed T-cell blasts [FIG. 15C (i)], which majority of CD3/CD28 re-stimulated T-cells were dead, with only a few viable cells remaining. In contrast, cells re-stimulated twice with CH2811 antibody remained viable at day 70 and showed an obvious expansion in numbers [FIG. 15C (ii)]. The supernatant from these two cultures were collected at day 39, 54 and 70 and screened for cytokines and chemokines (FIG. 15D). In the CH2811hG1 re-stimulated culture, while other cytokines/chemokine levels decreased gradually from day 14 to undetected level by day 70, IL-7 and IL-21 levels increased gradually [FIG. 15D (i)]. This result suggests that IL-7 and IL-21 could play an important role in self-sustaining FG2811⁺ T_{SCM} cells in culture, IL-7 is known to provide key instructive signals for T_{SCM} formation (Cieri et al. 2013) whereas IL-21 plays an crucial role in inhibiting effector T-cell differentiation (Lugli, Dominguez, et al. 2013). In the anti-CD3/28 antibody re-stimulated culture, all cytokines and chemokines increased, suggesting that there was an activation of a variation of different T-cell subsets [FIG. 15D (ii)]. For instance, Th1 cells are characterised by the secretion of IL-2, IFNγ and TNFα, Th2 secretes IL-5, Th17 secretes IL-17A and IL-21, regulatory T-cells (Tregs) secretes IL-10 (Raphael et al. 2015).

### Example 12. Identification of FG2811⁺ T_{SCM} Cells in Mice

Next, we investigated the expression of SSEA-4 on mouse splenocytes, mesenteric and inguinal lymph node cells using the CH2811hG1 antibody (FIG. 16). These results showed that CH2811hG1 antibody stained 0.5% of splenocytes, 0.37% of mesenteric lymph node cells and 0.52% of inguinal lymph node cells.

### Example 13. FG2811 (Mouse IgG1) Induces Phenotypic T_{SCM} Cells in C57B/6J Mice

To determine the T-cell agonistic effect of FG2811mG1 in vivo, a group of 3 mice (Group A) were immunised i.p. with FG2811 at 250 μg at day 0 (Group A). Three unimmunised mice were included as control group (Group B). At day 16, mice from both groups were euthanised and spleens were harvested. The total cell number of splenocytes from Group A was higher compared to Group B mice, ranged from 7×10⁷ to 1×10⁸ cells and 3.9×10⁷ to 6.2×10⁷ cells, respectively (FIG. 17A). Splenocytes from individual mouse within each group were stained with CH2811hG1, anti-CD4, CD8, CD19, SCA-1, CD44, CD62L, CD11 b and F4/80 antibodies and analysed by multiparameter flow cytometry (FIG. 17B). CH2811hG1 mAb was used to identify SSEA-4⁺ splenocytes, anti-CD4 and CD8 for T-cells, CD44 and CD62L for T and B cell subsets, SCA-1 (marker used to identify hematopoietic stem cells and mouse T_{SCM} cells along with other markers) for stem cell-like cells and CD11b and F4/80 for macrophages. The 2811⁺(0.97-1.2%), CD62L⁺(5.51-10.83%) and CD62⁺CD44⁺(8.74-15.03%) cell frequencies were lower in group A mice compared to group B 1.62-1.74%, 17.39-19.2% and 18.4-27.34%, respectively. The differences in percentage of CD4⁺, CD8⁺, CD19⁺, CD11b+, F4/80⁺ and CD11b+F4/80⁺ cells between both groups were minimal, except that mouse A3 contained lower CD8⁺ T-cell population (Table 4). This result could suggest that FG2811mG1 antibody immunisation induced 2811⁺ cell proliferation and differentiation, which lead to the reduction of naïve like cells (2811⁺, SCA-1⁺ and CD62L⁺) in vivo.

TABLE 4
Summary of the frequencies of different immune cell
subsets in Group A and B splenocytes at day 16.

| Marker | 2811 immunised (Group A) | Control (Group B) |
|---|---|---|
| 2811 | 0.97-1.2% | 1.62-1.74% |
| SCA-1 | 37.23-55.06% | 42.22-61.73% |
| CD62L | 5.51-10.83% | 17.39-19.2% |
| CD62L, CD44 | 8.74-15.03% | 18.4-27.34% |

Splenocytes from each group were pooled together and then cultured in the presence (A+2811 and B+2811) or absence (A-2811 and B-2811) of 5 μg/ml of plate bound FG2811mG1 mAb. Subsequently, at day 24, 27 and 30, these cells were harvested and stained with FG2811, CD3, CD4, CD8, CD44, CD62L, SCA-1, CD11b, F4/80 and CD19 antibodies and analysed by multiparameter flow cytometry (FIG. 17C-D). At day 24, Group A splenocytes re-stimulated with (A+2811) or without (A-2811) FG2811mG1 mAb, formed small- and large-sized cell populations as indicated by forward and side scatter (FSC/SSC) profiles. In contrast, Group B splenocytes re-stimulated with (B+2811) or without (B-2811) FG2811mG1 mAb did not generate the large-sized population (FIG. 17C). The large-sized population continued to persist in A+2811 and A-2811 splenocyte cultures till day 30 (FIG. 17D). The large-sized cell population mainly consists of CD3^{moderate-high} (CD3^{mo-hi}) CD4^{high} (CD4^{hi}) and CD8^{high} (CD8^{hi}) T-cells, whereas the small-sized cell population consists of CD3^{low-moderate} (CD3^{lo-mo}) CD4^{low})(CD4^{lo} and CD8^{low} (CD8^{lo}) T-cells.

In the hierarchical model of mouse T-cell differentiation, after antigen priming, naïve T-cells (T_{N}) progressively differentiate into stem memory T-cells (T_{SCM}), central memory T-cells (T_{CM}), effector memory T-cells (T_{EM}). These T-cell subsets are distinguished by the combinatorial expression of different markers (Table 5).

TABLE 5
Phenotypic markers of murine T-cell populations

CD3+

| | T_{N} | T_{SCM} | T_{CM} | T_{EM} |
|---|---|---|---|---|
| CD44 | − | −/+ | + | + |
| CD62L | + | + | + | − |

Phenotypic analysis revealed that the CD3^{mo-hi} population in A+/−2811 cultures mainly consists of T-cells with CD44⁻CD62L⁺(T_{N} and/or T_{SCM}; 28.8-32.49%) and CD44⁺CD62L⁺(T_{CM}; 37.92-41.08%) phenotypes, followed by CD44⁺CD62L⁻ (T_{EF}/T_{EM}; —27.8%) phenotype and a small fraction of cells with CD44⁻CD62L⁻ phenotype (1.72-2.26%). In contrast, the CD3^{lo-mo} population in all cultures mainly consists of CD44⁺CD62L⁻ (T_{EF}/T_{EM}; 66.09-70.83%) phenotype followed by CD44⁻CD62L⁻ phenotype (26.77-32.17%). The percentages of T_{N} and/or T_{SCM} cells and T_{CM} cells were between 0.08-0.24% and 1.5-2.29%, respectively. In addition to T-cells, the large-sized cell population also contained CD19^{hi}, CD62L⁺ and CD62L⁺SCA-1⁺ cells, which were all absence from the small-sized cell population. Only CD19^{lo} cells were detected in the small-sized cell population. Interestingly, the percentage of CD11b⁺F4/80⁺ macrophage population was significantly reduced in the A+/−2811 groups. FG2811mG1 antibody stimulation in vitro of splenocytes from unimmunised mice splenocytes B+2811 culture did not form these large-sized population even by day 30, indicating that the generation of this cell population was an in vivo FG2811 antibody immunisation effect.

### Example 14. Identification of T_{SCM} Cells in HHDII and HHDII Transgenic Mice

To determine the frequency of T_{SCM} cells in HHDII (FIGS. 18A and B) and HHDII/DP4 mice (FIG. 18 C-E), splenocytes were harvested from naive mice and stained with CH2811hG2-PeCy7, anti CD3, CD4, CD8, CD44, CD62L and SCA-1 then analysed by multiparameter flow cytometry. CH2811hG2 mAb was used to identify SSEA-4⁺ splenocytes, anti-CD4 and CD8 for T-cells, CD44 and CD62L for T cell subsets, SCA-1 (marker used to identify hematopoietic stem cells and mouse T_{SCM} cells along with other markers) for stem cell-like cells. In HHDII mice 10.88% of cells were 2811+CD3+ cells this translated into 1.85×10⁵ cells per ml, in addition 24.61% of the CD3+ population were T_{SCM} cells (FIG. 18B). The 2811⁺ population (10.88%) in HHDII mice was higher than the frequency previously observed in C57/B6 mice (2.42-3.60%). Further phenotypic analysis (FIG. 18B) of the 2811+ population in HHDII mice showed that 33.38% CD44+CD62L− and 47.98% were CD44+CD62L+. The percentage of 2811+ cells that expressed the stem cell marker, SCA-1, was also determined, this marker when used in combination with other markers (CD44-CD62L+) can define T_{SCM} cells in these mice. The majority of 2811+SCA-1+ cells also express CD44 suggesting that they are antigen experienced.

In HHDII/DP4 mice 12.01% of cells were 2811+CD3+ cells this translated into cells per 0.91×10⁵ ml, in addition 6.98% of the CD3+ population was also 2811+(FIGS. 18C and D). The 2811+ population (12.01%) in HHDII/DP4 mice similar to the frequency in HHDII mice and was also higher than the frequency previously observed in C57/B6 mice (2.42-3.60%).Further phenotypic analysis (FIG. 18D) of the 2811+ population in HHDII/DP4 mice showed that 12.09% were CD44+CD62L− and 77.15% were CD44+CD62L+. The percentage of 2811+ cells that expressed the stem cell marker, SCA-1, was also determined. The majority of 2811+SCA-1+ cells also express CD44 (75.51% CD44+CD62L+, 30.03% CD44+CD62L−).

A more detailed phenotypic analysis was performed on the T cell populations from HHDII/DP4 mice, this analysis looked at the expression of 2811 in the CD4 and CD8 T cell subsets but also looked at the expression of the exhaustion marker, Tim3. The percentage of CD4+ T cells in the HHDII/DP4 mice was 14.30%, however, the percentage of CD8+ T cells was very low with only 0.50% CD8+ cells (FIG. 18E), of the CD4 T cells 9.47% were 2811+, and of the CD8 T cells 10.14% were 2811+. The expression of the exhaustion marker, Tim3, was low on CD4+2811+(0.42%) cells and CD8+ 2811+(0.34%) cells in line with their stem cell properties.

### Example 15. Plate Bound Human (IgG1) and Mouse (IgG1) 2811 Induced Ex Vivo Proliferation of Mouse Splenocytes

We investigated whether plate bound CH2811hG1 and FG2811mG1 mAb are capable of inducing CD4 and CD8 T cell proliferation. Splenocytes were harvested from HHDII naive mice, pan T cells enriched (CD3+) and labelled with CFSE, followed by antibody stimulation using plate bound CH2811hG1 mAb or FG2811mG1, anti CD3 was used as a positive control and media as a negative control (FIG. 19A). The proliferative responses of the CD3, CD4 and CD8 T cell populations was determined on days 7, 12 and 14 (FIG. 19 B-D). The results showed that CD3, CD4 and CD8 T cells proliferated in response to stimulation with plate bound CH2811hG1 and FG2811mG1 mAb, this was equal to or slight above the media control. On day 7, for CD3 T cells 2.72% proliferated in response to FG2811mG1 and 2.24% proliferated in response to CH2811hG1, for CD8 T cells 2.47% proliferated in response to FG2811mG1 and 1.46% proliferated in response to CH2811hG1, for CD4 T cells 1.32% proliferated in response to FG2811mG1 and 0.96% proliferated in response to CH2811hG1. On day 12 the proliferative response to plate bound CH2811hG1 and FG2811mG1 had increased, for CD3 T cells 6.46% proliferated in response to FG2811mG1 and 6.27% proliferated in response to CH2811hG1, for CD8 T cells 6.21% proliferated in response to FG2811mG1 and 3.33% proliferated in response to CH2811hG1, for CD4 T cells 5.79% proliferated in response to FG2811mG1 and 2.82% proliferated in response to CH2811hG1. On day 14 the proliferative response to plate bound CH2811hG1 and FG2811mG1 had increased further, for CD3 T cells 10.07% proliferated in response to FG2811mG1 and 8.7% proliferated in response to CH2811hG1, for CD8 T cells 7.87% proliferated in response to FG2811mG1 and 6.61% proliferated in response to CH2811hG1, for CD4 T cells 7.29% proliferated in response to FG2811mG1 and 5.15% proliferated in response to CH2811hG1. These results show that murine splenocytes proliferate ex vivo in response to plate bound CH2811hG1 and FG2811mG1 mAb.

### Example 16. Anti CD3 and CD28 Induces the Ex Vivo Expansion of 2811+ Cells from HHDII Mice

We investigated whether anti CD3 and anti CD28 could induce the proliferation of 2811+ cells isolated from HHDII mice. Splenocytes were harvested from HHDII naive mice, pan T cells enriched (CD3+) and labelled with CFSE, followed by stimulation with anti CD3 and anti CD28 (1 μg/mL). The proliferative responses of the 2811+ population was determined on days 11, 15 and day 20 using CH2811hG2-PeCy7 mAb (FIG. 20A). The percentage of 2811+CD3+ T cells increased following stimulation with anti CD3 and anti CD28, by day 11 61.2% of T cells were 2811+, by day 15 this had increased further to 69.84%, but by day 20 the percentage 2811+ T cells had reduced to 57.58%. The percentage decrease in 2811+ cells observed on day 20 also correlated with a decrease in cell viability with a reduction in the total number of T cells and 2811+ T cells (FIG. 20Aiii and iv). The percentage of 2811+ cells in the media only control was 10% direct ex vivo, this increased to 20-30% at day 11 and 15 but also decreased at day 20.

Phenotypic analysis was performed on the 2811+ cells that had expanded following stimulation with anti CD3 and anti CD28. Staining was performed on day 11 (FIG. 20B) using CH2811hG2-PeCy7, anti CD3, CD44 and CD62L. The T cell subsets identified were effector memory T cells (T_{EM}) as defined by CD44+CD62L−, central memory T cells (T_{CM}) as defined by CD44+CD62L+, effector T cells (T_{EFF}) as defined by CD44−CD62L− and naive T cells (T_{N}) as defined by CD44−CD62L+. The phenotyping results at day 11 (FIG. 20C) show that stimulation with anti CD3 and CD28 increased the total number of 2811+T_{EM} (mean 67.35×10³), T_{CM} (mean 61.15×10³), T_{EFF} (mean 141×10³) and T_{N} (mean 16.45×10³). Stimulation with anti CD3 and anti CD28 pushed the phenotype of the 2811+ cells to a more effector T cell phenotype (FIG. 20D). The percentage of 2811+, T_{EFF} cells was 47.7% (mean value), whereas the percentage of T_{CM} and T_{EM} cells had reduced to a percentage below the unstimulated cells (media alone).

These results show that anti CD3 and anti CD28 induces the ex vivo expansion of 2811+ cells from HHDII mice. Stimulation with anti CD3 and anti CD28 led to an increase in the number and percentage of 2811+ cells 11-15 days post stimulation. The total number of 2811+ T cells within each subset increased (T_{CM}, T_{N}, T_{EM}, T_{EFF}), however, the stimulation did push T cells into a more effector T cell phenotype.

### Example 17. Human (IgG2) and Mouse (IgG1) 2811 Induced Ex Vivo Proliferation of Splenocytes from HHDII/DP4 Mice

We investigated whether plate bound CH2811hG2 and FG2811mG1 mAb are capable of inducing CD4 and CD8 T cell proliferation. Splenocytes were harvested from HHDII naive mice, pan T cells enriched (CD3+) and labelled with CFSE, followed by antibody stimulation using plate bound CH2811hG2, FG2811mG1, anti CD3/CD28 (+/− AKTi) was used as a positive control, media as a negative control. The proliferative responses of the CD3 T cell population was determined on days 11, 15 and 20 (FIG. 21A). The results showed that 2811+CD3 T cells proliferated in response to stimulation with plate bound CH2811hG2 and FG2811mG1 mAb. On day 11, 8.73% 2811+CD3+ T cells proliferated in response to FG2811mG1 and 50.47% in response to CH2811hG2, on day 15 20.48% 2811+CD3+ T cells proliferated in response to FG2811mG1 and 40.55% in response to CH2811hG2, by day 20 the percentage reduced slightly with 21.41% 2811+CD3+ T cells proliferated in response to FG2811mG1 and 35.13% proliferated in response to CH2811hG2. The same increase in 2811+ cells was seen when also looking at the percentage of 2811+ cells, total number of 2811+CD3+ and 2811+ cells. Stimulation with anti CD3/CD28 with or with AKTi also induced proliferation of 2811+ cells, with 80% 2811+CD3+ T cells at each time point following CD3/CD28 stimulation, this percentage dropped to 60% with the addition of AKTi, which was slightly toxic to the cells. These results show that CH2811hG2 induced the proliferation of 2811+ cells at all time points following stimulation, the same was also seen with FG2811mG1 but to a lesser extent.

Phenotypic analysis was then performed on the 2811+ cells that had expanded following stimulation with anti CD3/CD28 (+/−AKTi), CH2811hG2 and FG2811mG1 mAbs. Staining was performed on day 11 (FIG. 20B) using CH2811hG2-PeCy7, anti CD3, CD44 and CD62L. The T cell subsets identified were effector memory T cells (T_{EM}) as defined by CD44+CD62L−, central memory T cells (T_{CM}) as defined by CD44+CD62L+, effector T cells (T_{EFF}) as defined by CD44−CD62L− and naive T cells (T_{N}) as defined by CD44-CD62L+. The phenotyping results at day 11 (FIG. 21B) showed that stimulation with CH2811hG2 or FG2811mG1 mAb increased the total number of 2811+T_{EM} to 62.8×10³ cells following CH2811hG2 stimulation and 5.24×10³ following FG2811mG1 stimulation. The total number of 2811+T_{CM} increased to 6.95×10³ cells following CH2811hG2 stimulation and 1.8×10³ following FG2811mG1 stimulation. The total number of 2811+T_{EFF} increased to 29.05×10³ cells following CH2811hG2 stimulation and 7.02×10³ following FG2811mG1 stimulation, there was only slight increases in the total number of 2811+T_{N} cells which increased to 0.61×10³ cells (media only 0.07×10³) following CH2811hG2 stimulation and there was no increase following FG2811mG1 stimulation. We also looked at the percentage of 2811+ T cells following stimulation with anti CD3/CD28, FG2811mG1 and CH2811hG2 (FIG. 21C), the percentage of 2811+T_{EFF} cells was 34.82% following stimulation with FG2811mG1, 57.59% following stimulation with CH2811hG2 and 47.36% following stimulation with anti CD3/CD28. These results show that in T cells from HHDII/DP4 mice there is less skewing of the T cells into an effector phenotype when compared the results obtained from the HHDII mice (FIG. 20D). The percentage of the T_{CM} and T_{EM} subsets was also higher in the HHDII/DP4 mice when compared with the HHDII mice.

These results show that splenocytes from HHDII/DP4 mice proliferate ex vivo in response to plate bound CH2811hG2 and FG2811mG1 mAb, this leads to an increase in the total number of 2811+CD3+ T cells in addition to increases in the number of 2811+ effector memory, central memory, effector and naive T cells. The magnitude of the proliferative ex vivo response to CH2811hG2 was larger when compared to the response to FG2811mG1 thus leading to a higher number of 2811+ cells.

### Example 18. Anti CD3 and CD28 Induces the Ex Vivo Expansion of 2811+ Cells from Healthy Donors

TSCM cells have been shown to have high proliferative capacity and are both self-renewing and multi-potent, in which they can further differentiate into other T-cell subsets. We investigated if anti CD3/CD28 stimulation or the addition of different cytokines could induce the ex vivo proliferation of Tscm cells isolated from healthy donors. PBMCs were isolated from 4 healthy donors (buffy coats), a pan T cell enrichment was performed, T cells were cultured in the presence of anti CD3/CD28, IL-7, IL-15 or IL-21. Phenotypic analysis was performed on days 15 and 20 using anti CD3, CD45RA, CD45RO, CD62L, CD95, CD122 and CCR7, the expression of different makers used to identify T cell populations are listed in table 6.

TABLE 6
Phenotypic markers of human T-cell populations

| | CD3+ | | | | | |
|---|---|---|---|---|---|---|
| | CD45RA | CD45RO | CD62L | CD95 | CD122 | CCR7 |
| T_{N} | + | | + | | | + |
| T_{SCM} | + | (+) | + | + | + | + |
| T_{CM} | | + | + | + | + | + |
| T_{EM} | | + | | + | | − |
| T_{EMRA} | + | | | + | + | − |

Phenotypic analysis was performed on the CD3+ T cells that had expanded following stimulation with anti CD3/CD28 or with the addition of IL-7, IL-15 and IL-21 added in a range of combinations. Staining was performed on day 15 and day 20 (FIG. 22A). The phenotyping results at day 15 (FIG. 22B) showed that stimulation with CD3/CD28 alone or in combination with IL-7, IL-15 and IL-21 increased the percentage of 2811+CD3+ cells, this also correlates with an increase in the total number of 2811+ and CD3+ cells. On day 15 the percentage of 2811+CD3+ T cells following stimulation with anti CD3/CD28 was 19.64% and 23.94%, this was higher than T cells cultured in the presence of cytokines only (no CD3/CD28 stimulation). The addition of IL-7/IL-21 or IL-7/IL-15/IL-21 in combination with anti CD3/CD28 stimulation did slightly improve the percentage of 2811+CD3+ cells. The percentage of 2811+CD3+ T cells increased to 23.8% and 27.4% when cells were cultured in the presence of CD3/CD28, IL-7/IL-21, with the addition of IL-15 the percentage increased to 31.53%. The increase in the percentage of 2811+CD3+ T cells also correlated with an increase in total cell numbers, with 80×10⁴ 2811+CD3+ present on day 15. On day 20 the percentage of 2811+CD3+ reduced to 16.45% and 17.56% when cultured with CD3/CD8, IL-7/IL-21/IL-15 this is down from 31.53% on day 15. The decrease in the percentage of 2811+CD3+ T cells also correlated with a decrease in the total number of 2811+ T cells.

Further and more detailed phenotypic analysis was performed on T cells from 2 donors to identify Tscm cells in T cells cultured in the presence of CD3/CD28 alone or in combination with IL-7, IL-15 and IL-21. The frequency of Tscm cells in humans is low, the percentage of Tscm in four healthy donors ranged from 0.64% to 3.48%. We investigated if Tscm could be expanded in the presence of CD3/CD28 alone or in combination with IL-7, IL-15 and IL-21 (FIG. 23). The largest expansion of Tscm cells was observed when T cells were cultured in the presence of anti CD3/CD28 in the presence of IL-7/IL-21 (3.51% and 6.32% Tscm) or with IL-7/IL-15/IL-21 (3.62% Tscm), for one donor this was a 5 fold expansion in Tscm cells and an 8 fold expansion for the second donor. On day 20 the percentage of Tscm cells expanded further when T cells were cultured in the presence of anti CD3/CD28 in the presence of IL-7/IL-21 (14.84% and 11.33% Tscm) or with IL-7/IL-15/IL-21 (13.67%), for one donor this was a 3 fold expansion in Tscm cells and a 9 fold expansion for the second donor (compared to day 20 media control). We next determined what percentage of Tscm cells were also positive for 2811 (FIG. 23ii). On day 0 the percentage of Tscm cells that were 2811+ was 46.89% and 63.60%. On day 15 the percentage of Tscm 2811+ cells remained similar between all the conditions, these ranged from 31.51 to 53.52%. On day 20 the percentage of Tscm 2811+ cells reduced for the majority of conditions, only media alone or T cells cultured in the presence of CD3/CD28 alone maintained a similar percentage to the day 15 results. We next determined what percentage of Tscm cells were also positive for CD3 and 2811 (FIG. 23iii). On day 15 the percentage of Tscm cells did not increase in the presence of CD3/CD28 or in combination of IL-7, IL-15, IL-21 when compared to the media only controls or the day 0 result. On day 20 the percentage of Tscm cells did not increase in the presence of CD3/CD28 or in combination of IL-7, IL-15, IL-21 when compared to the media only controls or the day 0 result.

These results show that stimulation with CD3/CD28 induced the ex vivo expansion of 2811+ cells isolated form healthy human donors. The stimulation of T cells with anti CD3/CD28 increased the frequency of 2811+ cells, this expansion was increased further when IL-7, IL-15 and IL-21 where all added to the culture, this expansion peaked 15 days after stimulation. The stimulation of T cells with anti CD3/CD28 in combination expanded the Tscm population, this expansion resulted in a 3 to 9-fold expansion of these cells.

### Example 19. T Cell Stimulated with Soluble FG2811mG1 Stimulate CD4 and CD8 T Cell Proliferation Via Fc Cross Linking

We next investigated if soluble FG2811mG1 could stimulate CD4 and CD8 T cells when cultured in the presence or absence of splenocytes. The addition of splenocytes should allow Fc crosslinking and stimulate a T cell response. Splenocytes were isolated from HHDII and HHDII/DP4 mice, pan T cells enriched (CD3+) from the HHDII splenocytes, both the HHDII T cells and HHDII/DP4 splenocytes were labelled with CFSE. HHDII T cells were then cultured with or without HHDII/DP4 splenocytes in addition to FG2811mG1, LPS or media alone. On day 15 the CD4 and CD8 proliferative responses were determined. In the absence of co culture with splenocytes only 0.14% CD4 T proliferated (CFSE^{low}) in the presence of soluble FG2811mG1, however, this was not above the media only control (0.16% CFSE^{low}) and therefore just background levels. In the absence of co culture with splenocytes only 0.02% CD8 T proliferated (CFSE^{low}) in the presence of soluble FG2811mG1, this was very similar to the media only control (0% CFSE^{low}) and therefore just background levels. Both the CD4 and CD8 T cells showed a good proliferative response to LPS (3.34%, 39.56% respectively). In the presence of co culture with splenocytes 15.2% CD4 T proliferated (CFSE^{low}) in the presence of soluble FG2811mG1 and 2.33% CD8 T proliferated (CFSE^{low}) in the presence of soluble FG2811mG1, Both the CD4 and CD8 T cells showed a good proliferative response to LPS which was enhanced in the presence of PBMCs (59.88%, 54.10% respectively).

These results show that soluble FG2811mG1 can stimulate CD4 and a CD8 proliferative response when cocultured in the presence of splenocytes (FIG. 24). Without the addition of splenocytes in the culture the T cells failed to proliferate, this shows that Fc cross linking is the mode of action of the 2811 mAb. The expansion of T cells when cocultured with splenocytes and FG2811mG1 was greater in the CD4 T cell population when compared to the CD8 T cells (15.2% vs 2.33%). These results demonstrate the potential of 2811 mAb to expand T cells ex vivo and its mode of action is via Fc cross linking.

### Embodiments

Further embodiments of the invention are described below:

1. An isolated specific binding member capable of binding to SSEA-4 (Neu5Ac(α2-3)Gal(β1-3)GalNAc(β1-3)Gal(α1-4)Gal(β1-4)Glc).

2. The binding member of embodiment 1 wherein the binding member is capable of binding SSEA-4 on glycolipids.

3. The binding member of any preceding embodiment wherein the binding member is capable of targeting stem memory T-cells (T_{SCM}).

4. The binding member of any preceding embodiment wherein the binding member is capable of inducing proliferation of stem memory T-cells (T_{SCM}).

5. The binding member according to any preceding embodiment, wherein the binding member does not bind to SSEA-3.

6. The binding member according to any preceding embodiment, wherein the binding member is mAb FG2811.72 or Chimeric FG2811.72 (CH2811/CH2811.72), or a fragment thereof.

7. The binding member according to any preceding embodiment, wherein the binding member is bispecific.

8. The binding member according to embodiment 7, wherein the bispecific binding member is additionally specific for CD3.

9. The binding member according to any preceding embodiment, wherein the binding member comprises one or more binding domains selected from the amino acid sequence of residues 27 to 38 (CDRH1), 56-65 (CDRH2) and 105-113 (CDRH3) of FIG. 2a.

10. The binding member according to any preceding embodiment, wherein the binding member comprises one or more binding domains selected from the amino acid sequence of residues 27 to 38 (CDRL1), 56-65 (CDRL2) and 105-113 (CDRL3) of FIG. 2b.

11. The binding member according to any preceding embodiment, wherein the binding member comprises a light chain variable sequence comprising one or more of LCDR1, LCDR2 and LCDR3, wherein
LCDR1 comprises SSVNY,
LCDR2 comprises DTS, and
LCDR3 comprises FQASGYPLT; and

a heavy chain variable sequence comprising one or more of HCDR1, HCDR2 and HCDR3, wherein
HCDR1 comprises GFSLNSYG,
HCDR2 comprises IWGDGST, and
HCDR3 comprises TKPGSGYAF.

12. The binding member according to any preceding embodiment, wherein the binding domain(s) are carried by a human antibody framework.

13. The binding member according to any preceding embodiment, wherein the binding member comprises a VH domain comprising residues 1 to 126 of the amino acid sequence of FIG. 2a, and/or a VL domain comprising residues 1 to 123 of the amino acid sequence of FIG. 2b.

14. The binding member according to any preceding embodiment, wherein the binding member comprises a human antibody constant region.

15. The binding member according to any preceding embodiment, wherein the binding member is an antibody, an antibody fragment, Fab, (Fab′)2, scFv, Fv, dAb, Fd or a diabody.

16. The binding member according to any preceding embodiment, wherein the binding member is an scFv comprising, in the following order, 1) leader sequence, 2) heavy chain variable region, 3) 3×GGGGS spacer, 4) light chain variable region, and 5) poly-Ala and a 6×His tag for purification.

17. The binding member according to any of embodiments 1 to 15, wherein the binding member is an scFv comprising, in the following order, 1) leader sequence, 2) light chain variable region, 3) 3×GGGGS spacer, and 4) heavy chain variable region, optionally further comprising either 5′ or 3′ purification tags.

18. The binding member according to any preceding embodiment, wherein the binding member is provided in the form of a chimeric antigen receptor (CAR).

19. The binding member according to embodiment 18, wherein the binding member is an scFv provided in the form of a chimeric antigen receptor (CAR) either in the heavy chain-light chain orientation or the light chain-heavy chain orientation.

20. The binding member according to any of embodiments 1 to 17, wherein the binding member is provided in the form of an agonist (IgG2) monoclonal antibody.

21. The binding member according to any of embodiments 1 to 17, wherein the binding member is provided in the form of an antagonist monoclonal antibody.

22. The binding member according to any preceding embodiment, wherein the binding member is monoclonal, such as a monoclonal antibody.

23. The binding member according to any preceding embodiment, wherein the binding member is a human, humanized, chimeric or veneered antibody.

24. An isolated specific binding member capable of binding specifically to SSEA-4 (Neu5Ac(α2-3)Gal(β1-3)GalNAc(β1-3)Gal(α1-4)Gal(β1-4)Glc), which competes with an isolated specific binding member as embodimented in any one of embodiments 1 to 23.

25. A binding member according to any preceding embodiment for use in therapy.

26. A binding member according to any of embodiments 1 to 24 for use in a method of the preventing, treating or diagnosing cancer.

27. A binding member according to any of embodiments 1 to 24, for use in a method of treating chronically virally infected patients.

28. A binding member according to any of embodiments 1 to 24, for use in a method of treating an autoimmune disease, HIV, adult T-cell leukaemia or graft versus host disease.

29. A method of treating or preventing cancer, comprising administering a binding member according to any of embodiments 1 to 24 to a subject in need of thereof.

30. A method of treating or preventing chronically virally infected patients, comprising administering a binding member according to any of embodiments 1 to 24 to a subject in need of thereof.

31. A method of treating or preventing an autoimmune disease, HIV, adult T-cell leukaemia or graft versus host disease, comprising administering a binding member according to any of embodiments 1 to 24 to a subject in need of thereof.

32. A method of enhancing a protective immune response against cancer comprising administering a binding member according to any of embodiments 1 to 24 to a subject in need of thereof.

33. The method of embodiment 32, wherein the binding member is prepared to be administered with a further immunogenic agent, optionally wherein the immunogenic agent is a cancer vaccine.

34. The method of embodiment 33, wherein the binding member and the further immunogenic agent are prepared to be administered simultaneously or sequentially.

35. The binding member for use of embodiments 25 or 26, or the method of embodiment 29, wherein the cancer is pancreatic, gastric, colorectal, ovarian or lung cancer.

36. The binding member for use of embodiments 25, 26 or 35, or the method of embodiment 28 or embodiment 31, wherein the binding member is administered, or prepared to be administered, alone or in combination with other treatments.

37. A nucleic acid comprising a sequence encoding a binding member according to any of embodiments 1-24.

38. The nucleic acid according to embodiment 37, wherein the nucleic acid is a construct in the form of a plasmid, vector, transcription or expression cassette.

39. A recombinant host cell which comprises the nucleic acid according to embodiment 37 or embodiment 38.

40. A method for diagnosis of cancer comprising using a binding member as embodimented in any of embodiments 1 to 24 to detect the glycans SSEA-4 (Neu5Ac(α2-3)Gal(β1-3)GalNAc(β1-3)Gal(α1-4)Gal(β1-4)Glc) attached to a glycolipid in a sample from an individual.

41. The method according to embodiment 40, wherein the pattern of glycans detected by the binding member is used to stratify therapy options for the individual.

42. A pharmaceutical composition comprising the binding member according to any of embodiments 1 to 24, and a pharmaceutically acceptable carrier.

43. The pharmaceutical composition according to embodiment 42, further comprising at least one or other pharmaceutical active.

44. The pharmaceutical composition according to embodiment 42 or embodiment 43, for use in the treatment of cancer.

45. The pharmaceutical composition according to embodiment 42 or embodiment 43, for use in the treatment of chronically virally infected patients.

46. The pharmaceutical composition according to embodiment 42 or embodiment 43, for use in the treatment of autoimmune disease, HIV, adult T-cell leukaemia or graft versus host disease.

47. A method of inducing proliferation of stem memory T-cells (T_{SCM}) ex vivo comprising contacting the stem memory T-cells (T_{SCM}) with a binding member according to any of embodiments 1 to 24.

48. A cell culture medium for inducing proliferation of stem memory T-cells (T_{SCM}) comprising a binding member according to any of embodiments 1 to 24.

49. A method of inducing proliferation of stem memory T-cells (T_{SCM}) in vivo comprising administering a subject with a binding member according to any of embodiments 1 to 24.

50. A method of identifying stem memory T-cells (T_{SCM}) by detecting the presence of SSEA-4 Neu5Ac(α2-3)Gal(β1-3)GalNAc(β1-3)Gal(α1-4)Gal(β1-4)Glc on the cell with a binding member according to any of embodiments 1 to 24.

51. A method of purifying stem memory T-cells (T_{SCM}) by detecting the presence of SSEA-4 Neu5Ac(α2-3)Gal(β1-3)GalNAc(β1-3)Gal(α1-4)Gal(β1-4)Glc on the cell with a binding member according to any of embodiments 1 to 24.

52. The method of embodiment 50 or 51 wherein the identifying or purifying is conducted in vivo or ex vivo.

53. The method of embodiment 51 or 52 wherein the binding member is used to label the stem memory T-cells (T_{SCM}) for purification.

54. A binding member substantially as described herein, optionally with reference to the accompanying figures.

### REFERENCES

Akinleye, A., P. Avvaru, M. Furqan, Y. Song, and D. Liu. 2013. ‘Phosphatidylinositol 3-kinase (PI3K) inhibitors as cancer therapeutics’, J Hematol Oncol, 6: 88.
Akinleye, A., Y. Chen, N. Mukhi, Y. Song, and D. Liu. 2013. ‘Ibrutinib and novel BTK inhibitors in clinical development’, J Hematol Oncol, 6: 59.
Altschul, S. F., W. Gish, W. Miller, E. W. Myers, and D. J. Lipman. 1990. ‘Basic local alignment search tool’, J Mol Biol, 215: 403-10.
Altschul, S. F., T. L. Madden, A. A. Schaffer, J. Zhang, Z. Zhang, W. Miller, and D. J. Lipman. 1997. ‘Gapped BLAST and PSI-BLAST: a new generation of protein database search programs’, Nucleic Acids Res, 25: 3389-402.
Ausubel, F M. 1992. Short protocols in molecular biology (John Wiley & Sons).
Barbas, C. F., 3rd, D. Hu, N. Dunlop, L. Sawyer, D. Cababa, R. M. Hendry, P. L. Nara, and D. R. Burton. 1994. ‘In vitro evolution of a neutralizing human antibody to human immunodeficiency virus type 1 to enhance affinity and broaden strain cross-reactivity’, Proc Natl Acad Sci USA, 91:3809-13.
Beers, S. A., M. J. Glennie, and A. L. White. 2016. ‘Influence of immunoglobulin isotype on therapeutic antibody function’, Blood, 127: 1097-101.
Bird, R. E., K. D. Hardman, J. W. Jacobson, S. Johnson, B. M. Kaufman, S. M. Lee, T. Lee, S. H. Pope, G. S. Riordan, and M. Whitlow. 1988. ‘Single-chain antigen-binding proteins’, Science, 242: 423-6.
Bodanzsky, M., and A. Bodanzsky. 1984. The practice of peptide synthesis (Springer Verlag: New York).
Breton, C. S., A. Nahimana, D. Aubry, J. Macoin, P. Moretti, M. Bertschinger, S. Hou, M. A. Duchosal, and J. Back. 2014. ‘A novel anti-CD19 monoclonal antibody (GBR 401) with high killing activity against B cell malignancies’, J Hematol Oncol, 7: 33.
Cahan, L. D., R. F. Irie, R. Singh, A. Cassidenti, and J. C. Paulson. 1982. ‘Identification of a human neuroectodermal tumor antigen (OFA-I-2) as ganglioside GD2’, Proc Natl Acad Sci USA, 79: 7629-33.
Chahroudi, A., G. Silvestri, and M. Lichterfeld. 2015. ‘T memory stem cells and HIV: a long-term relationship’, Curr HIV/AIDS Rep, 12: 33-40.
Chang, W. W., C. H. Lee, P. Lee, J. Lin, C. W. Hsu, J. T. Hung, J. J. Lin, J. C. Yu, L. E. Shao, J. Yu, C. H. Wong, and A. L. Yu. 2008. ‘Expression of Globo H and SSEA3 in breast cancer stem cells and the involvement of fucosyl transferases 1 and 2 in Globo H synthesis’, Proc Natl Acad Sci USA, 105: 11667-72.
Christiansen, M. N., J. Chik, L. Lee, M. Anugraham, J. L. Abrahams, and N. H. Packer. 2014. ‘Cell surface protein glycosylation in cancer’, Proteomics, 14: 525-46.
Cid, E., M. Yamamoto, M. Buschbeck, and F. Yamamoto. 2013. ‘Murine cell glycolipids customization by modular expression of glycosyltransferases’, PLoS One, 8: e64728.
Cieri, N., B. Camisa, F. Cocchiarella, M. Forcato, G. Oliveira, E. Provasi, A. Bondanza, C. Bordignon, J. Peccatori, F. Ciceri, M. T. Lupo-Stanghellini, F. Mavilio, A. Mondino, S. Bicciato, A. Recchia, and C. Bonini. 2013. ‘IL-7 and IL-15 instruct the generation of human memory stem T cells from naive precursors’, Blood, 121: 573-84.
Cooling, L., and D. Hwang. 2005. ‘Monoclonal antibody B2, a marker of neuroendocrine sympathoadrenal precursors, recognizes the Luke (LKE) antigen’, Transfusion, 45: 709-16.
Coulie, P. G., B. J. Van den Eynde, P. van der Bruggen, and T. Boon. 2014. ‘Tumour antigens recognized by T lymphocytes: at the core of cancer immunotherapy’, Nat Rev Cancer, 14: 135-46.
Dalziel, M., M. Crispin, C. N. Scanlan, N. Zitzmann, and R. A. Dwek. 2014. ‘Emerging principles for the therapeutic exploitation of glycosylation’, Science, 343: 1235681.
Daniotti, J. L., A. A. Vilcaes, V. Torres Demichelis, F. M. Ruggiero, and M. Rodriguez-Walker. 2013. ‘Glycosylation of glycolipids in cancer: basis for development of novel therapeutic approaches’, Front Oncol, 3: 306.
Darlak, K. A., Y. Wang, J. M. Li, W. A. Harris, C. R. Giver, C. Huang, and E. K. Waller. 2014. ‘Host bone marrow-derived IL-12 enhances donor T cell engraftment in a mouse model of bone marrow transplantation’, J Hematol Oncol, 7: 16.
Di Benedetto, S., E. Derhovanessian, E. Steinhagen-Thiessen, D. Goldeck, L. Muller, and G. Pawelec. 2015. ‘Impact of age, sex and CMV-infection on peripheral T cell phenotypes: results from the Berlin BASE-II Study’, Biogerontology, 16: 631-43.
Eppstein, D. A., Y. V. Marsh, M. van der Pas, P. L. Feigner, and A. B. Schreiber. 1985. ‘Biological activity of liposome-encapsulated murine interferon gamma is mediated by a cell membrane receptor’, Proc Natl Acad Sci USA, 82: 3688-92.
Fuertes Marraco, S. A., C. Soneson, L. Cagnon, P. O. Gannon, M. Allard, S. Abed Maillard, N. Montandon, N. Rufer, S. Waldvogel, M. Delorenzi, and D. E. Speiser. 2015. ‘Long-lasting stem cell-like memory CD8⁺ T cells with a naive-like profile upon yellow fever vaccination’, Sci Transl Med, 7: 282ra48.
Fuster, M. M., and J. D. Esko. 2005. ‘The sweet and sour of cancer: glycans as novel therapeutic targets’, Nat Rev Cancer, 5: 526-42.
Gang, E. J., D. Bosnakovski, C. A. Figueiredo, J. W. Visser, and R. C. Perlingeiro. 2007. ‘SSEA-4 identifies mesenchymal stem cells from bone marrow’, Blood, 109: 1743-51.
Garrido, F., T. Cabrera, A. Concha, S. Glew, F. Ruiz-Cabello, and P. L. Stern. 1993. ‘Natural history of HLA expression during tumour development’, Immunol Today, 14: 491-9.
Gattinoni, L., E. Lugli, Y. Ji, Z. Pos, C. M. Paulos, M. F. Quigley, J. R. Almeida, E. Gostick, Z. Yu, C. Carpenito, E. Wang, D. C. Douek, D. A. Price, C. H. June, F. M. Marincola, M. Roederer, and N. P. Restifo. 2011. ‘A human memory T cell subset with stem cell-like properties’, Nat Med, 17: 1290-7.
Gattinoni, L., and N. P. Restifo. 2013. ‘Moving T memory stem cells to the clinic’, Blood, 121: 567-8. Gattinoni, L., D. E. Speiser, M. Lichterfeld, and C. Bonini. 2017. ‘T memory stem cells in health and disease’, Nat Med, 23: 18-27.
Gottschling, S., K. Jensen, A. Warth, F. J. Herth, M. Thomas, P. A. Schnabel, and E. Herpel. 2013. ‘Stage-specific embryonic antigen-4 is expressed in basaloid lung cancer and associated with poor prognosis’, Eur Respir J, 41: 656-63.
Gram, H., L. A. Marconi, C. F. Barbas, 3rd, T. A. Collet, R. A. Lerner, and A. S. Kang. 1992. ‘In vitro selection and affinity maturation of antibodies from a naive combinatorial immunoglobulin library’, Proc Natl Acad Sci USA, 89: 3576-80.
Hakomori, S. 2002. ‘Glycosylation defining cancer malignancy: new wine in an old bottle’, Proc Natl Acad Sci USA, 99: 10231-3.
Hakomori, S. I. 2008. ‘Structure and function of glycosphingolipids and sphingolipids: recollections and future trends’, Biochim Biophys Acta, 1780: 325-46.
Hakomori, S., and Y. Zhang. 1997. ‘Glycosphingolipid antigens and cancer therapy, Chem Biol, 4: 97-104.
Han, E. Q., X. L. Li, C. R. Wang, T. F. Li, and S. Y. Han. 2013. ‘Chimeric antigen receptor-engineered T cells for cancer immunotherapy: progress and challenges’, J Hematol Oncol, 6: 47.
Harichandan, A., K. Sivasubramaniyan, and H. J. Buhring. 2013. ‘Prospective isolation and characterization of human bone marrow-derived MSCs’, Adv Biochem Eng Biotechnol, 129: 1-17.
Henderson, J. K., J. S. Draper, H. S. Baillie, S. Fishel, J. A. Thomson, H. Moore, and P. W. Andrews. 2002. ‘Preimplantation human embryos and embryonic stem cells show comparable expression of stage-specific embryonic antigens’, Stem Cells, 20: 329-37.
Holliger, P., T. Prospero, and G. Winter. 1993. ‘“Diabodies”: small bivalent and bispecific antibody fragments’, Proc Natl Acad Sci USA, 90: 6444-8.
Holliger, P., and G. Winter. 1993. ‘Engineering bispecific antibodies’, Curr Opin Biotechnol, 4: 446-9. Hosokawa, T., T. Kimura, S. Nada, T. Okuno, D. Ito, S. Kang, S. Nojima, K. Yamashita, T. Nakatani, Y. Hayama, Y. Kato, Y. Kinehara, M. Nishide, N. Mikami, S. Koyama, H. Takamatsu, D. Okuzaki, N. Ohkura, S. Sakaguchi, M. Okada, and A. Kumanogoh. 2017. ‘Lamtor1 Is Critically Required for CD4(+) T Cell Proliferation and Regulatory T Cell Suppressive Function’, J Immunol, 199: 2008-19.
Huang, Y. L., J. T. Hung, S. K. Cheung, H. Y. Lee, K. C. Chu, S. T. Li, Y. C. Lin, C. T. Ren, T. J. Cheng, T. L. Hsu, A. L. Yu, C. Y. Wu, and C. H. Wong. 2013. ‘Carbohydrate-based vaccines with a glycolipid adjuvant for breast cancer’, Proc Natl Acad Sci USA, 110: 2517-22.
Huston, J. S., D. Levinson, M. Mudgett-Hunter, M. S. Tai, J. Novotny, M. N. Margolies, R. J. Ridge, R. E. Bruccoleri, E. Haber, R. Crea, and et al. 1988. ‘Protein engineering of antibody binding sites: recovery of specific activity in an anti-digoxin single-chain Fv analogue produced in Escherichia coli’, Proc Natl Acad Sci USA, 85: 5879-83.
Hwang, K. J., K. F. Luk, and P. L. Beaumier. 1980. ‘Hepatic uptake and degradation of unilamellar sphingomyelin/cholesterol liposomes: a kinetic study’, Proc Natl Acad Sci USA, 77: 4030-4.
Jespers, L. S., A. Roberts, S. M. Mahler, G. Winter, and H. R. Hoogenboom. 1994. ‘Guiding the selection of human antibodies from phage display repertoires to a single epitope of an antigen’, Biotechnology (N Y), 12: 899-903.
Junghans, R. P., T. A. Waldmann, N. F. Landolfi, N. M. Avdalovic, W. P. Schneider, and C. Queen. 1990. ‘Anti-Tac-H, a humanized antibody to the interleukin 2 receptor with new features for immunotherapy in malignant and immune disorders’, Cancer Res, 50: 1495-502.
Kannagi, R., N. A. Cochran, F. Ishigami, S. Hakomori, P. W. Andrews, B. B. Knowles, and D. Solter. 1983. ‘Stage-specific embryonic antigens (SSEA-3 and -4) are epitopes of a unique globo-series ganglioside isolated from human teratocarcinoma cells’, EMBO J, 2: 2355-61.
Karlin, S., and S. F. Altschul. 1990. ‘Methods for assessing the statistical significance of molecular sequence features by using general scoring schemes’, Proc Natl Acad Sci USA, 87: 2264-8. 1993. ‘Applications and statistics for multiple high-scoring segments in molecular sequences’, Proc Natl Acad Sci USA, 90: 5873-7.
Klebanoff, C. A., L. Gattinoni, and N. P. Restifo. 2012. ‘Sorting through subsets: which T-cell populations mediate highly effective adoptive immunotherapy?’, J Immunother, 35: 651-60.
Lau, K. S., and J. W. Dennis. 2008. ‘N-Glycans in cancer progression’, Glycobiology, 18: 750-60.
Lefranc, M. P., F. Ehrenmann, S. Kossida, V. Giudicelli, and P. Duroux. 2018. ‘Use of IMGT(®) Databases and Tools for Antibody Engineering and Humanization’, Methods Mol Biol, 1827: 35-69.
Li, G., Q. Yang, Y. Zhu, H. R. Wang, X. Chen, X. Zhang, and B. Lu. 2013. ‘T-Bet and Eomes Regulate the Balance between the Effector/Central Memory T Cells versus Memory Stem Like T Cells’, PLoS One, 8: e67401.
Lou, Y. W., P. Y. Wang, S. C. Yeh, P. K. Chuang, S. T. Li, C. Y. Wu, K. H. Khoo, M. Hsiao, T. L. Hsu, and C. H. Wong. 2014. ‘Stage-specific embryonic antigen-4 as a potential therapeutic target in glioblastoma multiforme and other cancers’, Proc Natl Acad Sci USA, 111: 2482-7.
Lugli, E., M. H. Dominguez, L. Gattinoni, P. K. Chattopadhyay, D. L. Bolton, K. Song, N. R. Klatt, J. M. Brenchley, M. Vaccari, E. Gostick, D. A. Price, T. A. Waldmann, N. P. Restifo, G. Franchini, and M. Roederer. 2013. ‘Superior T memory stem cell persistence supports long-lived T cell memory’, J Clin Invest, 123: 594-9.
Lugli, E., L. Gattinoni, A. Roberto, D. Mavilio, D. A. Price, N. P. Restifo, and M. Roederer. 2013. ‘Identification, isolation and in vitro expansion of human and nonhuman primate T stem cell memory cells’, Nat Protoc, 8: 33-42.
Mahnke, Y. D., T. M. Brodie, F. Sallusto, M. Roederer, and E. Lugli. 2013. ‘The who's who of T-cell differentiation: human memory T-cell subsets’, Eur J Immunol, 43: 2797-809.
Marks, J. D., A. D. Griffiths, M. Malmqvist, T. P. Clackson, J. M. Bye, and G. Winter. 1992. ‘By-passing immunization: building high affinity human antibodies by chain shuffling’, Biotechnology (N Y), 10: 779-83.
Martin, P. J. 2014. ‘Reversing CD8+ T-cell exhaustion with DLI’, Blood, 123: 1289-90.
Mateus, J., P. Lasso, P. Pavia, F. Rosas, N. Roa, C. A. Valencia-Hernandez, J. M. Gonzalez, C. J. Puerta, and A. Cuellar. 2015. ‘Low frequency of circulating CD8⁺ T stem cell memory cells in chronic chagasic patients with severe forms of the disease’, PLoS Negl Trop Dis, 9: e3432.
Meezan, E., H. C. Wu, P. H. Black, and P. W. Robbins. 1969. ‘Comparative studies on the carbohydrate-containing membrane components of normal and virus-transformed mouse fibroblasts. II. Separation of glycoproteins and glycopeptides by sephadex chromatography’, Biochemistry, 8: 2518-24.
Metheringham, R. L., V. A. Pudney, B. Gunn, M. Towey, I. Spendlove, and L. G. Durrant. 2009. ‘Antibodies designed as effective cancer vaccines’, MAbs, 1: 71-85.
Mezzanzanica, D., S. Canevari, A. Mazzoni, M. Figini, M. I. Colnaghi, T. Waks, D. G. Schindler, and Z. Eshhar. 1998. ‘Transfer of chimeric receptor gene made of variable regions of tumor-specific antibody confers anticarbohydrate specificity on T cells’, Cancer Gene Ther, 5: 401-7.
Myers, E. W., and W. Miller. 1989. ‘Approximate matching of regular expressions’, Bull Math Biol, 51: 5-37.
Nilsson, O., F. T. Brezicka, J. Holmgren, S. Sorenson, L. Svennerholm, F. Yngvason, and L. Lindholm. 1986. ‘Detection of a ganglioside antigen associated with small cell lung carcinomas using monoclonal antibodies directed against fucosyl-GM1’, Cancer Res, 46: 1403-7.
Noto, Z., T. Yoshida, M. Okabe, C. Koike, M. Fathy, H. Tsuno, K. Tomihara, N. Arai, M. Noguchi, and T. Nikaido. 2013. ‘CD44 and SSEA-4 positive cells in an oral cancer cell line HSC-4 possess cancer stem-like cell characteristics’, Oral Oncol, 49: 787-95.
Novero, A., P. M. Ravella, Y. Chen, G. Dous, and D. Liu. 2014. ‘Ibrutinib for B cell malignancies’, Exp Hematol Oncol, 3: 4.
Nudelman, E., S. Hakomori, R. Kannagi, S. Levery, M. Y. Yeh, K. E. Hellstrom, and I. Hellstrom. 1982. ‘Characterization of a human melanoma-associated ganglioside antigen defined by a monoclonal antibody, 4.2’, J Biol Chem, 257: 12752-6.
Pearson, W. R., and D. J. Lipman. 1988. ‘Improved tools for biological sequence comparison’, Proc Natl Acad Sci USA, 85: 2444-8.
Pilipow, K., E. Scamardella, S. Puccio, S. Gautam, F. De Paoli, E. M. Mazza, G. De Simone, S. Polletti, M. Buccilli, V. Zanon, P. Di Lucia, M. lannacone, L. Gattinoni, and E. Lugli. 2018. ‘Antioxidant metabolism regulates CD8+ T memory stem cell formation and antitumor immunity’, JCI Insight, 3.
Pinto, J. P., R. K. Kalathur, D. V. Oliveira, T. Barata, R. S. Machado, S. Machado, I. Pacheco-Leyva, I. Duarte, and M. E. Futschik. 2015. ‘StemChecker: a web-based tool to discover and explore stemness signatures in gene sets’, Nucleic Acids Res, 43: W72-7.
Pluckthun, A. 1991. ‘Antibody engineering: advances from the use of Escherichia coli expression systems’, Biotechnology (N Y), 9: 545-51.
Raphael, I., S. Nalawade, T. N. Eagar, and T. G. Forsthuber. 2015. ‘T cell subsets and their signature cytokines in autoimmune and inflammatory diseases’, Cytokine, 74: 5-17.
Reff, M. E. 1993. ‘High-level production of recombinant immunoglobulins in mammalian cells’, Curr Opin Biotechnol, 4: 573-6.
Remington, R P. 1980. Remington's pharmaceutical sciences (Mack Pub. Co.).
Restifo, N. P., and L. Gattinoni. 2013. ‘Lineage relationship of effector and memory T cells’, Curr Opin Immunol, 25: 556-63.
Saito, S., H. Aoki, A. Ito, S. Ueno, T. Wada, K. Mitsuzuka, M. Satoh, Y. Arai, and T. Miyagi. 2003. ‘Human alpha2,3-sialyltransferase (ST3Gal II) is a stage-specific embryonic antigen-4 synthase’, J Biol Chem, 278: 26474-9.
Saito, S., S. Orikasa, M. Satoh, C. Ohyama, A. Ito, and T. Takahashi. 1997. ‘Expression of globo-series gangliosides in human renal cell carcinoma’, Jpn J Cancer Res, 88: 652-9.
Sambrook, J. 1989. Molecular cloning: A laboratory manual (Cold Spring Harbor Laboratory Press).
Sandstedt, J., M. Jonsson, K. Vukusic, G. Dellgren, A. Lindahl, A. Jeppsson, and J. Asp. 2014. ‘SSEA-4⁺CD34− cells in the adult human heart show the molecular characteristics of a novel cardiomyocyte progenitor population’, Cells Tissues Organs, 199: 10³-16.
Schier, R., A. McCall, G. P. Adams, K. W. Marshall, H. Merritt, M. Yim, R. S. Crawford, L. M. Weiner, C. Marks, and J. D. Marks. 1996. ‘Isolation of picomolar affinity anti-c-erbB-2 single-chain Fv by molecular evolution of the complementarity determining regions in the center of the antibody binding site’, J Mol Biol, 263: 551-67.
Schmueck-Henneresse, M., R. Sharaf, K. Vogt, B. J. Weist, S. Landwehr-Kenzel, H. Fuehrer, A. Jurisch, N. Babel, C. M. Rooney, P. Reinke, and H. D. Volk. 2015. ‘Peripheral blood-derived virus-specific memory stem T cells mature to functional effector memory subsets with self-renewal potency’, J Immunol, 194: 5559-67.
Sell, S. 1990. ‘Cancer-associated carbohydrates identified by monoclonal antibodies’, Hum Pathol, 21: 1003-19.
Shevinsky, L. H., B. B. Knowles, I. Damjanov, and D. Solter. 1982. ‘Monoclonal antibody to murine embryos defines a stage-specific embryonic antigen expressed on mouse embryos and human teratocarcinoma cells’, Cell, 30: 697-705.
Sidman, K. R., W. D. Steber, A. D. Schwope, and G. R. Schnaper. 1983. ‘Controlled release of macromolecules and pharmaceuticals from synthetic polypeptides based on glutamic acid’, Biopolymers, 22: 547-56.
Stemmer, W. P. 1994. ‘Rapid evolution of a protein in vitro by DNA shuffling’, Nature, 370: 389-91.
Stewart, J M., and J D. Young. 1984. Solid phase peptide synthesis (Pierce Chemical Company: Rockford, Ill.).
Suresh, T., L. X. Lee, J. Joshi, and S. K. Barta. 2014. ‘New antibody approaches to lymphoma therapy’, J Hematol Oncol, 7: 58.
Suzuki, Y., N. Haraguchi, H. Takahashi, M. Uemura, J. Nishimura, T. Hata, I. Takemasa, T. Mizushima, H. Ishii, Y. Doki, M. Mori, and H. Yamamoto. 2013. ‘SSEA-3 as a novel amplifying cancer cell surface marker in colorectal cancers’, Int J Oncol, 42: 161-7.
Takeshita, M., K. Suzuki, Y. Kassai, M. Takiguchi, Y. Nakayama, Y. Otomo, R. Morita, T. Miyazaki, A. Yoshimura, and T. Takeuchi. 2015. ‘Polarization diversity of human CD4+ stem cell memory T cells’, Clin Immunol, 159: 107-17.
Taylor-Papadimitriou, J., and A. A. Epenetos. 1994. ‘Exploiting altered glycosylation patterns in cancer: progress and challenges in diagnosis and therapy’, Trends Biotechnol, 12: 227-33.
Tondeur, S., S. Assou, L. Nadal, S. Hamamah, and J. De Vos. 2008. ‘[Biology and potential of human embryonic stem cells]’, Ann Biol Clin (Paris), 66: 241-7.
Torelli, A., and C. A. Robotti. 1994. ‘ADVANCE and ADAM: two algorithms for the analysis of global similarity between homologous informational sequences’, Comput Appl Biosci, 10: 3-5.
Traunecker, A., A. Lanzavecchia, and K. Karjalainen. 1991. ‘Bispecific single chain molecules (Janusins) target cytotoxic lymphocytes on HIV infected cells’, EMBO J, 10: 3655-9.
Trill, J. J., A. R. Shatzman, and S. Ganguly. 1995. ‘Production of monoclonal antibodies in COS and CHO cells’, Curr Opin Biotechnol, 6: 553-60.
van Beek, W. P., L. A. Smets, and P. Emmelot. 1973. ‘Increased sialic acid density in surface glycoprotein of transformed and malignant cells—a general phenomenon?’, Cancer Res, 33: 2913-22.
Varki, A., R. D. Cummings, J. D. Esko, H. H. Freeze, P. Stanley, J. D. Marth, C. R. Bertozzi, G. W. Hart, and M. E. Etzler. 2009. ‘Symbol nomenclature for glycan representation’, Proteomics, 9: 5398-9.
Ward, E. S., D. Gussow, A. D. Griffiths, P. T. Jones, and G. Winter. 1989. ‘Binding activities of a repertoire of single immunoglobulin variable domains secreted from Escherichia coli’, Nature, 341: 544-6.
Wright, A. J., and P. W. Andrews. 2009. ‘Surface marker antigens in the characterization of human embryonic stem cells’, Stem Cell Res, 3: 3-11.
Ye, F., Y. Li, Y. Hu, C. Zhou, Y. Hu, and H. Chen. 2010. ‘Stage-specific embryonic antigen 4 expression in epithelial ovarian carcinoma’, Int J Gynecol Cancer, 20: 958-64.
Yvon, E., M. Del Vecchio, B. Savoldo, V. Hoyos, A. Dutour, A. Anichini, G. Dotti, and M. K. Brenner. 2009. ‘Immunotherapy of metastatic melanoma using genetically engineered GD2-specific T cells’, Clin Cancer Res, 15: 5852-60.
Zhang, Y., G. Joe, E. Hexner, J. Zhu, and S. G. Emerson. 2005. ‘Host-reactive CD8+ memory stem cells in graft-versus-host disease’, Nat Med, 11: 1299-305.

1. An isolated specific binding member capable of binding specifically to SSEA-4 (Neu5Ac(α2-3)Gal(β1-3)GalNAc(β1-3)Gal(α1-4)Gal(β1-4)Glc) and targeting stem memory T-cells (T_{SCM}).

2. The binding member of claim 1 wherein the binding member is capable of binding SSEA-4 on glycolipids.

3. The binding member of claim 1, wherein the binding member is capable of inducing proliferation of stem memory T-cells (T_{SCM}).

4. The binding member according to claim 1, wherein the binding member does not bind to SSEA-3.

5. The binding member according to claim 1, wherein the binding member is mAb FG2811.72 or Chimeric FG2811.72 (CH2811/CH2811.72), or a fragment thereof.

6. The binding member according to claim 1, wherein the binding member is bispecific.

7. The binding member according to claim 1, wherein the bispecific binding member is additionally specific for CD3.

8. The binding member according to claim 1, wherein the binding member comprises one or more binding domains selected from the amino acid sequence of residues 27 to 38 (CDRH1), 56-65 (CDRH2) and 105-113 (CDRH3) of FIG. 2a.

9. The binding member according to claim 1, wherein the binding member comprises one or more binding domains selected from the amino acid sequence of residues 27 to 38 (CDRL1), 56-65 (CDRL2) and 105-113 (CDRL3) of FIG. 2b.

10. The binding member according to claim 1, wherein the binding member comprises a light chain variable sequence comprising one or more of LCDR1, LCDR2 and LCDR3, wherein
LCDR1 comprises SSVNY,
LCDR2 comprises DTS, and
LCDR3 comprises FQASGYPLT; and
a heavy chain variable sequence comprising one or more of HCDR1, HCDR2 and HCDR3, wherein
HCDR1 comprises GFSLNSYG,
HCDR2 comprises IWGDGST, and
HCDR3 comprises TKPGSGYAF.

11. The binding member according to claim 1, wherein the binding domain(s) are carried by a human antibody framework.

12. The binding member according to claim 1, wherein the binding member comprises a VH domain comprising residues 1 to 126 of the amino acid sequence of FIG. 2a, and/or a VL domain comprising residues 1 to 123 of the amino acid sequence of FIG. 2b.

13. The binding member according to claim 1, wherein the binding member is an antibody, an antibody fragment, Fab, (Fab′)2, scFv, Fv, dAb, Fd or a diabody.

14. The binding member according to claim 1, wherein the binding member is a human, humanized, chimeric or veneered antibody.

15. A binding member according to claim 1, for use in therapy.

16. A method of preventing or treating cancer in a subject in need thereof comprising administering to the subject a binding member according to any of claims claim 1.

17. A method of enhancing a protective immune response against cancer comprising administering a binding member according to claim 1 to a subject in need of thereof.

18. The method of claim 17, wherein the binding member is prepared to be administered with a further immunogenic agent, optionally wherein the immunogenic agent is a cancer vaccine.

19. A nucleic acid comprising a sequence encoding a binding member according to claim 1.

20. A method for diagnosis of cancer comprising using a binding member as claimed in claim 1 to detect the glycans SSEA-4 Neu5Ac(α2-3)Gal(β1-3)GalNAc(β1-3)Gal(α1-4)Gal(β1-4)Glc) attached to a glycolipid in a sample from an individual.

21. A pharmaceutical composition comprising the binding member according to claim 1, and a pharmaceutically acceptable carrier.

22. A method of inducing proliferation of stem memory T-cells (T_{SCM}) ex vivo comprising contacting the stem memory T-cells (T_{SCM}) with a binding member according to claim 1.

23. A cell culture medium for inducing proliferation of stem memory T-cells (T_{SCM}) comprising a binding member according to claim 1.

24. A method of identifying stem memory T-cells (T_{SCM}) by detecting the presence of S SEA-4 Neu5Ac(α2-3)Gal(β1-3)GalNAc(β1-3)Gal(α1-4)Gal(β1-4)Glc on the cell with a binding member according to claim 1.

25. A method of purifying stem memory T-cells (T_{SCM}) by detecting the presence of S SEA-4 Neu5Ac(α2-3)Gal(β1-3)GalNAc(β1-3)Gal(α1-4)Gal(β1-4)Glc on the cell with a binding member according to claim 1.


SEQUENCE LISTING (20 sequences)

SEQ ID NO: 1 - DNA, 351 bp, Artificial sequence
  Nucleotide sequence of the mature FG2811 heavy chain variable region
caggtgcagctgaaggagtcaggacctggcctggtggcgccctcacagagcctgtccatcacatgcactgtctcagggttctcattaaacagttatggtgtaagctgggttcgccagcctccaggaaagggtctggaatggctgggagtaatatggggtgacgggagcacaaattatcattcagctctcatgtccagactgcgcatcagtaaggacaactccaagagacaagttttcttaaaactgaacagtctgcaaactgatgacacagccacgtactactgtaccaaaccgggctcgggctacgcgtttgcttactggggccaagggactctggtcactgtctctagc

SEQ ID NO: 2 - protein, 117 aa, Artificial sequence
  Amino acid sequence of the mature FG2811 heavy chain variable region
QVQLKESGPGLVAPSQSLSITCTVSGFSLNSYGVSWVRQPPGKGLEWLGVIWGDGSTNYHSALMSRLRISKDNSKRQVFLKLNSLQTDDTATYYCTKPGSGYAFAYWGQGTLVTVSS

SEQ ID NO: 3 - DNA, 318 bp, Artificial sequence
  Nucleotide sequence of the mature FG2811 kappa chain variable region
gaaaatgttctcacccagtctccagcaatcatgtctgcatctccaggggaaaaagtcaccatgacctgcagtgccagttcaagtgttaattacatgcactggtaccagcagaagtcaagcacctcccccaaactctggatttatgacacatccaaactggcttctggagtcccaggtcgcttcagtggcagtgggtctggaaactcttactctctcacgatcaggaccatggaggctgaagatgttgccacttatttctgttttcaggcgagtgggtacccgctcacgttcggtggtgggaccaagctggagctgaaa

SEQ ID NO: 4 - protein, 106 aa, Artificial sequence
  Amino acid sequence of the mature FG2811 kappa chain variable region
ENVLTQSPAIMSASPGEKVTMTCSASSSVNYMHWYQQKSSTSPKLWIYDTSKLASGVPGRFSGSGSGNSYSLTIRTMEAEDVATYFCFQASGYPLTFGGGTKLELK

SEQ ID NO: 5 - DNA, 972 bp, Artificial sequence
  Nucleotide sequence of mIgG1
gctaaaacgacacccccatctgtctatccactggcccctggttctgctgcccaaactaactccatggtgaccctgggatgcctggtcaagggctatttccctgagccagtgacagtgacctggaactctggttccctgtccagcggtgtgcacaccttcccagctgtcctgcagtctgacctctacactctgagcagctcagtgactgtcccctccagcacctggcccagcgagaccgtcacctgcaacgttgcccacccggccagcagcaccaaggtggacaagaaaattgtgcccagggattgtggttgtaagccttgcatatgtacagtcccagaagtatcatctgtcttcatcttccccccaaagcccaaggatgtgctcaccattactctgactcctaaggtcacgtgtgttgtggtagacatcagcaaggatgatcccgaggtccagttcagctggtttgtagatgatgtggaggtgcacacagctcagacgcaaccccgggaggagcagttcaacagcactttccgctcagtcagtgaacttcccatcatgcaccaggactggctcaatggcaaggagttcaaatgcagggtcaacagtgcagctttccctgcccccatcgagaaaaccatctccaaaaccaaaggcagaccgaaggctccacaggtgtacaccattccacctcccaaggagcagatggccaaggataaagtcagtctgacctgcatgataacagacttcttccctgaagacattactgtggagtggcagtggaatgggcagccagcggagaactacaagaacactcagcccatcatggacacagatggctcttacttcgtctacagcaagctcaatgtgcagaagagcaactgggaggcaggaaatactttcacctgctctgtgttacatgagggcctgcacaaccaccatactgagaagagcctctcccactctcctggtaaa

SEQ ID NO: 6 - protein, 324 aa, Artificial sequence
  Amino acid sequence of mIgG1
AKTTPPSVYPLAPGSAAQTNSMVTLGCLVKGYFPEPVTVTWNSGSLSSGVHTFPAVLQSDLYTLSSSVTVPSSTWPSETVTCNVAHPASSTKVDKKIVPRDCGCKPCICTVPEVSSVFIFPPKPKDVLTITLTPKVTCVVVDISKDDPEVQFSWFVDDVEVHTAQTQPREEQFNSTFRSVSELPIMHQDWLNGKEFKCRVNSAAFPAPIEKTISKTKGRPKAPQVYTIPPPKEQMAKDKVSLTCMITDFFPEDITVEWQWNGQPAENYKNTQPIMDTDGSYFVYSKLNVQKSNWEAGNTFTCSVLHEGLHNHHTEKSLSHSPGK

SEQ ID NO: 7 - DNA, 978 bp, Artificial sequence
  Nucleotide sequence of hIgG2
gcttccaccaagggcccatcggtcttccccctggcgccctgctccaggagcacctccgagagcacagcggccctgggctgcctggtcaaggactacttccccgaaccggtgacggtgtcgtggaactcaggcgctctgaccagcggcgtgcacaccttcccggctgtcctacagtcctcaggactctactccctcagcagcgtggtgaccgtgccctccagcaacttcggcacccagacctacacctgcaacgtagatcacaagcccagcaacaccaaggtggacaagacagttgagcgcaaatgttgtgtcgagtgcccaccgtgcccagcaccacctgtggcaggaccgtcagtcttcctcttccccccaaaacccaaggacaccctcatgatctcccggacccctgaggtcacgtgcgtggtggtggacgtgagccacgaagaccccgaggtccagttcaactggtacgtggacggcgtggaggtgcataatgccaagacaaagccacgggaggagcagttcaacagcacgttccgtgtggtcagcgtcctcaccgtcgtgcaccaggactggctgaacggcaaggagtacaagtgcaaggtctccaacaaaggcctcccagcccccatcgagaaaaccatctccaaaaccaaagggcagccccgagaaccacaggtgtacaccctgcccccatcccgggaggagatgaccaagaaccaggtcagcctgacctgcctggtcaaaggcttctaccccagcgacatcgccgtggagtgggagagcaatgggcagccggagaacaactacaagaccacacctcccatgctggactccgacggctccttcttcctctacagcaagctcaccgtggacaagagcaggtggcagcaggggaacgtcttctcatgctccgtgatgcatgaggctctgcacaaccactacacgcagaagagcctctccctgtctccgggtaaa

SEQ ID NO: 8 - protein, 326 aa, Artificial sequence
  Amino acid sequence of hIgG2
ASTKGPSVFPLAPCSRSTSESTAALGCLVKDYFPEPVTVSWNSGALTSGVHTFPAVLQSSGLYSLSSVVTVPSSNFGTQTYTCNVDHKPSNTKVDKTVERKCCVECPPCPAPPVAGPSVFLFPPKPKDTLMISRTPEVTCVVVDVSHEDPEVQFNWYVDGVEVHNAKTKPREEQFNSTFRVVSVLTVVHQDWLNGKEYKCKVSNKGLPAPIEKTISKTKGQPREPQVYTLPPSREEMTKNQVSLTCLVKGFYPSDIAVEWESNGQPENNYKTTPPMLDSDGSFFLYSKLTVDKSRWQQGNVFSCSVMHEALHNHYTQKSLSLSPGK

SEQ ID NO: 9 - DNA, 990 bp, Artificial sequence
  Nucleotide sequence of hIgG1
gcttccaccaagggcccatcggtcttccccctggcaccctcctccaagagcacctctgggggcacagcggccctgggctgcctggtcaaggactacttccccgaaccggtgacggtgtcgtggaactcaggcgccctgaccagcggcgtgcacaccttcccggctgtcctacagtcctcaggactctactccctcagcagcgtggtgaccgtgccctccagcagcttgggcacccagacctacatctgcaacgtgaatcacaagcccagcaacaccaaggtggacaagaaagttgagcccaaatcttgtgacaaaactcacacatgcccaccgtgcccagcacctgaactcctggggggaccgtcagtcttcctcttccccccaaaacccaaggacaccctcatgatctcccggacccctgaggtcacatgcgtggtggtggacgtgagccacgaagaccctgaggtcaagttcaactggtacgtggacggcgtggaggtgcataatgccaagacaaagccgcgggaggagcagtacaacagcacgtaccgtgtggtcagcgtcctcaccgtcctgcaccaggactggctgaatggcaaggagtacaagtgcaaggtctccaacaaagccctcccagcccccatcgagaaaaccatctccaaagccaaagggcagccccgagaaccacaggtgtacaccctgcccccatcccgggatgagctgaccaagaaccaggtcagcctgacctgcctggtcaaaggcttctatcccagcgacatcgccgtggagtgggagagcaatgggcagccggagaacaactacaagaccacgcctcccgtgctggactccgacggctccttcttcctctacagcaagctcaccgtggacaagagcaggtggcagcaggggaacgtcttctcatgctccgtgatgcatgaggctctgcacaaccactacacgcagaagagcctctccctgtctccgggtaaa

SEQ ID NO: 10 - protein, 330 aa, Artificial sequence [sequence omitted]

SEQ ID NO: 11 - DNA, 321 bp, Artificial sequence
  Nucleotide sequence of murine Kappa constant region
cgtgcagatgctgcaccaactgtatcgatcttcccaccatccagtgagcagttaacatctggaggtgcctcagtcgtgtgcttcttgaacaacttctaccccaaagacatcaatgtcaagtggaagattgatggcagtgaacgacaaaatggcgtcctgaacagttggactgatcaggacagcaaagacagcacctacagcatgagcagcaccctcacgttgaccaaggacgagtatgaacgacataacagctatacctgtgaggccactcacaagacatctacttcacccattgtcaagagcttcaacaggaatgagtgt

SEQ ID NO: 12 - protein, 107 aa, Artificial sequence
  Amino acid sequence of murine Kappa constant region
RADAAPTVSIFPPSSEQLTSGGASVVCFLNNFYPKDINVKWKIDGSERQNGVLNSWTDQDSKDSTYSMSSTLTLTKDEYERHNSYTCEATHKTSTSPIVKSFNRNEC

SEQ ID NO: 13 - DNA, 321 bp, Artificial sequence
  Nucleotide sequence of human Kappa constant region
cgtacggtagcggccccatctgtcttcatcttcccgccatctgatgagcagttgaaatctggaactgcctctgttgtgtgcctgctgaataacttctatcccagagaggccaaagtacagtggaaggtggataacgccctccaatcgggtaactcccaggagagtgtcacagagcaggacagcaaggacagcacctacagcctcagcagcaccctgacgctgagcaaagcagactacgagaaacacaaagtctacgcctgcgaagtcacccaccagggcctgagctcgcccgtcacaaagagcttcaacaggggagagtgt

SEQ ID NO: 14 - protein, 107 aa, Artificial sequence [sequence omitted]

SEQ ID NO: 15 - protein, 5 aa, Artificial sequence
  LCDR1 amino acid sequence
SSVNY

SEQ ID NO: 16 - protein, 3 aa, Artificial sequence
  LCDR2 amino acid sequence
DTS

SEQ ID NO: 17 - protein, 9 aa, Artificial sequence
  LCDR3 amino acid sequence
FQASGYPLT

SEQ ID NO: 18 - protein, 8 aa, Artificial sequence
  HCDR1 amino acid sequence
GFSLNSYG

SEQ ID NO: 19 - protein, 7 aa, Artificial sequence
  HCDR2 amino acid sequence
IWGDGST

SEQ ID NO: 20 - protein, 9 aa, Artificial sequence
  HCDR3 amino acid sequence
TKPGSGYAF